(19) United States
(12) Patent Application Publication
(10) Pub. No.: US 2025/0382381 A1
(43) Pub. Date: Dec. 18, 2025

(54) TARGETING ART1 FOR CANCER IMMUNOTHERAPY

(71) Applicants: Cornell University, Ithaca, NY (US); Tri-Institutional Therapeutics Discovery Institute, New York, NY (US)

(72) Inventors: Brendon M. Stiles, New York, NY (US); Timothy McGraw, New York, NY (US); Ivo C. Lorenz, Brooklyn, NY (US); Thomas E. White, New York, NY (US); Abdul G. Khan, West Babylon, NY (US)

(21) Appl. No.: 18/836,705

(22) PCT Filed: Feb. 7, 2023
(86) PCT No.: PCT/US2023/062151

§ 371 Date: Aug. 7, 2024

Related U.S. Application Data
(60) Provisional application No. 63/307,502, filed on Feb. 7, 2022.

(51) Int. Cl. C07K 16/30; A61K 38/00; A61P 35/04; C12N 5/0781; C12N 5/09; C12N 5/16; C12N 15/11

(52) U.S. Cl. CPC C07K 16/30; A61K 38/00; A61P 35/04; C12N 5/0635; C12N 5/0694; C12N 5/163; C12N 15/11; C07K 2317/565; C07K 2317/567; C07K 2317/76; C12N 2510/04

(57) ABSTRACT

Antibodies that bind human ART1 and uses thereof are provided.

### CROSS-REFERENCE TO RELATED APPLICATIONS

This application claims the benefit of the filing date of U.S. application No. 63/307,502, filed on Feb. 7, 2022, the disclosure of which is incorporated by reference herein.

### STATEMENT OF GOVERNMENT RIGHTS

This invention was made with government support under grant W81XWH1910422 awarded by the Department of Defense. The government has certain rights in the invention.

### INCORPORATION BY REFERENCE BF SEQUENCE LISTING

A Sequence Listing is provided herewith as an xml file, “2306900.xml” created on Feb. 2, 2023 and having a size of 99,523 bytes. The content of the xml file is incorporated by reference herein in its entirety.

### BACKGROUND

ADP-ribosyl transferase 1 (ART1), an ARTC family mono-ADP-ribosyltransferase, functions extracellularly to ADP-ribosylate cell surface proteins or target soluble proteins in the local tumor microenvironment. Mono-ADP-ribosylation can be blocked by arginine analogues and nicotinamide mimics that act as competitive inhibitors. Such analogues include the antibiotic novobiocin, which has previously been utilized safely in lung cancer trials, based upon other non-targeted mechanisms. A second known inhibitor of mono-ADP-ribosylation is meta-iodobenzylguanidine (MIBG), a norepinephrine analogue with a long safety record of use in medical imaging procedures. MIBG may exert inhibitory effects on the metastatic properties of a hepatocellular carcinoma cell line, possibly through inhibition of mono-ADP-ribosylation. In addition to these well described inhibitors of mono-ADP-ribosylation, a tremendous effort has been undertaken by pharmaceutical companies to develop small molecule inhibitors of intracellular poly- and mono-ADP-ribosylation. Because these drugs are designed to compete with NAD⁺ at the enzyme active site and because they are largely based on benzamide or purine structures, the agents also have the potential to inhibit other enzymes that utilize NAD⁺, including ART1. However, they are not specific for ART1 mono-ribosyltransferase activity.

### SUMMARY

The disclosure provides for selective inhibitors of ART1, e.g., inhibitors of mono-ADP-ribosylation, to suppress tumor growth and facilitate cytotoxicity of immune cells towards ART1 expressing cells such as cancer cells including lung cancer cells. In particular, the disclosure provides for antibodies, fragments thereof and single chain ART1 binding polypeptides, targeting ART1, an extracellular mono-ADP ribosyltransferase, e.g., antibodies that bind ART1, for the treatment of diseases including cancer. For example, as disclosed herein, ART1 is highly expressed in multiple human non-small cell lung cancer (NSCLC) lines of distinct driver mutation status and strong ART1 protein expression was observed in over half of human lung adenocarcinomas. Experiments in a genetically engineered murine adenocarcinoma model suggest that ART1 overexpression plays an important role in survival and metastatic outgrowth of disseminated tumor cells, likely due to protection from immune cells in the tumor microenvironment. Thus, compounds that specifically inhibit ART1 or its function, such as anti-human ART1 specific antibodies or portions thereof, can be used as targeted therapeutics in ART1-overexpressing cancers, such as in NSCLC patients, to limit metastatic spread of cancer by facilitating immune-mediated destruction of disseminated cells. As an extracellular enzymatic target, ART1 is highly druggable by various therapeutic modalities including antibodies and fragments thereof. Moreover, inhibitors of ADP-ribosylation may be used in a combination therapy with cytotoxic chemotherapy or with immune checkpoint inhibitors. The ART1 inhibitors are useful in a wide-variety of cancers, including for example colon cancer or breast cancer. The inhibitors may be useful in inhibiting cancer progression and/or metastasis.

The disclosure provides an isolated antibody that binds human ART1 and optionally murine ART1. The antibody may be produced from a vertebrate cell, e.g., one transfected with nucleic acid sequences encoding an anti-ART1 antibody or antigen binding fragment thereof, or a polypeptide, that inhibits human ART1 activity, from an immune cell or a hybridoma, e.g., which expresses a monoclonal antibody. In one embodiment, an isolated monoclonal antibody that binds human and mouse ART1 is provided. The nucleic acid sequences encoding an anti-ART1 antibody or antigen binding fragment thereof, or the polypeptide, may be operably linked to a promoter, such as a heterologous promoter. The cell may be a mammalian cell, a primate cell, an insect cell or a plant cell. In one embodiment, an isolated nucleic acid comprising a promoter operably linked to a nucleotide sequence which encodes at least the variable region of a human or murine heavy or light chain that binds human and/or mouse ART1 is provided.

In one embodiment, an expression cassette is provided comprising nucleic acid sequences encoding an anti-ART1 antibody or antigen binding fragment thereof, or a polypeptide, that inhibits human ART1 activity, which sequence encodes QVTLKESGPVLVKPTETLTLTCTVSGFSLSNARMGVSWIRQPPGKALEWLAHIFSNDEKSYSTSLKSRLTISKD TSKSQVVLTMTNMDPVDTATYYCARIYGGDSWGYFDNWGQGTLVTVSS (SEQ ID NO:1); QIVLTQSPAIMSASLGEKVTMTCSASSSVSYMHWYQQKSGTSPKRWIYDTSKLASGVPARFSGSGSGTSYSL TISSMEAGDAATYYCQQWSSNPPTFGAGTKLELK (SEQ ID NO:2); QVQLQESGPGLVKPSETLSLTCTVSGGSISSYYWSWIRQPAGKGLEWIGRISTSGFTNYNPSLKSRVTMSVD SSKNQFSLKLSSLTAADTAVYYCARDGWGRVFDIWGLGTMVTVSS (SEQ ID NO:3); or EIVLTQSPGTLSLSPGERATLSCRASQSVSSSYLAWYQQKPGQAPRLLIYGASSRATGIPDRFSGSGSGTDFT LTISRLEPEDFAVYYCQQYGSSTFGPGTKVDIK (SEQ ID NO:4), or a polypeptide with at least 80%, 85%, 90%, 92%, 94%, 95%, 96%, 97%, 98%, or 99% amino acid sequence identity thereto.

In one embodiment, an expression cassette is provided comprising nucleic acid sequences encoding an anti-ART1 antibody or antigen binding fragment thereof, or a polypeptide, that inhibits human ART1 activity, which sequence encodes a plurality of CDRs having S NARMGVS (SEQ ID NO:21), HIFSNDEKSYSTSLKS (SEQID NO: 22), IYGGDSWGYFDN (SEQ ID NO:23), QVTLKESGPVLVKPTETLTLTCTVSGFSLS (SEQ ID NO:24); SSSVSY (SEQ ID NO:28), SSVSY (SEQ ID NO: 81), DTS (SEQ ID NO:29), or QQWSSNPPT (SEQ ID NO:30); or GFSLSNARMG (SEQ ID NO:66) IFSNDEK (SEQ ID NO:67), ARIYGGDSWGYFDN (SEQ ID NO:68); SSSVSY (SEQ ID NO:28), DTS (SEQ ID NO: 29), or QQWSSNPPT (SEQ ID NO:30), optionally including one or more framework regions having QVTLKESGPVLVKPTETLTLTCTVSGFSLS (SEQ ID NO:24), WIRQPPGKALEWLA (SEQ ID NO:25), RLTISKDTSKSQVVLTMTNMDPVDTATYYCAR (SEQ ID NO:26) or WGQGTLVTVSS (SEQ ID NO:27), or QIVLTQSPAIMSASLGEKVTMTCSA (SEQ ID NO:31), MHWYQQKSGTSPKRWIY (SEQ ID NO:32), KLASGVPARFSGSGSGTSYSLTISSMEAGDAATYYC (SEQ ID NO:33) orFGAGTKLELK (SEQ ID NO:34), or DIQLTQSPSFLSASVGDRVTITCRA (SEQ ID NO:51), MHWYQQKPGTSPKRLIY (SEQ ID NO:52), KLASGVPSRFSGSGSGTEYTLTISSLQPEDFATYYC (SEQ ID NO:53), or FGQGTKLEIKRTVAAPSVFIFPPSDEQLKSGTASVVCLLNNFYPREAKVQWKVDNALQSGNSQESVTEQDSK DSTYSLSSTLTLSKADYEKHKVYACEVTHQGLSSPVTKSFNRGEC (SEQ ID NO:54) or DIQMTQSPSSLSASVGDRVTITCSA (SEQ ID NO:55), MHWYQQKPGTSPKRLIY (SEQ ID NO:56), KLASGVPSRFSGSGSGTDYTLTISSLQPEDFATYYC (SEQ ID NO:57) or FGQGTKLEIKRTVAAPSVFIFPPSDEQLKSGTASVVCLLNNFYPREAKVQWKVDNALQSGNSQESVTEQDSK DSTYSLSSTLTLSKADYEKHKVYACEVTHQGLSSPVTKSFNRGEC (SEQ ID NO:58), or EIVLTQSPATLSLSPGERATLSCRA (SEQ ID NO:59), MHWYQQKPGTSPRRLIY (SEQ ID NO:60), KLATGIPARFSGSGSGTDYTLTISSLEPEDFAVYYC (SEQ ID NO:61) or TFGQGTKLEIKRTVAAPSVFIFPPSDEQLKSGTASVVCLLNNFYPREAKVQWKVDNALQSGNSQESVTEQDS KDSTYSLSSTLTLSKADYEKHKVYACEVTHQGLSSPVTKSFNRGEC (SEQ ID NO:62), or QVTLKESGPVLVKPTETLTLTCTVS (SEQ ID NO:71), VSWIRQP PGKALEWLAH (SEQ ID NO:72), SYSTSLKSRLTISKDTSKSQVVLTMTNMDPVDTATYYC (SEQ ID NO:73) or WGQGTLVTVSS (SEQ ID NO: 74), or DIQLTQSPSFLSASVGDRVTITCRAS (SEQ ID NO:76), YMHWYQQKPGTS PKRLIY (SEQ ID NO: 77), KLASGVPSRFSGSGSGTEYTLTISSLOPEDFATY YC (SEQ ID NO:78), or FGQGTKLEIK (SEQ ID NO: 79), or a polypeptide with at least 80%, 85%, 90%, 92%, 94%, 95%, 96%, 97%, 98%, or 99% amino acid sequence identity thereto.

In one embodiment, an expression cassette is provided comprising nucleic acid sequences encoding an anti-ART1 antibody or antigen binding fragment thereof, or a polypeptide, that inhibits human ART1 activity, which sequence encodes a plurality of CDRs having GGSISSYY (SEQ ID NO:35), ISTSGFT (SEQ ID NO: 36), ARDGWGRVFDI (SEQ ID NO:37) or QSVSSSY (SEQ ID NO:42), GAS (SEQ ID NO:43) or QQYGSST (SEQ ID NO:44), optionally including one or more framework regions having QVQLQESGPGLVKPSETLSLTCTVS (SEQ ID NO:38), WSWIRQPAGKGLEWIGR (SEQ ID NO:39), NYNPSLKSRVTMSVDSSKNQFSLKLSSLTAADTAVYYC (SEQ ID NO:40) or WGLGTMVTVSS (SEQ ID NO: 41), or EIVLTQSPGTLSLSPGERATLSCRAS (SEQ ID NO:45), LAWYQQKPGQAPRLLIY (SEQ ID NO: 46), SRATGIPDRFSGSGSGTDFTLTISRLEPEDFAVYYC (SEQ ID NO:47), or FGPGTKVDIK (SEQ ID NO: 63), a polypeptide with at least 80%, 85%, 90%, 92%, 94%, 95%, 96%, 97%, 98%, or 99% amino acid sequence identity thereto.

In one embodiment, the antibody or antigen binding fragment thereof, or polypeptide, comprises CDRs comprising SEQ ID NO:21, SEQ ID NO:22 and SEQ ID NO:23, and SEQ ID NO:28, SEQ ID NO:29 and SEQ ID NO:30.

In one embodiment, the antibody or antigen binding fragment thereof, or polypeptide, comprises CDRs comprising SEQ ID NO:35, SEQ ID NO:36 and SEQ ID NO:37, and QSVSSSY (SEQ ID NO:42), GAS (SEQ ID NO:43) and SEQ ID NO:44.

In one embodiment, the antibody or antigen binding fragment thereof, or polypeptide, comprises CDRs comprising SEQ ID NO:66, SEQ ID NO:67 and SEQ ID NO:68, and SEQ ID NO:81, SEQ ID NO:29 and SEQ ID NO:30.

In one embodiment, a framework region in the antibody or antigen binding fragment thereof, or polypeptide, comprises SEQ ID NO:24, SEQ ID NO:25, SEQ ID NO:26 and/or WGQGTLVTVSS (SEQ ID NO: 27), or a sequence having one, two, three, four, or five conservative amino acid substitutions, and optionally one two or three non-conservative substitutions.

In one embodiment, a framework region in the antibody or antigen binding fragment thereof, or polypeptide, comprises SEQ ID NO:31, SEQ ID NO:32, SEQ ID NO:33, and/or SEQ ID NO:34, or a sequence having one, two, three, four or five conservative amino acid substitutions, and optionally one two or three non-conservative substitutions or a polypeptide with at least 80%, 82%, 84%, 85%, 87%, 88%, 89%, 90%, 92%, 94%, 95%, 96%, 97%, 98%, or 99% amino acid sequence identity thereto.

In one embodiment, a framework region in the antibody or antigen binding fragment thereof, or polypeptide, comprises SEQ ID NO:38, SEQ ID NO:39, SEQ ID NO:40, and/or SEQ ID NO:41, or a sequence having one, two, three, four or five conservative amino acid substitutions, and optionally one two or three non-conservative substitutions or a polypeptide with at least 80%, 82%, 84%, 85%, 87%, 88%, 89%, 90%, 92%, 94%, 95%, 96%, 97%, 98%, or 99% amino acid sequence identity thereto . . . .

In one embodiment, a framework region in the antibody or antigen binding fragment thereof, or polypeptide, comprises SEQ ID NO:45, SEQ ID NO:46, SEQ ID NO:47 and/or SEQ ID NO:63, or a sequence having one, two, three, four or five conservative amino acid substitutions, and optionally one two or three non-conservative substitutions or a polypeptide with at least 80%, 82%, 84%, 85%, 87%, 88%, 89%, 90%, 92%, 94%, 95%, 96%, 97%, 98%, or 99% amino acid sequence identity thereto.

In one embodiment, the antibody or antigen binding fragment thereof, or polypeptide binds to a portion of human or mouse ART1 from residue 70 to 100, 170 to 182, 192 to 206 or 230 to 245, e.g., relative to, e.g. SEQ ID NO:90.

In one embodiment, a CDR has one, two or three amino acid substitutions relative to NARMGVS (SEQ ID NO:21), HIFSNDEKSYSTSLKS (SEQ ID NO:22) or IYGGDSWGYFDN (SEQ ID NO:23). For example, a CDR with one or two substitutions relative to NARMGVS (SEQ ID NO:21) has NAHMGVS (SEQ ID NO: 93), QARMGIS (SEQ ID NO:94) or NGRMGVS (SEQ ID NO:95); a CDR with one, two or three substitutions relative to HIFSNDEKSYSTSLKS (SEQ ID NO:22) has HIFSNDEKSYSTSIKS (SEQ ID NO:96), HLFSNDEKSYSTSIKS (SEQ ID NO:97) or HIFTNDEKSYSSSLKS (SEQ ID NO:98); and a CDR with one or a few substitutions relative to IYGGDSWGYFDN (SEQ ID NO:23) has IYGGADSWGYFEN (SEQ ID NO:99), IYGGDSWAYFDN (SEQ ID NO:100), or LYGIDSWGYFDN (SEQ ID NO:101)

In one embodiment, a CDR has one, two or three amino acid substitutions relative to GFSLSNARMG SEQ ID NO:66), IFSNDEK (SEQ ID NO:67) or ARIYGGDSWGYFDN (SEQ ID NO:68. For example, a CDR with one or two substitutions relative to_GFSLSNARMG (SEQ ID NO:66) has GFSISNARMG (SEQ ID NO:102), GFSASNTRMG (SEQ ID NO:103) or GFSISNLRMA (SEQ ID NO:104). For example, a CDR with one or two substitutions relative to IFSNDEK (SEQ ID NO:67) has LFSNDEK (SEQ ID NO: 105) or IFSNEDK (SEQ ID NO:106). For example, a CDR with one, two or three substitutions relative to ARIYGGDSWGYFDN (SEQ ID NO: 68) has GRIYGGDSWGYFDN (SEQ ID NO:107), ARIYAADSWGYFDN (SEQ ID NO:108) or IRAYGGDSWLYFDN (SEQ ID NO:109).

A composition having an ART1 expression cassette, e.g., in a gene expression vector, ART1 binding antibodies or antigen binding fragments thereof, or polypeptides that bind ART1, may be employed in in vitro and in vivo methods. For example, the composition may be employed to inhibit or treat cancer in a mammal, e.g., by administering to the mammal an effective amount of the composition. The mammal may have lung cancer, e.g., non-small cell lung cancer, colon cancer, melanoma, glioblastoma, breast cancer, or colorectal cancer. In one embodiment, the mammal is a human. In one embodiment, the amount is effective to inhibit ART1 enzymatic activity, decrease tumor burden, inhibit metastases, enhance immune-mediated anti-tumor activity, or increase survival. In one embodiment, the mammal has an ART1 overexpressing tumor. The composition may be employed to prevent or inhibit ART1-mediated immunosuppression in a mammal or to enhance an immune response in a mammal in need thereof, e.g., a mammal having an ART1 overexpressing tumor, e.g., a mammal having NSCLC, colon cancer or melanoma.

In one embodiment, an anti-ART1 antibody is provided that binds to and/or inhibits the activity of human ART1 and/or murine ART1.

Further provided is an isolated cell comprising an expression cassette comprising a heterologous promoter operably linked to nucleic acid sequences encoding an anti-human ART1 antibody, or an antigen binding fragment thereof, or a polypeptide, that inhibits human ART1 activity, wherein the antibody, the antigen binding fragment thereof, or the polypeptide has: i) a variable region comprising a first complementarity determining region (CDR) comprising GFSLSNARM (SEQ ID NO:66) or NARMGVS (SEQ ID NO: 21) operably linked to a second CDR comprising IFSNDEK (SEQ ID NO:67) or HIFSNDEKSYSTSLKS (SEQ ID NO:22) operably linked to a third CDR comprising ARIYGGDSWGYFDN (SEQ ID NO:68) or IYGGDSWGYFDN (SEQ ID NO:23); and/or ii) a variable region comprising a first CDR comprising SSVSY (SEQ ID NO:81) or SSSVSY (SEQ ID NO:28) operably linked to a second CDR comprising DTS (SEQ ID NO: 29) operably linked to a third CDR comprising QQWSSNPPT (SEQ ID NO:30); or iii) a variable region comprising a first CDR comprising GGSISSYY (SEQ ID NO:35) operably linked to a second CDR comprising ISTSGFT (SEQ ID NO:36) operably linked to a third CDR comprising ARDGWGRVFDI (SEQ ID NO:37); and/or iv) a variable region comprising a first CDR comprising QSVSSSY (SEQ ID NO:42) operably linked to a second CDR comprising GAS (SEQ ID NO:43) operably linked to a third CDR comprising QQYGSST (SEQ ID NO: 44). In one embodiment, the cell comprises or expresses a heavy Ig chain comprises a variable region comprising
QVTLKESGPVLVKPTETLTLTCTVSGFSLSNARMGVSWIRQPPGKALEWLAHIFSNDEKSYSTSLKSRLTISKD TSKSQVVLTMTNMDPVDTATYYCARIYGGDSWGYFDNWGQGTLVTVSS (SEQ ID NO:1) or QVTLKESGPVLVKPTETLTLTCTVSGFSLSNARMGVSWIRQPPGKALEWLAH IFSNDEK
SYSTSLKSRLTISKDTSKSQVVLTMTNMDPVDTATYYCARIYGGDSWGYFDNWGQGTLVTVSS (SEQ ID NO: 82); or a light Ig chain ii) comprises a variable region comprising QIVLTQSPAIMSASLGEKVTMTCSASSSVSYMHWYQQKSGTSPKRWIYDTSKLASGVPARFSGSGSGTSYSL TISSMEAGDAATYYCQQWSSNPPTFGAGTKLELK (SEQ ID NO:2) or DIQLTQSPSFLSASVGDRVTITCRASSSVSYMHWYQQKPGTS PKRLIYDTS KLASGVPSRFSGSGSGTEYTLTISSLQPEDFATYYCQQWSSNPPTFGQGTKLEIK (SEQ ID NO:83). In one embodiment, the cell has or expressed a heavy Ig chain comprising iii) comprises a variable region comprising:
QVQLQESGPGLVKPSETLSLTCTVSGGSISSYYWSWIRQPAGKGLEWIGRISTSGFTNYNPSLKSRVTMSVD SSKNQFSLKLSSLTAADTAVYYCARDGWGRVFDIWGLGTMVTVSS (SEQ ID NO:3); or a light Ig chain comprising iv) comprises a variable region comprising EIVLTQSPGTLSLSPGERATLSCRASQSVSSSYLAWYQQKPGQAPRLLIYGASSRATGIPDRFSGSGSGTDFT LTISRLEPEDFAVYYCQQYGSSTFGPGTKVDIK (SEQ ID NO:4). In one embodiment, the cell has or expresses a light Ig chain comprising ii) comprises a variable region comprising
DIQLTQSPSFLSASVGDRVTITCRASSSVSYMHWYQQKPGTSPKRLIYDTSKLASGVPSRFSGSGSGTEYTLT ISSLQPEDFATYYCQQWSSNPPTFGQGTKL (SEQ ID NO:69);
DIQMTQSPSSLSASVGDRVTITCSASSSVSYMHWYQQKPGTSPKRLIYDTSKLASGVPSRFSGSGSGTDYTL TISSLOPEDFATYYCQQWSSNPPTFGQGTKL (SEQ ID NO:84); or
EIVLTQSPATLSLSPGERATLSCRASSSVSYMHWYQQKPGTSPRRLIYDTSKLATGIPARFSGSGSGTDYTLTI SSLEPEDFAVYYCQQWSSNPPTFGQGTKL (SEQ ID NO:87). In one embodiment, the cell is a mammalian cell. In one embodiment, the cell is a primate cell. In one embodiment, the cell is a human cell.

Also provided is a hybridoma comprising nucleic acid sequences encoding an anti-human ART1 monoclonal antibody that inhibits human ART1 activity, wherein the antibody has:
i) a variable region comprising a first complementarity determining region (CDR) comprising GFSLSNARM (SEQ ID NO:66) or NARMGVS (SEQ ID NO:21) operably linked to a second CDR comprising IFSNDEK (SEQ ID NO:67) or HIFSNDEKSYSTSLKS (SEQ ID NO:22) operably linked to a third CDR comprising ARIYGGDSWGYFDN (SEQ ID NO:68) or IYGGDSWGYFDN (SEQ ID NO:23); and/or ii) a variable region comprising a first CDR comprising SSVSY (SEQ ID NO:81) or SSSVSY (SEQ ID NO:28) operably linked to a second CDR comprising DTS (SEQ ID NO:29) operably linked to a third CDR comprising QQWSSNPPT (SEQ ID NO:30); or iii) a variable region comprising a first CDR comprising GGSISSYY (SEQ ID NO:35) operably linked to a second CDR comprising ISTSGFT (SEQ ID NO:36) operably linked to a third CDR comprising ARDGWGRVFDI (SEQ ID NO:37); and iv) a variable region comprising a first CDR comprising QSVSSSY (SEQ ID NO:42) operably linked to a second CDR comprising GAS (SEQ ID NO:43) operably linked to a third CDR comprising QQYGSST (SEQ ID NO:44).

In one embodiment, an isolated nucleic acid is provided comprising a promoter, e.g., a heterologous promoter, operably linked to a nucleotide sequence which encodes at least the variable region of a heavy or light Ig chain that binds human and/or mouse ART1, wherein the chain comprises: i) a variable region comprising a first complementarity determining region (CDR) comprising GFSLSNARM (SEQ ID NO:66) or NARMGVS (SEQ ID NO:21) operably linked to a second CDR comprising IFSNDEK (SEQ ID NO:67) or HIFSNDEKSYSTSLKS (SEQ ID NO:22) operably linked to a third CDR comprising ARIYGGDSWGYFDN (SEQ ID NO:68) or IYGGDSWGYFDN (SEQ ID NO:23); and/or
ii) a variable region comprising a first CDR comprising SSVSY (SEQ ID NO:81) or SSSVSY (SEQ ID NO:28) operably linked to a second CDR comprising DTS (SEQ ID NO:29) operably linked to a third CDR comprising QQWSSNPPT (SEQ ID NO:30); or iii) a variable region comprising a first CDR comprising GGSISSYY (SEQ ID NO: 35) operably linked to a second CDR comprising ISTSGFT (SEQ ID NO:36) operably linked to a third CDR comprising ARDGWGRVFDI (SEQ ID NO:37); and/or iv) a variable region comprising a first CDR comprising QSVSSSY (SEQ ID NO:42) operably linked to a second CDR comprising GAS (SEQ ID NO:43) operably linked to a third CDR comprising QQYGSST (SEQ ID NO:44).

An isolated antibody or antigen fragment thereof that binds human and mouse ART1 is provided, wherein the antibody or the antigen binding fragment thereof have: i) a variable region comprising a first complementarity determining region (CDR) comprising GFSLSNARM (SEQ ID NO:66) or NARMGVS (SEQ ID NO: 21) operably linked to a second CDR comprising IFSNDEK (SEQ ID NO:67) or HIFSNDEKSYSTSLKS (SEQ ID NO:22) operably linked to a third CDR comprising ARIYGGDSWGYFDN (SEQ ID NO:68) or IYGGDSWGYFDN (SEQ ID NO:23); and/or ii) a variable region comprising a first CDR comprising SSVSY (SEQ ID NO:81) or SSSVSY (SEQ ID NO:28) operably linked to a second CDR comprising DTS (SEQ ID NO: 29) operably linked to a third CDR comprising QQWSSNPPT (SEQ ID NO:30); iii) a variable region comprising a first CDR comprising GGSISSYY (SEQ ID NO:35) operably linked to a second CDR comprising ISTSGFT (SEQ ID NO:36) operably linked to a third CDR comprising ARDGWGRVFDI (SEQ ID NO:37); and/or iv) a variable region comprising a first CDR comprising QSVSSSY (SEQ ID NO:42) operably linked to a second CDR comprising GAS (SEQ ID NO:43) operably linked to a third CDR comprising QQYGSST (SEQ ID NO: 44). In one embodiment, the variable region of i) in the antibody or fragment thereof further comprises one or more framework regions comprising one or more of: QVTLKESGPVLVKPTETLTLTCTVSGFSLS (SEQ ID NO:24), WIRQPPGKALEWLA (SEQ ID NO:25), RLTISKDTSKSQVVLTMTNMDPVDTATYYCAR (SEQ ID NO:26), WGQGTLVTVSS (SEQ ID NO:27), QVTLKESGPVLVKPTETLTLTCTVS (SEQ ID NO:71), VSWIRQP PGKALEWLAH (SEQ ID NO:72), SYSTSLKSRLTISKDTSKSQVVLTMTNMDPVDTATYYC (SEQ ID NO:73), or WGQGTLVTVSS (SEQ ID NO: 74). In one embodiment, the variable region of ii) of the anibody or fragment thereof further comprises one or more framework regions comprising one or more of: QIVLTQSPAIMSASLGEKVTMTCSA (SEQ ID NO:31), MHWYQQKSGTSPKRWIY (SEQ ID NO:32), KLASGVPARFSGSGSGTSYSLTISSMEAGDAATYYC (SEQ ID NO:33), FGAGTKLELK (SEQ ID NO:34), DIQLTQSPSFLSASVGDRVTITCRAS (SEQ ID NO:76), YMHWYQQKPGTS PKRLIY (SEQ ID NO:77), KLASGVPSRFSGSGSGTEYTLTISSLOPEDFATY YC (SEQ ID NO:78), FGQGTKLEIK (SEQ ID NO:79). In one embodiment, the variable region of ii) further comprises one or more framework regions comprising one or more of: DIQLTQSPSFLSASVGDRVTITCRA (SEQ ID NO:51), MHWYQQKPGTSPKRLIY (SEQ ID NO:52), KLASGVPSRFSGSGSGTEYTLTISSLQPEDFATYYC (SEQ ID NO:53), or FGQGTKLEIKRTVAAPSVFIFPPSDEQLKSGTASVVCLLNNFYPREAKVQWKVDNALQSGNSQESVTEQDSK DSTYSLSSTLTLSKADYEKHKVYACEVTHQGLSSPVTKSFNRGEC (SEQ ID NO:54). In one embodiment, the variable region of ii) further comprises one or more framework regions comprising one or more of:
DIQMTQSPSSLSASVGDRVTITCSA (SEQ ID NO:55), MHWYQQKPGTSPKRLIY (SEQ ID NO:56), KLASGVPSRFSGSGSGTDYTLTISSLQPEDFATYYC (SEQ ID NO:57), or FGQGTKLEIKRTVAAPSVFIFPPSDEQLKSGTASVVCLLNNFYPREAKVQWKVDNALQSGNSQESVTEQDSK DSTYSLSSTLTLSKADYEKHKVYACEVTHQGLSSPVTKSFNRGEC (SEQ ID NO:58). In one embodiment, the variable region of ii) further comprises one or more framework regions comprising one or more of: EIVLTQSPATLSLSPGERATLSCRA (SEQ ID NO:59), MHWYQQKPGTSPRRLIY (SEQ ID NO:60), KLATGIPARFSGSGSGTDYTLTISSLEPEDFAVYYC (SEQ ID NO:61), or TFGQGTKLEIKRTVAAPSVFIFPPSDEQLKSGTASVVCLLNNFYPREAKVQWKVDNALQSGNSQESVTEQDS KDSTYSLSSTLTLSKADYEKHKVYACEVTHQGLSSPVTKSFNRGEC (SEQ ID NO:62). In one embodiment, the variable region of iii) further comprises one or more framework regions comprising one or more of: QVQLQESGPGLVKPSETLSLTCTVS (SEQ ID NO:38), WSWIRQPAGKGLEWIGR (SEQ ID NO:39), NYNPSLKSRVTMSVDSSKNQFSLKLSSLTAADTAVYYC (SEQ ID NO:40), or WGLGTMVTVSS (SEQ ID NO: 41). In one embodiment, the variable region of iv) further comprises one or more framework regions comprising one or more of: EIVLTQSPGTLSLSPGERATLSCRAS (SEQ ID NO:45), LAWYQQKPGQAPRLLIY (SEQ ID NO:46), SRATGIPDRFSGSGSGTDFTLTISRLEPEDFAVYYC (SEQ ID NO:47) or FGPGTKVDIK (SEQ ID NO:63).

Also provided is a method to inhibit or treat cancer in a mammal, comprising: administering to a mammal a composition comprising an effective amount of an anti-human ART1 antibody or an antigen binding fragment thereof, or a polypeptide that binds human ART1. In one embodiment, the cancer is lung cancer, colon cancer, melanoma, glioblastoma, breast cancer or colorectal cancer. In one embodiment, the mammal is a human. In one embodiment, the amount is effective to decrease tumor burden, inhibit metastases, increase survival, or any combination thereof. In one embodiment, the composition is intravenously or subcutaneously administered. In one embodiment, the method further comprises administering a chemotherapeutic drug. In one embodiment, the method further comprises administering an immune checkpoint inhibitor. In one embodiment, the antibody, the antigen binding fragment thereof, or the polypeptide, has:
i) a variable region comprising a first complementarity determining region (CDR) comprising GFSLSNARM (SEQ ID NO:66) or NARMGVS (SEQ ID NO:21) operably linked to a second CDR comprising IFSNDEK (SEQ ID NO:67) or HIFSNDEKSYSTSLKS (SEQ ID NO:22) operably linked to a third CDR comprising ARIYGGDSWGYFDN (SEQ ID NO:68) or IYGGDSWGYFDN (SEQ ID NO:23); and/or
ii) a variable region comprising a first CDR comprising SSVSY (SEQ ID NO:81) or SSSVSY (SEQ ID NO:28) operably linked to a second CDR comprising DTS (SEQ ID NO:29) operably linked to a third CDR comprising QQWSSNPPT (SEQ ID NO:30); or iii) a variable region comprising a first CDR comprising GGSISSYY (SEQ ID NO: 35) operably linked to a second CDR comprising ISTSGFT (SEQ ID NO:36) operably linked to a third CDR comprising ARDGWGRVFDI (SEQ ID NO:37); and/or iv) a variable region comprising a first CDR comprising QSVSSSY (SEQ ID NO:42) operably linked to a second CDR comprising GAS (SEQ ID NO:43) operably linked to a third CDR comprising QQYGSST (SEQ ID NO:44). In one embodiment, the heavy chain is an IgG heavy chain. In one embodiment, the light chain is an Igk light chain. In one embodiment, the antibody fragment is administered. In one embodiment, the fragment is Fab′ or scFv.

Further provided is a method to prevent, inhibit or treat ART1-mediated immunosuppression in a mammal, comprising: administering to a mammal a composition comprising an effective amount of an anti-human ART1 antibody or an antigen binding fragment thereof, or a polypeptide that binds human ART1. In one embodiment, the mammal has cancer. In one embodiment, the mammal is a human. In one embodiment, the composition is intravenously administered. In one embodiment, the composition is subcutaneously administered In one embodiment, the method further comprises administering a chemotherapeutic drug. In one embodiment, the method further comprises administering an immune checkpoint inhibitor. In one embodiment, the antibody, the antigen binding fragment thereof, or the polypeptide has:
i) a variable region comprising a first complementarity determining region (CDR) comprising GFSLSNARM (SEQ ID NO:66) or NARMGVS (SEQ ID NO:21) operably linked to a second CDR comprising IFSNDEK (SEQ ID NO:67) or HIFSNDEKSYSTSLKS (SEQ ID NO:22) operably linked to a third CDR comprising ARIYGGDSWGYFDN (SEQ ID NO:68) or IYGGDSWGYFDN (SEQ ID NO:23); and/or
ii) a variable region comprising a first CDR comprising SSVSY (SEQ ID NO:81) or SSSVSY (SEQ ID NO:28) operably linked to a second CDR comprising DTS (SEQ ID NO:29) operably linked to a third CDR comprising QQWSSNPPT (SEQ ID NO:30); or iii) a variable region comprising a first CDR comprising GGSISSYY (SEQ ID NO: 35) operably linked to a second CDR comprising ISTSGFT (SEQ ID NO:36) operably linked to a third CDR comprising ARDGWGRVFDI (SEQ ID NO:37); and/or iv) a variable region comprising a first CDR comprising QSVSSSY (SEQ ID NO:42) operably linked to a second CDR comprising GAS (SEQ ID NO:43) operably linked to a third CDR comprising QQYGSST (SEQ ID NO:44). In one embodiment, the heavy chain is an IgG heavy chain. In one embodiment, the light chain is an Igκ light chain.

A method to enhance an immune response in a mammal having cancer is provided comprising: administering to a mammal a composition comprising an effective amount of an anti-human ART1 antibody or an antigen binding fragment thereof, or a polypeptide that binds human ART1. In one embodiment, the mammal is a human. In one embodiment, the heavy chain is an IgG heavy chain. In one embodiment, the light chain is an Igκ light chain. In one embodiment, the antibody, the antigen binding fragment thereof, or the polypeptide has:
i) a variable region comprising a first complementarity determining region (CDR) comprising GFSLSNARM (SEQ ID NO:66) or NARMGVS (SEQ ID NO:21) operably linked to a second CDR comprising IFSNDEK (SEQ ID NO:67) or HIFSNDEKSYSTSLKS (SEQ ID NO:22) operably linked to a third CDR comprising ARIYGGDSWGYFDN (SEQ ID NO:68) or IYGGDSWGYFDN (SEQ ID NO:23); and/or
ii) a variable region comprising a first CDR comprising SSVSY (SEQ ID NO:81) or SSSVSY (SEQ ID NO:28) operably linked to a second CDR comprising DTS (SEQ ID NO:29) operably linked to a third CDR comprising QQWSSNPPT (SEQ ID NO:30); or iii) a variable region comprising a first CDR comprising GGSISSYY (SEQ ID NO: 35) operably linked to a second CDR comprising ISTSGFT (SEQ ID NO:36) operably linked to a third CDR comprising ARDGWGRVFDI (SEQ ID NO:37); and/or iv) a variable region comprising a first CDR comprising QSVSSSY (SEQ ID NO:42) operably linked to a second CDR comprising GAS (SEQ ID NO:43) operably linked to a third CDR comprising QQYGSST (SEQ ID NO:44).

In one embodiment, an isolated antibody or fragment thereof is provided, wherein, when bound to human ART1, the antibody binds to at least one of the following residues: S75, S77, T79, R80, R89, H92, or Y99 of human ART1, e.g., SEQ ID NO:90. In one embodiment, an isolated antibody or fragment thereof binds to two, three, four, five or six of S75, S77, T79, R80, R89, H92, or Y99 of human ART1, e.g., SEQ ID NO: 90. In one embodiment, an isolated antibody or fragment thereof binds to S75, S77, T79, R80, R89, H92, and Y99 of human ART1, e.g., SEQ ID NO:90.

### BRIEF DESCRIPTION OF THE FIGURES

FIG. 1. huART1 biochemical enzymatic assay with various MAbs.

FIG. 2. huART1 cell based enzymatic assay with various MAbs.

FIG. 3. huART1/moART1 biochemical enzymatic assay with various MAbs.

FIG. 4. huART1 binding characterization of 12F09 and 14G01.

FIG. 5. moART1 binding characterization of 12F09 and 14G01.

FIG. 6. Purified 14G01 and 22C12 characterization.

FIG. 7. 22C12 binding KP-1−/+Dox.

FIG. 8. 14G01 binding KP-1−/+Dox.

FIG. 9. 22C12 binding KP-1 OE−/+Dox.

FIG. 10. 14G01 binding KP-1 OE−/+Dox.

FIGS. 11A-11C. Identification of Functional ART1 Antibodies. A) Anti-human ART1 positive wells were tested for inhibition of purified human ART1 using a fluorescent readout that measures NAD⁺. B) Anti-human ART1 wells were tested for inhibition of human ART1 expressed transiently in HEK293 cells by cell-surface ADP-ribosylation assay. C) Clones 22C12 and 14G01 were tested for inhibition of purified mouse ART1. Anti-ART1 hybridoma supernatants were also tested for inhibition of human ART1 transiently expressed in HEK293 cells. This method, which has previously been described for ART2 (Krebs et. al.), determines cell-surface ADP-ribosylation via an NAD⁺ analogue (etheno-NAD⁺) which is then detected by an anti-etheno antibody using flow cytometry. Two hybridoma supernatants (fusion wells 22C12 and 14G01) were positive for inhibition of human ART1 in the biochemical and cell-based assays. Supernatants from 22C12 and 14G01 were then tested for binding to moART1 by ELISA and inhibition of purified mouse ART1.

FIGS. 12A-12D. Affinity Measurement of 22C12 and 14G01 to Human and Mouse ART1 by Surface Plasmon Resonance. Purified antibodies from hybridoma clones 22C12 and 14G01 were captured on an anti-Mouse Fc surface and the indicated concentration of analyte was injected over the surface. A) 22C12 binding to human ART1. B) 22C12 binding to mouse ART1. C) 14G01 binding to human ART1. D) 14G01 binding to mouse ART1. Sensorgrams are double-reference subtracted using a control surface and blank injections. Affinity constants were determined by kinetic fit using a 1:1 binding model.

FIG. 13. Dose-Dependent Inhibition of Cell-Surface ADP-Ribosylation by 22C12 and 14G01. HEK293 cells transiently transfected with human ART1 were incubated with 22C12 or 14G01 at the indicated concentration followed by treatment with etheno-NAD⁺, staining with anti-etheno antibody and flow cytometry. % Inhibition was normalized to HEK293 cells transfected with human ART1 and stained with anti-etheno antibody without etheno-NAD⁺ treatment.

FIGS. 14A-14G. ART1 is overexpressed in a subset of human lung cancers. (A-B) ART1 Immunofluorescence staining of human lung cancer cell lines A549 and H1650 and benign bronchial epithelium cell line BEAS2B. A) Representative pictures of ART1 surface expression in non-permeabilized cells (left column) and ART1 total cell expression in permeabilized cells (right column). B) Mean fluorescence intensity (MFI) of ART1 surface staining on A549, H1650 and BEAS2B cells. Each dot represents the ART1 MFI of one cell. C) Ratio of ART1 surface MFI and ART1 total MFI (graph in FIG. 19A) in BEAS2B, A549 and H1650 cells (n=1) D) Violin plot depicting ART1 qPCR analysis of matched lung tumor tissue and normal lung tissue from patients with stage I-III lung adenocarcinoma (n=40), Wilcoxon matched pairs signed rank test. (Median is indicated by dotted red line, quartiles are indicated by solid red lines) (E-F) IHC analysis of ART1 expression in a human tissue microarray (TMA) containing 493 stage I adenocarcinomas. Localization of ART1 expression was scored as (1) membranous (with or without cytoplasmic staining) or (2) tumor cell cytoplasm only. Tumors were scored for infiltration of immune cells (listed in Table 4). E) Representative IHC images and pie chart depicting percentage of tumors that stained positive for ART1 membranous expression or ART1 expression in the cytoplasm only (F) Percentage of tumors with a low or intermediate/high CD8 T cell score in tumors with membranous ART1 staining or cytoplasmic ART1 staining only. Chi-square test. Gene expression data were square root-transformed prior to statistical testing. * p<0.05. G) mRNA expression analysis of ART1 gene expression as well as genes associated with CD8 T cell cytotoxicity; IFNgamma (IFNG), Granzyme A (GZMA), Granzyme B (GZMB), Perforin 1 (PRF1) 41BB (TNFRSF9) and immunoregulatory genes; CTLA-4 (CTLA4), PD-1 (PDCD1), Tim-3 (HAVCR2), Lag-3 (LAG3) and Tigit (TIGIT) in lung adenocarcinoma patients from the TCGA PanCancer Atlas cohort (n=503). Clustered OncoPrint heatmap depicting mRNA expression z-scores relative to all samples.

FIGS. 15A-15D. ART1 expression promotes tumor growth in murine lung tumor models. A) Ectopic subcutaneous (s.c.) flank tumor model assessing growth of KP1-ART1^{OE} tumors in wild type and immunodeficient nude mice. (B-D) Orthoptopic lung tumor model assessing lung tumor burden and lung infiltration of CD8 T cells. In both the ectopic and orthotopic model, KP1 cells were stably transduced with ART1 overexpression lentiviral vector (KP1-ART1^{OE}) and subsequently transduced with shRNA targeting ART1 (shART1). Where indicated mice were treated with doxycycline-water ad libitum to induce shART1. A) Growth curves of subcutaneous KP1-ART1^{OE} flank tumors in immunocompetent wild type C57BL/6 mice (left panel, n=5 mice/group) and in immunodeficient athymic nude mice (right panel, n=5 mice/group). Statistically significant differences in tumor growth between groups was determined by repeated-measures ANOVA. B) Experimental schema for orthotopic lung tumor model. C) Representative images of lung sections stained with H&E (left panels) and enumeration of lung nodules (right panel) from mice sacrificed on day 14 after injection of KP1-ART1^{OE}. (n=4-5 mice/group) Welch's t-test. Lung nodules in H&E stainings are indicated by black arrows. Tumor nodule counts were determined using Image J software. D) Percentage of CD8 T cells among total lung-infiltrating leukocytes (CD45+ cells) at day 16 and 25 after tumor injection. Welch's t-test. Box and whiskers plots indicate median and 10-90 percentiles. Percentage and count data were square root-transformed prior to statistical testing ** p<0.01

FIGS. 16A-16L. ART1 blockade reduces lung tumor burden and promotes infiltration of P2X7R+ CD8 T cells (A-H) In vivo experiment studying lung tumor burden and lung immune cell analysis in mice orthotopically inoculated by tail vein injections with KP1-ART1^{OE} tumors. Mice received intraperitoneal (i.p.) treatment with ART1 blocking antibody (22C12 Ab) or isotype matched control antibody (iso ctrl Ab) every three days starting on day 6 until day 18 (n=7-8 mice/group). The experiment was repeated once with similar results A) Experiment schema B) Representative H&E staining images of sections of mouse lungs. C) Average lung tumor nodule counts and (D) average lung nodule area on day 19 after tumor inoculation. Welch's t-test. (E-H) Immune phenotyping by flow cytometry of digested mouse lungs on day 19 following treatment with 22C12 Ab or iso ctrl Ab. E) Representative dot plots and box plots showing the percentage of CD8 T cells expressing P2X7R and/or the proliferation marker Ki67. F) Representative dot plots and box plots showing the percentage of CD8 T cells expressing P2X7R and/or the immunoregulatory receptor PD-1. (G-H) Absolute counts of CD8 T cell subsets normalized to lung weight in the KP1-ART1^{OE} lung tumor model on day 25 after tumor cell injection (n=6-7). G) Counts of P2X7R+ CD8 T cells, PD-1+ CD8 T cells and Ki67high CD8 T cells per gram of tumor-bearing lung tissue. (H) Counts of P2X7R+ CD8 TCM, P2X7R+ CD8 TEM, and P2X7R+ CD8 TRM per gram of tumor-bearing lung tissue. Box and whiskers plots indicate median and 10-90 percentiles. Welch's t-test. Percentage and count data were square root transformed prior to statistical testing. (I-L) In vivo experiment studying the effect of CD8 T cells and CD4 T cell depletion on the anti-tumor effect of ART1 blockade. Mice were orthotopically inoculated by tail vein injections with KP1-ART1^{OE} tumors on day 0. Where indicated, mice received intraperitoneal treatment with ART1 blocking antibody (22C12 Ab) or isotype matched control antibody (iso ctrl Ab) (25 mg/kg), CD8 depleting antibodies (clone: 53-6.7) or CD4 depleting antibodies (clone: GK1.5) 500 ug on day−1 followed by 250 ug every three days from day 3-18 (n=7-8 mice/group). I) Experimental schema (J) Representative H&E staining images of sections of mouse lungs (K) average lung tumor nodule counts and (L) average lung nodule area on day 19 after tumor inoculation. Tumor nodule counts and area were determined using Image J software.a . . . u.=arbitrary units. Box and whiskers plots indicate median and 10-90 percentiles. One-way ANOVA. Percentage and count data were square root-transformed prior to statistical testing *p<005, ** p<0.01, *** p<0.001

FIGS. 17A-17C. ART1-mediated ADP-ribosylation and NICD of lung tumor-infiltrating T cell subsets (A-C) ADP-ribosylation and NICD assay. T cells isolated from wild type KP1 tumor bearing lungs of C57BL/6 mice were incubated for two hours with ethano-NAD (eNAD) alone (-rART1) or with eNAD and recombinant mouse ART1 (rART1), CD38 blocking antibody (NIMR-5) (CD38 block) or ART1 blocking antibody (ART1 block (22C12)). In order to exclusively measure ART1-blockade, ART2 activity was blocked in all culture conditions using the ART2-blocking nanobody (s+16a). After co-culture, T cells were analyzed by flow cytometry for ADP-ribosylation by eNAD staining and for cell death by DAPI staining (n=7). A) Example gating depicting identification of CD8 T cells, CD4 Tconv (CD4+ CD25−) and CD4 Tregs (CD4+ CD25+). The P2X7R+ and P2X7R− fractions of each T cell subset were analyzed separately for (B) ADP-ribosylation by total eNAD staining and (C) NICD based on co-staining with eNAD and DAPI. Repeated measures one-way ANOVA was used to determine statistically significant differences between treatments. Each connected line represents paired analysis of one mouse. Percentage data were square root-transformed prior to statistical testing *p<0.05, ** p<0.01. *** p<0.001, **** p<0.0001

FIGS. 18A-18H. ART1 overexpression in human lung tumors is associated with low infiltration of P2X7R+ CD8 T cells. (A-E) Analysis of immunofluorescence stainings of lung tumor tissue and matched normal lung tissue from lung adenocarcinoma patients (n=12). A) Representative images of ART1 immunofluorescence staining in lung tumor and matched normal tissue. B) Bar graph depicting mean fluorescence intensity (MFI) of ART1 staining normalized to DAPI MFI in lung tumor and matched normal tissue. Paired t-test. C) Representative images of CD8 (red), P2X7R (green) and nuclear stain by DAPI (blue) immunofluorescence staining in lung tumor and matched normal tissue. Yellow color in the merged images indicate co-localization of CD8 and P2X7R staining (highlighted by white arrows). D) Bar graph depicting percentage of P2X7R+ CD8 T cells among CD8 T cells in lung tumor and matched normal tissue. Paired t-test. E) Linear regression analysis correlating percent change of P2X7R+ CD8 T cells in lung tumor from matched tissue with percent change in ART1 MFI in lung tumor from matched normal tissue. R2 represents Pearson correlation coefficient. (F-H) Flow cytometry analysis of CD8 T cells infiltrating lung tumor tissue and adjacent matched normal lung tissue from lung adenocarcinoma patients (n=5). F) Representative dot plot of P2X7R and CD38 expression on CD8 T cells infiltrating normal lung tissue and lung tumor tissue. G) Percentage of CD8 T cells expressing P2X7R. Paired t-test (H) Percentage of P2X7R+ CD8 T cells with high surface expression of CD38. Bars graphs indicate mean values. Paired t-test. Percentage and MFI data were log-transformed prior to statistical testing *p<005, *** p<0.001, **** p<0.0001

FIGS. 19A-19D. ART1 and GPLD1 expression in human lung cancer cell lines and tumor samples, Expression of cytotoxicity and immunoregulatory genes in lung adenocarcinoma patients (TCGA). A) Immunofluorescence surface staining of total ART1 expression in permeabilized human lung cancer cells A549, H1650 and benign bronchial epithelial cell line BEAS2B. Each dot represents the ART1 MFI of one cell. B) Violin plot depicting GPLD1 qPCR analysis of matched lung tumor tissue and normal lung tissue from patients with stage I-III lung adenocarcinoma (n=40), Wilcoxon matched pairs signed rank test. (Median is indicated by dotted red line, quartiles are indicated by solid red lines). C) IHC analysis of ART1 expression in a human tissue microarray (TMA) containing 493 stage I adenocarcinomas. Representative IHC images and pie chart depicts percentage of tumors that had weak, moderate or strong ART1 staining. D) mRNA expression analysis of ART1 gene expression as well as genes associated with CD8 T cell cytotoxicity; IFNγ (IFNG), Granzyme A (GZMA), 41BB (TNFRSF9) and immunoregulatory genes; CTLA-4 (CTLA4), PD-1 (PDCD1), and Tigit (TIGIT) in lung adenocarcinoma patients from the TCGA PanCancer Atlas cohort (n=503). Box plots depict expression of genes that showed statistically significant differences between lung adenocarcinoma patients with high ART1 tumor expression (ART1^{high}, z-score>1, n=115) or low ART1 tumor expression (ART1^{OW}, z-score<1 n=395). Box and whiskers plots indicate median and 25-75 percentiles. ** p<0.01

FIGS. 20A-20D. ART1 overexpression and knockdown in KP1 cells A) Representative images of ART1 surface Immunofluorescence staining and quantification of ART1 mean fluorescence intensity (MFI) in KP1 and KP1ART1OE cells with and without doxycycline induction of shART1 as well as Lewis Lung Carcinoma (LLC1) cells. Scatter plot shows mean with SEM. Each dot represents the ART1 MFI of one cell. B) MAR/PAR immunofluorescence staining measuring ADP-ribosylation of KP1-ART1OE cells with and without doxycycline induction of shART1 in the presence or absence of NAD+ (20 μM). C) Proliferation assay testing the growth of KP1-ART1OE cells in vitro with or without doxycycline-induction of shART1. D) ART1 immunofluorescence staining of KP1-ART1OE tumors harvested on day 31 after tumor inoculation from mice with or without doxycycline induction of shART1. Graph depicts mean fluorescence intensity (MFI) of ART1 staining normalized to Hoechst MFI. Box and whiskers plots indicate median and 10-90 percentiles. ART1 MFI values were square-root transformed. Welch's t-test. * p<0.05, **** p<0.0001

FIGS. 21A-21D. CRISPR/Cas9-mediated knockout of ART1 in B16-F10 cells (A-B) Confirmation of ART1 knockout in CRISPR/Cas9 clones of B16-F10 mouse melanoma cells. B16-F10 scramble clone B16CONTROL (Scr-6) was transduced with a non-specific CRISPR gRNA and serves as a control. The B16-F10 ART1 knockout (ART1KO) clones B16ART1KO (42-1) and B16ART1KO (63-1) were transduced with gRNA targeting two different regions in exon 1 of the ART1 gene. A) Flow cytometry cell surface staining of ART1 on B16 CRISPR clones B16CONTROL (Scr-6), B16ART1KO (42-1) and B16ART1KO (63-1). Orange histograms represent cells stained with ART1 primary antibody (rabbit IgG) and Cy5-conjugated secondary anti-rabbit antibody. Blue histograms represents cells stained with secondary antibody alone. B) Immunofluorescence staining of ART1 in vitro of B16 control clone B16CONTROL (Scr-6) and B16 ART1 knockout clones B16ART1KO (42-1) and B16ART1KO (63-1). C) Flank tumor growth of B16 control clone B16CONTROL (Scr-6) and B16 ART1 knockout clones B16ART1KO (42-1) and B16ART1KO (63-1) in wild type C57BL/6 mice (n=5-6 mice/group). Mice were injected with 1×10⁵ cells of the respective CRISPR clone in the right flank (n=6 mice/group), Repeated-measures ANOVA from day of tumor inoculation until Day 21, p-values indicate statistically significant difference in tumor growth compared with B16CONTROL (Scr-6). D) Proliferation assay testing the growth of B16 CRISPR clones B16CONTROL (Scr-6), B16ART1KO (42-1) and B16ART1KO (63-1) in vitro. Red stars and blue stars represent statistically significant difference in cell number at the indicated time points between B16CONTROL (Scr-6) and B16ART1KO (63-1) or B16ART1KO (42-1) respectively. Student's t-test *p<0.05, ** p<0.01, *** p<0.001.

FIGS. 22A-22H. Generation and functional testing of ART1-blocking antibody 22C12. A) Schematic representation of the workflow for generation of ART1-blocking antibody 22C12. B) Screening of ART1-binding antibody clones by NAD-Glo assay showing inhibition of ART1 activity by clone 22C12. C) NAD-Glo assay determining half-maximum binding (EC50) values of 22C12 antibody clones with mouse light chains (22C12 (mLC)) and human light chains (22C12 (HuLC) against HEK293 cells transfected with ART1. D) Flow cytometry histogram depicting binding of 22C12 antibody (mLC) to KP1-ART1OE cells. E) Etheno-NAD (eNAD) ADP-ribosylation assays determining half-maximum inhibition (IC50) of ADP-ribosylation of 22C12 antibody clones, against HEK293 cells transfected with ART1. Hu IgG1 was used as isotype control. 2E2 antibody was used as an irrelevant antibody. F) Western blot analysis of ADP-ribosylated proteins (using MAR/PAR antibody) in KP1-ART1OE cells incubated for 2 hours with indicated concentrations of NAD+ treated with or without 20 ug/ml of 22c12 ART1 antibody. (G-H) Toxicity study. Tumor-naïve mice were treated with intraperitoneal injections of 22C12 antibodies at 25 mg/kg every three days for three weeks and monitored for weight loss and blood glucose levels at baseline and every week until the end of the study. Mice treated with 22c12 antibodies remained normal in appearance, activity, gait and alertness compared to mice treated with isotype control antibody. G) Mouse weight depicted as fold change from baseline weight at day 0. H) Mouse plasma glucose levels depicted as fold change from baseline measurement at day 0.

FIGS. 23A-23G. In vivo inhibition of ART1 reduces tumor burden and promotes P2X7R+ CD8 T cell tumor infiltration in KP1-ART1OE and LLC1 tumor models. A) Experiment schema for in vivo experiment studying tumor progression of KP1-ART1OE flank tumors following intratumoral (i.t.) treatment with anti-ART1 Ab (22C12 Ab) or IgG2A isotype control Ab (iso ctrl Ab) (n=7-8 mice/group). Tumors were harvested on day 25 after tumor inoculation for weighing and flow cytometry analysis. The Experiment was repeated once with similar results. B) Growth of subcutaneous KP1-ART1OE flank tumors treated with i.t. injections of 22C12 Ab or iso ctrl Ab. Repeated-measure ANOVA mixed effects model (C) Tumor weight of excised tumors treated with 22C12 antibody or iso ctrl antibody on day 25 after tumor inoculation. Welch's t-test. (D-F) LLC1 orthotopic lung tumor model. Mice were inoculated with 1.5×10⁵ LLC1 cells by intravenous injection on day 0. Intraperitoneal treatment with 22C12 Ab or iso ctrl Ab (25 mg/kg, n=9 mice/group) was delivered every three days starting on day 6 until day 21. On day 22, mice were sacrificed, and lungs were fixated and stained with H&E to determine lung tumor burden. Infiltration of P2X7R+ CD8 T cells in tumor-bearing lungs was assessed by flow cytometry analysis. D) Average lung tumor nodule counts and (E) average lung nodule area on day 22 after tumor inoculation. Tumor nodule counts and area was determined using Image J software. a.u.=arbitrary units. Welch's t-test. F) Flow cytometry analysis of frequency of P2X7R+ CD8 T cells among total CD8 T cells infiltrating LLC1-bearing mouse lungs on day 22 after tumor inoculation. G) Flow cytometry analysis of KP1-ART1OE-bearing mouse lungs assessing frequency of P2X7R+CD8 T cells among total CD8 T cells in mice on day 18 after tumor injection/shART1 induction. (n=6 mice/group). Welch's t-test. Box and whiskers plots indicate median and 10-90 percentiles. Percentage data was square root-transformed prior to statistical testing. * p<0.05, ** p<0.01.

FIGS. 24A-24B. CD8/CD4 T cell depletion study in B16-ARTKO flank tumor model (A-B) In vivo experiment studying the effect of CD8 T cells and CD4 T cell depletion on the progression of ART1-proficient and ART1-deficient B16-F10 flank tumors. Wild type C57BL/6 mice were ectopically inoculated by flank injections of 1λ10⁵ B16CONTROL (clone Scr-6) or B16ART1KO (clone 63-1) tumor cells on day 0 (n=7-8 mice/group). Mice were treated with CD8 depleting antibodies (CD8 depl, clone: 53-6.7), or CD4 depleting antibodies (CD4 depl, clone: GK1.5), or isotype control antibodies (iso ctrl). 500 ug on day-2 followed by 250 ug every five days from day 2 until endpoint. A) Flank tumor growth curves of control B16Scr tumors (upper panels) and ART1 knockout B16ART1KO tumors (lower panels) Asterisks in graphs indicate number of tumor free mice at endpoint. B) Kaplan-Meier plot showing percent survival of mice. Log-Rank (Mantel-Cox) test. *p<0.05, *** p<0.001.

FIGS. 25A-25C. RNAseq analysis of P2X7R expression and immunoregulatory genes in CD8 T cells from KP1 lung-tumor bearing mice, ART1 qPCR analysis of CD8 and CD4 T cells and B16-F10 and KP1-ART1^{OE} tumor cells (A-B) RNA sequencing of CD8 T cells isolated from spleens and lungs of naïve mice and mice inoculated intravenously with KP1 tumor cells. Tumors were harvested for CD8 T cell isolation on day 7 and 17 after tumor injection. A) Heatmap depicts gene expression of P2RX7 and genes regulating CD8 T cell cytotoxicity (GZMA, GZMB, IFNG, PRF1) and immunoregulatory molecules (CTLA4, HAVCR2, LAG3, PDCD1, TIGIT). B) Scatter plot depicts gene expression of P2RX7. Kruskal-Wallis test was used to determine statistical difference between P2RX7 expression levels at indicated time points of spleen-derived and lung-derived CD8 T cells separately. Scatter plot shoes mean with SEM. C) Representative flow cytometry histogram depicting P2X7R expression on CD8 T cells infiltrating lungs from a naïve mouse or a tumor-bearing mouse on day 21 after KP1 tumor injection. * p<0.05, ** p<0.01

FIGS. 26A-26G. ADP-ribosylation test of recombinant ART1 (rART1), Inhibition of ADP-ribosylation by ART1 and ART2 blocking antibodies, P2RX7 splice variant expression in T cells and tumor cells, Proliferation of tumor cells in presence of NAD+ and ART1 blockade. A) NAD-Glo assay to measure the utilization of free NAD+ to ADP-ribosylate histone (Hist) an arginine rich substrate. ART1 was inactivated by boiling where indicated. Each dot represents a technical replicate. B) etheno-NAD (eNAD) assay measuring the blocking effect of ART1- and ART2 blockade on ADP-ribosylation of CD4 and CD8 T cells. T cells isolated from KP1 tumor bearing lungs of wild type C57BL/6 mice were incubated for two hours with ethano-NAD (eNAD) in the presence or absence of ART1-blocking antibody (22C12) or ART2 blocking nanobody (s+16a) after which they were analyzed by flow cytometry for ADP-ribosylation by eNAD staining (n=4). Box and whiskers plots indicate median and 10-90 percentiles. One-way ANOVA with Tukey's test for multiple comparisons. * p<0.05, ** p<0.01. (C-D) Gene expression analysis by qPCR depicting expression level of (C) p2rx7-k and (D) p2rx7-a isoforms in CD4 Tconv cells and CD8 T cells isolated from KP1 tumor-bearing lungs on day 15 after tumor inoculation as well as in tumor cells KP1, LLC1 and B16. (E-G) Proliferation assay testing the growth of (E) KP1ART1OE lung cells, (F) LLC1 cells and (G) B16 CRISPR clones B16CONTROL (Scr-6), B16ART1KO (42-1), B16ART1KO (63-1) in vitro in the presence of NAD+ (20 uM) and/or ART1 blocking antibody (22c12, 20 ug/ml).

FIGS. 27A-27B. A) Wild type KP1 lung carcinoma cells were exposed to Thapsigargin for 24 hours and assessed for mRNA expression of ART1 by qPCR. B) Wild type KP1 mouse lung carcinoma cells and A549 human lung carcinoma cells were exposed to radiotherapy at a single dose of 8 Gy or 20 Gy or mock-treatment (0 Gy) using the Small Animal Radiation Research Platform (SARRP). X-rays were delivered at a dose rate 271 cGy/minute. Cell surface expression of ART1 was assessed by immunofluorescence staining 48 hours after irradiation. Scatter plots shows mean with SEM. Each dot represents the ART1 MFI of one cell.

FIG. 28. CD8 T cells were isolated from wild type KP1 tumor-bearing lungs by magnetic bead sorting using CD8 (TIL) MicroBeads, mouse (Miltenyi, Catalog #130-116-478) kit. Cells were added to 48 well plates pre-coated with mouse recombinant ART1 (rART1) (10 μg/ml) for 24 hours at 4° C. 1×106 CD8 T cells were resuspended in serum-free RPMI 1640 medium (Gibco) containing 100 μM etheno-NAD and 5 μg/ml anti-ART2.2 antibody (s+16a, Biolegend, Catalog #149801) and added to wells. Cells were incubated at 37° C. for 2 hours. CD8 T cells were removed from plate by gentle pipetting and fixed with 3.7% formaldehyde for 5 minutes. Cells were spun down for 30 seconds in a microcentrifuge. The supernatant was discarded, and the cell pellet was resuspended in 1 mL of deionized H2O. The samples were then spun down for 30 seconds the pellets resuspended in 200 μL of deionized H2O. 5 μL of the cell suspension was added to each gelatin-coated slide (Gelatin-Coated Microscope Slides #1178T40, Thomas Scientific). 3 spots were made per slide and each spot was smeared with the side of a pipette tip. Slides were then placed on a hot plate and the liquid was allowed to evaporate. Each spot was demarcated with a hydrophobic barrier and air dried. Cells were immunostained by adding primary antibodies for CD8 (mouse (32-M4) #sc-1177), MAR/PAR (Poly/Mono-ADP Ribose (E6F6A) Rabbit mAb #83732), P2RX7 (Purified rat P2X7R Antibody, #148702, Biolegend) and incubated O/N in 4° C. The rest of the staining procedure was identical to the approach used for immunostaining adherent cells. The samples were then mounted using prolong gold mounting media (#P36934, Thermofisher). Slides were cold cured overnight at −20° C. in dark. Fluorescence microscopy was performed using a DMIRB inverted microscope (Leica Microsystems, Deerfield, IL), with a cooled charge-coupled device camera (Princeton Instruments, Trenton, NJ). Images were collected with a 20×1.25 numerical aperture objective. MetaMorph software (Universal Imaging, West Chester, PA) was used for image processing and quantification.

FIG. 29. Representative images of ART1 immunofluorescence staining in lung tumor and matched normal tissue from lung adenocarcinoma patients. Slides were counterstained with DAPI nuclear stain.

FIGS. 30A-301. Characterization of lung tumor-infiltrating dendritic cells (DCs) from mouse lungs orthotopically inoculated by tail vein injections with KP1-ART1OE tumor cells. Mice received intraperitoneal (i.p.) treatment with ART1 blocking antibody (22C12) or isotype matched control antibody (iso ctrl) every three days starting on day 6 until day 18. On day 19, mice were euthanized and lung were weighed and digested for flow cytometry analysis. Counting beads were added prior to acquisition to allow for quantification of absolute cell counts per gram of tumor-bearing lung tissue (n=7 mice/group). A) Frequency of DCs (CD11c+MHCII+) among total viable cells. B) Frequency of conventional type I DCs (cDC1s) (CD103+Sirpa-DCs) and (C) conventional type II DCs (cDC2s) (CD103-Sirpa+ DCs) among total DCs. D) Frequency of P2X7R+ cells among DCs, (E) cDC1s and (F) cDC2s. G) Absolute counts of lung tumor-infiltrating P2X7R+ DCs, H) P2X7R+ cDC1s and (I) P2X7R+ cDC2s normalized to lung tissue weight. Welch's t-test. Box and whiskers plots indicate median and 10-90 percentiles. Percentage data was square root-transformed prior to statistical testing. * p<0.05, ** p<0.01.

FIGS. 31A-31D. CRISPR/Cas9-mediated knockout of ART1 in B16-F10 cells and 22C12-mediated blockade of Art1 causes inhibition of murine tumor growth. Confirmation of ART1 knockout in CRISPR/Cas9 clones of B16-F10 mouse melanoma cells. B16-F10 scramble clone B16^{CONTROL (Scr-6)} was transduced with a non-specific CRISPR gRNA and serves as a control. The B16-F10 ART1 knockout (ART1KO) clone and B16^{ART1KO (63-1)} were transduced with gRNA targeting a region of exon 1 of the ART1 gene. A) Flow cytometry cell surface staining of ART1 on B16 CRISPR clones B16^{CONTROL (Scr-6)} and B16^{ART1KO (63-1)}. Orange histograms represent cells stained with ART1 primary antibody (rabbit IgG) and Cy5-conjugated secondary anti-rabbit antibody. Blue histograms represents cells stained with secondary antibody alone. B) Immunofluorescence staining of ART1 in vitro of B16 control clone B16^{CONTROL (Scr-6)} and B16^{ART1KO (63-1)} C)) Western blot analysis of ADP-ribosylated proteins (using MAR/PAR antibody) in B16^{CONTROL (Scr-6)} and B16^{ART1KO (63-1)} cells incubated for 2 hours with 20 μM NAD⁺ treated with or without 20 ug/ml anti-ART1 antibody 22C12 showing decreased MAR/PARylation with treatment with 22C12. D) Growth of subcutaneous B16^{CONTROL (Scr-6)} flank tumors treated with i.t. injections of 22C12 Ab or iso ctrl Ab and B16^{ART1KO (63-1} flank tumors treated with iso ctrl Ab, showing anti-tumor effect of 22C12 (*** p<0.001).

FIGS. 32A-32D. 22C12-mediated blockade of ART1 in LLC1 causes inhibition of murine flank tumor growth. A) Immunofluorescent surface staining of ART1 in vitro in LLC1 cells. B) Flow cytometry histogram depicting binding of 22C12 Ab to LLC1 cells in vitro. Orange histogram represents cells with 22C12+ antibody. Blue histogram represents secondary while red histogram represents isotype antibody labeled cells C) Western blot analysis of ADP-ribosylated proteins (using MAR/PAR antibody) in LLC1 cells in vitro incubated for 2 hours with 20 μM NAD⁺ treated with or without 20 ug/ml anti-ART1 antibody 22C12. The addition of NAD+ increased MAR/PARylation, which was partially blocked by 22C12. D) Growth of subcutaneous LLC1 flank tumors treated with i.t. injections of 22c12 Ab or iso ctrl Ab demonstrated anti-tumor effects of 22C12 (**** P≤0.0001).

FIG. 33. Binding of TDI-Y-009 (22C12 hLC1 IgG4) to ART1 and ART1 paralogs. Bound antibody was detected through colorimetric reaction mediated by the HRP-conjugated secondary antibody reagent.

FIG. 34. Binding of the 22C12 hLC1 IgG4 (TDI-Y-009) antibody to ART1 over-expressing KP1 cells. The binding EC50 was determined to be 4.4 nM.

FIG. 35. Plasma concentrations of TDI-Y-009 in C57BL/6 mice following single IV bolus administration. Captured antibody was measured through colorimetric reaction mediated by the HRP-conjugated secondary antibody reagent.

FIGS. 36A-36C. TDI-Y-009 suppresses tumor growth in an orthotopic lung tumor model. A) Experimental schema. B) Average lung weights. C) Nodule counts and (D) average lung nodule area on day 19 after tumor inoculation. Tumor nodule counts and area was determined using Image J software. a.u.=arbitrary units. Welch's t-test.

FIGS. 37A-37B. No adverse safety effects observed in mice treated with TDI-Y-009. A) Mouse weights depicted weekly from baseline weight at day 0. B) Mouse plasma glucose levels from baseline measurement at day 0 until week 3 following treatment.

FIG. 38. Interaction huART1 and TDI-Y-009 (SEQ ID NO: 110).

FIG. 39. ART1 expression following radiation and chemotherapy. Following treatment with radiation therapy (8 Gy×3, top row) and cisplatin (bottom row) total and surface ART1 expression increased in KP1 (left panel) and A549 cells (right panel) as measured by RTPCR and by single cell immunofluorescence.

FIG. 40. MARylation expression following radiation. In KP1 cells, in addition to increasing cell surface ART1, radiation therapy (8 Gy×3) increased cell surface mono-ADP-ribosylation (MARylation) as measured by single cell immunofluorescence using a MAR specific antibody. The increase in MARylation was blocked by the addition of 22C12 to the cell culture.

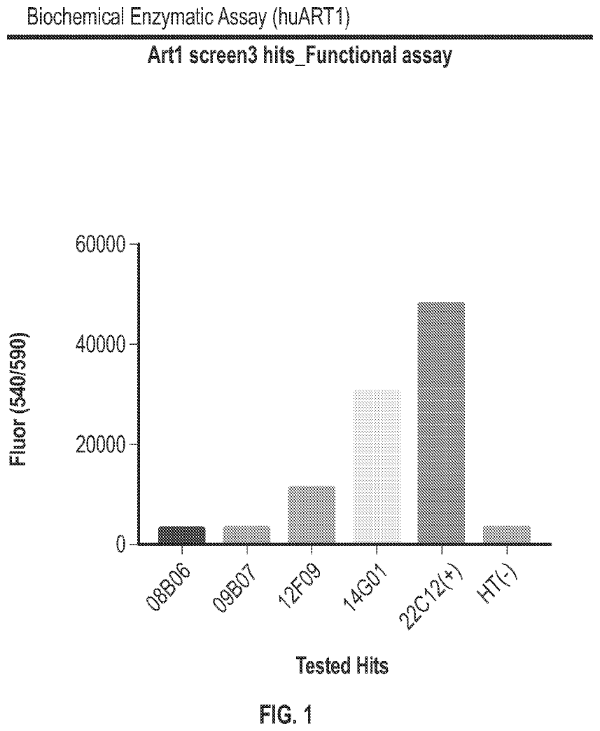

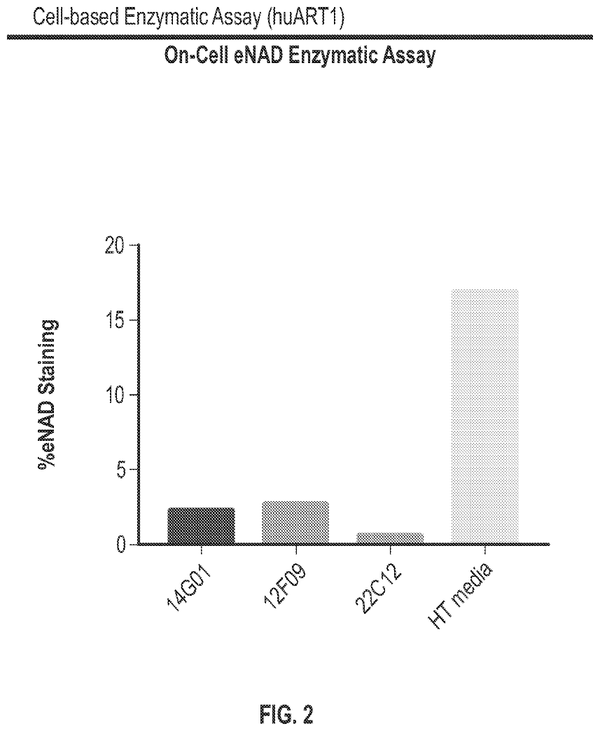

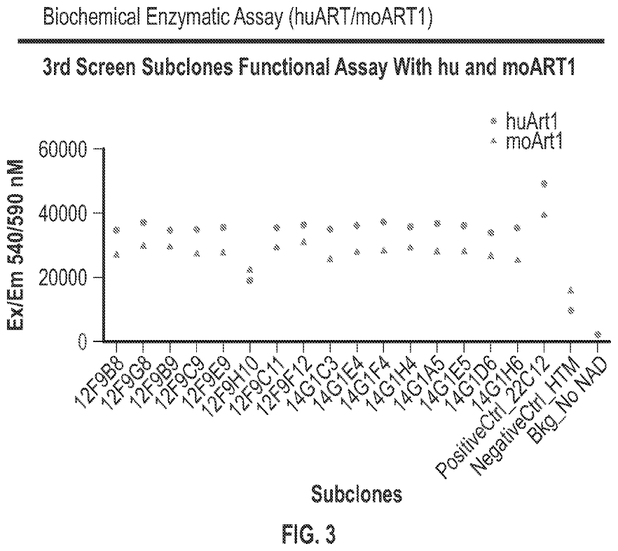

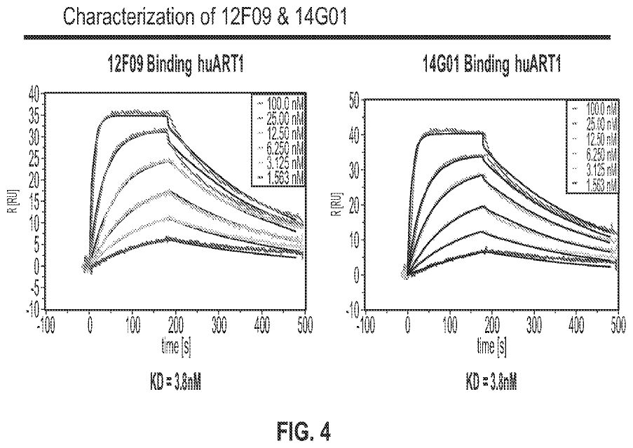

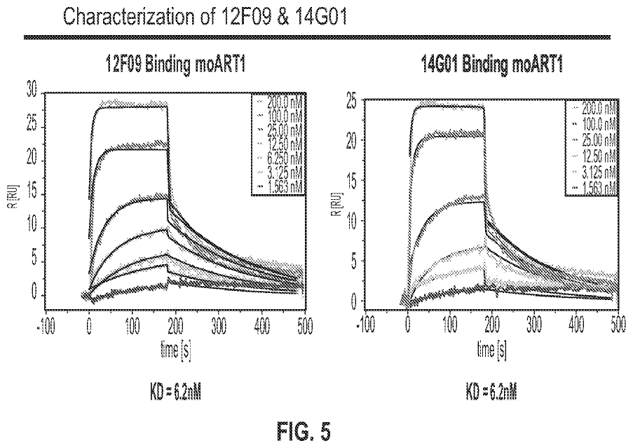

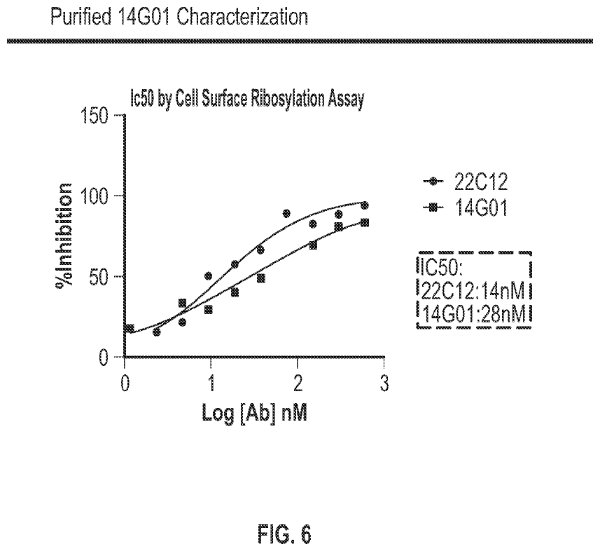

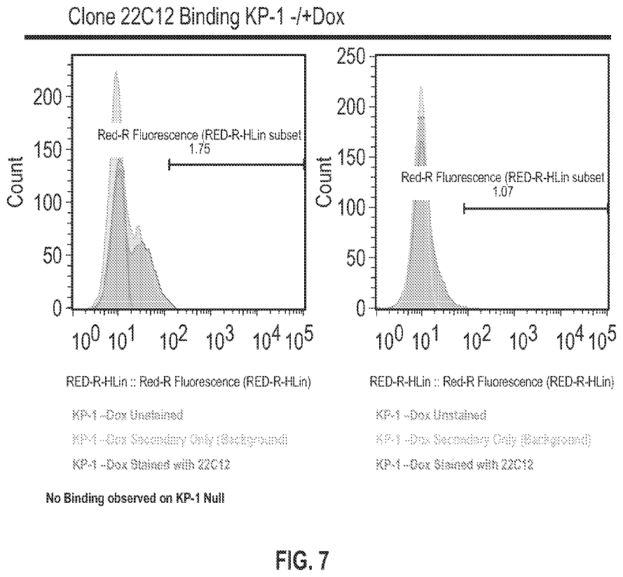

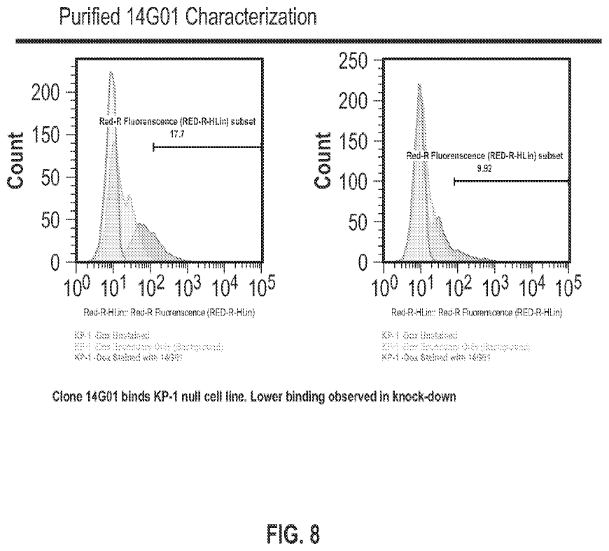

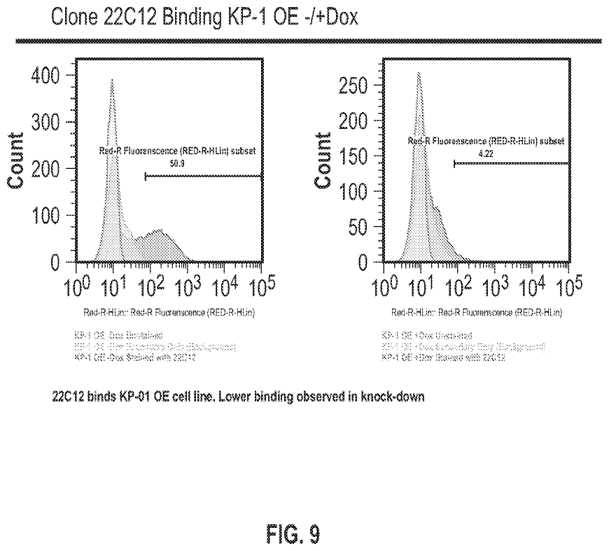

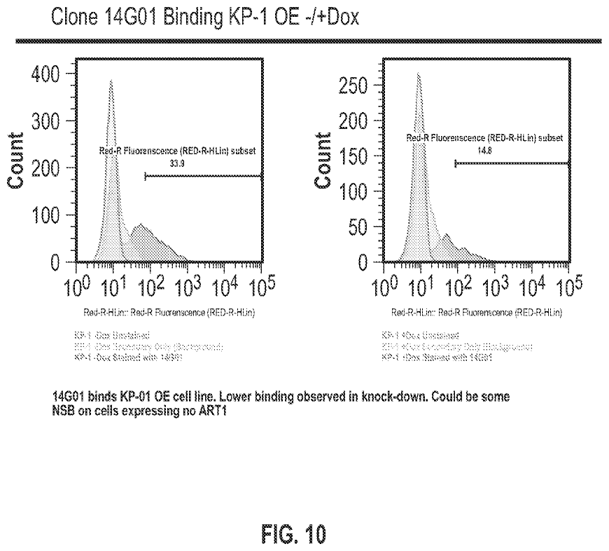

### DETAILED DESCRIPTION

Immune checkpoint inhibitors (ICI), alone or in combination with chemotherapy, have become the standard of care in patients with advanced non-small cell lung cancer (NSCLC) without targetable molecular alterations (Mok et al., 2019). However, the majority of lung cancer patients either do not respond to or do not experience long-term benefit from ICI, including many of those patients with high tumor PD-L1 expression (Gandhi et al., 2018; Gandini et al., 2016). Thus, there is an urgent need to identify other robust biomarkers predictive of response to ICI, and to understand the mechanisms of primary and acquired resistance of lung cancer to immunotherapy.

Cell surface mono-ADP ribosyltransferases (ARTs or ADPs) transfer the ADP-ribose moiety from NAD⁺ to amino acid residues to post-translationally modify target proteins. In humans, ADP-ribosyltransferase-1 (ART1) is expressed at low levels in healthy tissues including the lung. ART1 is a GPI-anchored enzyme, with an extracellular catalytic domain. Therefore ART1 may mono-ADP-ribosylate extracellular proteins in the local microenvironment, altering their function (Stevens et al., 2009; Okazaki et al., 1994; Balducci et al., 1999). The expression of ART1 in lung cancer has not been investigated, but previous studies have suggested increased ART1 protein expression in colorectal cancer and in glioblastoma, where high expression was associated with a poor prognosis (Tang et al., 2013). In mouse models of colorectal cancer, ART1 expression was shown to promote a more aggressive phenotype with increased epithelial-to-mesenchymal transition and increased angiogenesis (Yang et al., 2016; Song et al., 2016). However, it has not been determined whether tumor ART1 expression could regulate tumor cross-talk with the immune microenvironment.

Among the targets of ADP-ribosyl transferases is the P2X7 receptor (P2X7R, gene id: P2rx7). P2X7R is an ATP-gated cation channel of the purinergic type 2 receptor family, with low affinity for extracellular ATP, that activates pro-inflammatory pathways (Burnstock & Knight, 2004). It is expressed on multiple immune cell subsets including T cells and its expression is essential for inflammatory responses and anti-tumor immunity (Adinolfi et al., 2015; Haag et al., 2007). In NSCLC, high P2X7R expression has been associated with improved overall and progression-free survival (Boldrini et al., 2015). In pathological conditions such as tissue damage, tumor development, or inflammation, cytosolic NAD⁺ is released into the local extracellular environment where it may be used as a substrate by extracellular ADP-ribosyl transferases to catalyze the transfer of the ADP-ribose to P2X7R (Haag et al., 2007). This covalent modification results in constitutive activation of P2X7R leading to large pore formation, uncontrolled calcium influx, phosphatidylserine externalization, and ultimately a process described as NAD-induced cell death (NICD) (Scheuplein et al., 2009). Typically, extracellular NAD⁺ concentrations are generally low and tightly regulated by the ADP-ribosyl cyclase CD38, which is expressed on activated immune cells as well as on cancer cells (Sandoval-Montes & Santos-Argumedo, 2005; Chen et al., 2018). However, even in the presence of CD38, extracellular NAD⁺ concentrations can increase following rapid release from stressed or dying cells (Haag et al., 2007). In preclinical studies, ART-mediated NICD of T cells has been proposed as a homeostatic mechanism to eliminate naïve and bystander T cells in inflamed tissues (Adriouch et al., 2007). More recently, NICD was shown to regulate the homeostasis of tissue-resident memory T cells (TRMs), the presence of which in lung tumors has been associated with good prognosis (Stark et al., 2018; Nizard et al., 2017).

Escape from immune-mediated rejection enables tumor progression in non-small cell lung cancer (NSCLC) and can be countered in a subset of patients by therapeutic immune checkpoint inhibition (ICI) which restores anti-tumor immune functions. However, the majority of NSCLC patients do not respond to ICI, suggesting the existence of additional mechanisms of tumor immune escape. In inflamed tissues, where concentrations of extracellular NAD⁺ are high, NAD-induced cell death (NICD) of P2X7-receptor (P2X7R)-expressing T cells mediated by mono-ADP-ribosyltransferases (ARTs) regulates immune homeostasis.

Epithelial cells in the injured or inflamed lung may overexpress ART1 as a mechanism of cell survival to protect against cell clearance by inflammatory cells. An evolutionarily conserved parallel protective role was hypothesized to be provided by ART1 expression in lung cancer cells. An analogy may be drawn between ART1 and immune checkpoint pathways. In both cases, evolutionary mechanisms that exist to protect tissues from collateral damage at sites of inflammation are utilized by cancers to evade the immune response. Given the success of checkpoint inhibition as a strategy to overcome immune escape and effectively treat metastatic lung cancer, a similar strategy was envisioned for ART1 inhibition.

ART1 is overexpressed in lung cancers, is cytoprotective, and facilitates metastatic growth. As described herein, inhibitors of mono-ADP-ribosylation were identified to utilize for therapeutic inhibition of cancers. For example, using biobanked human materials, evidence of ART1 expression in human NSCLC tumors was found using whole tumor RT-PCR, immunofluorescence, and immunohistochemistry. Compared to matched adjacent normal lung (n=40), by RT-PCR there is over a 2-fold increase (p=0.01) in median tumor expression of ART1, suggesting a role in tumorigenesis or tumor progression. Heterogeneous expression existed by RT-PCR, implying that ART1 tumor expression may be more apparent in distinct subgroups of patients. Subsequently a tissue microarray containing 184 cases of predominantly (74%) stage I NSCLC was stained to determine the prevalence of NSCLC tumors staining positive for ART1. ART1 staining was moderate or strong in 83% of adenocarcinomas (n=145) and in 45% of squamous cell cancers (n=39, p<0.001). ART1 expression was found in all stage IV tumors.

In order to determine whether ART1 expression contributes to distinct phenotypic characteristics in lung cancer, ART1 was knocked down in a KRASG12D/+/p53^{-/-} cell line, KP1 (developed from a genetically engineered mouse model), using shRNA technology (sh175KP1). In a tail vein injection model in immunocompetent mice, a highly significant decrease in metastasis was noted in the ART1-knockdown cell lines compared to their parent lines. An in vitro model was employed to assess the ability of freshly procured neutrophils from immunocompetent mice to induce apoptosis in lung cancer cells. Strikingly, at a neutrophil: tumor cell ratio of 20:1, the knockdown cell line sh175KP1 lacking ART1 expression is more sensitive to neutrophil-induced apoptosis in the co-culture assay (87% vs. 56% Annexin V positive, p=0.05). This is consistent with the protective effect of ART1 expression on alveolar epithelial cells against neutrophil-derived proteins. Chemical inhibition of mono-ADP-ribosylation in the parent KP1 cell line with two well established inhibitors facilitated neutrophil-induced apoptosis, implying that the enzymatic activity of ART1 is critical to the phenotype. Based on this, it was hypothesized that ART1 expression is cytoprotective to lung cancer cells and facilitates metastatic outgrowth of circulating cells through its inhibitory actions on tumor suppressive immune cells or soluble proteins in the blood or metastatic niche. It is likely that mono-ADP ribosylation also affects other immune cells in the tumor microenvironment, particularly T cells. Because ART1 is an extracellular enzymatic target, it is highly druggable and thus susceptible to therapeutic intervention.

### Definitions

A “vector” refers to a macromolecule or association of macromolecules that comprises or associates with a polynucleotide, and which can be used to mediate delivery of the polynucleotide to a cell, either in vitro or in vivo. Illustrative vectors include, for example, plasmids, viral vectors, liposomes and other gene delivery vehicles. The polynucleotide to be delivered, sometimes referred to as a “target polynucleotide” or “transgene,” may comprise a coding sequence of interest in gene therapy (such as a gene encoding a protein of therapeutic interest), a coding sequence of interest in vaccine development (such as a polynucleotide expressing a protein, polypeptide or peptide suitable for eliciting an immune response in a mammal), and/or a selectable or detectable marker.

“Transduction,” “transfection,” “transformation” or “transducing” as used herein, are terms referring to a process for the introduction of an exogenous polynucleotide into a host cell leading to expression of the polynucleotide, e.g., the transgene in the cell, and includes the use of recombinant virus to introduce the exogenous polynucleotide to the host cell. Transduction, transfection or transformation of a polynucleotide in a cell may be determined by methods well known to the art including, but not limited to, protein expression (including steady state levels), e.g., by ELISA, flow cytometry and Western blot, measurement of DNA and RNA by heterologousization assays, e.g., Northern blots, Southern blots and gel shift mobility assays. Methods used for the introduction of the exogenous polynucleotide include well-known techniques such as viral infection or transfection, lipofection, transformation and electroporation, as well as other non-viral gene delivery techniques. The introduced polynucleotide may be stably or transiently maintained in the host cell.

“Gene delivery” refers to the introduction of an exogenous polynucleotide into a cell for gene transfer, and may encompass targeting, binding, uptake, transport, localization, replicon integration and expression.

“Gene transfer” refers to the introduction of an exogenous polynucleotide into a cell which may encompass targeting, binding, uptake, transport, localization and replicon integration, but is distinct from and does not imply subsequent expression of the gene.

“Gene expression” or “expression” refers to the process of gene transcription, translation, and post-translational modification.

The term “polynucleotide” refers to a polymeric form of nucleotides of any length, including deoxyribonucleotides or ribonucleotides, or analogs thereof. A polynucleotide may comprise modified nucleotides, such as methylated or capped nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the disclosure described herein that is a polynucleotide encompasses both the double-stranded form and each of two complementary single-stranded forms known or predicted to make up the double-stranded form.

“Nucleic acid sequence” is intended to encompass a polymer of DNA or RNA, i.e., a polynucleotide, which can be single-stranded or double-stranded and which can contain non-natural or altered nucleotides. The terms “nucleic acid” and “polynucleotide” as used herein refer to a polymeric form of nucleotides of any length, either ribonucleotides (RNA) or deoxyribonucleotides (DNA). These terms refer to the primary structure of the molecule, and thus include double- and single-stranded DNA, and double- and single-stranded RNA. The terms include, as equivalents, analogs of either RNA or DNA made from nucleotide analogs and modified polynucleotides such as, though not limited to, methylated and/or capped polynucleotides.

An “isolated” polynucleotide, e.g., plasmid, virus, polypeptide or other substance refers to a preparation of the substance devoid of at least some of the other components that may also be present where the substance or a similar substance naturally occurs or is initially prepared from. Thus, for example, an isolated substance may be prepared by using a purification technique to enrich it from a source mixture. Isolated nucleic acid, peptide or polypeptide is present in a form or setting that is different from that in which it is found in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. The isolated nucleic acid molecule may be present in single-stranded or double-stranded form. When an isolated nucleic acid molecule is to be utilized to express a protein, the molecule will contain at a minimum the sense or coding strand (i.e., the molecule may single-stranded), but may contain both the sense and anti-sense strands (i.e., the molecule may be double-stranded). Enrichment can be measured on an absolute basis, such as weight per volume of solution, or it can be measured in relation to a second, potentially interfering substance present in the source mixture. Increasing enrichments of the embodiments of this disclosure are envisioned. Thus, for example, a 2-fold enrichment, 10-fold enrichment, 100-fold enrichment, or a 1000-fold enrichment.

A “transcriptional regulatory sequence” (TRS) refers to a genomic region that controls the transcription of a gene or coding sequence to which it is operably linked. Transcriptional regulatory sequences of use in the present disclosure generally include at least one transcriptional promoter and may also include one or more enhancers and/or terminators of transcription.

“Operably linked” refers to an arrangement of two or more components, wherein the components so described are in a relationship permitting them to function in a coordinated manner. By way of illustration, a transcriptional regulatory sequence or a promoter is operably linked to a coding sequence if the TRS or promoter promotes transcription of the coding sequence. An operably linked TRS is generally joined in cis with the coding sequence, but it is not necessarily directly adjacent to it.

“Heterologous” means derived from a genotypically distinct entity from the entity to which it is compared. For example, a polynucleotide introduced by genetic engineering techniques into a different cell type is a heterologous polynucleotide (and, when expressed, can encode a heterologous polypeptide). Similarly, a transcriptional regulatory element such as a promoter that is removed from its native coding sequence and operably linked to a different coding sequence is a heterologous transcriptional regulatory element.

A “terminator” refers to a polynucleotide sequence that tends to diminish or prevent read-through transcription (i.e., it diminishes or prevent transcription originating on one side of the terminator from continuing through to the other side of the terminator). The degree to which transcription is disrupted is typically a function of the base sequence and/or the length of the terminator sequence. In particular, as is well known in numerous molecular biological systems, particular DNA sequences, generally referred to as “transcriptional termination sequences” are specific sequences that tend to disrupt read-through transcription by RNA polymerase, presumably by causing the RNA polymerase molecule to stop and/or disengage from the DNA being transcribed. Typical example of such sequence-specific terminators include polyadenylation (“polyA”) sequences, e.g., SV40 polyA. In addition to or in place of such sequence-specific terminators, insertions of relatively long DNA sequences between a promoter and a coding region also tend to disrupt transcription of the coding region, generally in proportion to the length of the intervening sequence. This effect presumably arises because there is always some tendency for an RNA polymerase molecule to become disengaged from the DNA being transcribed, and increasing the length of the sequence to be traversed before reaching the coding region would generally increase the likelihood that disengagement would occur before transcription of the coding region was completed or possibly even initiated. Terminators may thus prevent transcription from only one direction (“uni-directional” terminators) or from both directions (“bi-directional” terminators), and may be comprised of sequence-specific termination sequences or sequence-non-specific terminators or both. A variety of such terminator sequences are known in the art; and illustrative uses of such sequences within the context of the present disclosure are provided below.

“Host cells,” “cell lines,” “cell cultures,” “packaging cell line” and other such terms denote higher eukaryotic cells, such as mammalian cells including human cells, useful in the present disclosure, e.g., to produce recombinant virus or recombinant fusion polypeptide. These cells include the progeny of the original cell that was transduced. It is understood that the progeny of a single cell may not necessarily be completely identical (in morphology or in genomic complement) to the original parent cell.

“Recombinant,” as applied to a polynucleotide means that the polynucleotide is the product of various combinations of cloning, restriction and/or ligation steps, and other procedures that result in a construct that is distinct from a polynucleotide found in nature. A recombinant virus is a viral particle comprising a recombinant polynucleotide. The terms respectively include replicates of the original polynucleotide construct and progeny of the original virus construct.

A “control element” or “control sequence” is a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter. Promoters include AAV promoters, e.g., P5, P19, P40 and AAV ITR promoters, as well as heterologous promoters.

An “expression vector” is a vector comprising a region which encodes a gene product of interest, and is used for effecting the expression of the gene product in an intended target cell. An expression vector also comprises control elements operatively linked to the encoding region to facilitate expression of the protein in the target. The combination of control elements and a gene or genes to which they are operably linked for expression is sometimes referred to as an “expression cassette,” a large number of which are known and available in the art or can be readily constructed from components that are available in the art.

The terms “polypeptide” and “protein” are used interchangeably herein to refer to polymers of amino acids of any length. The terms also encompass an amino acid polymer that has been modified; for example, disulfide bond formation, glycosylation, acetylation, phosphonylation, lipidation, or conjugation with a labeling component.

The term “exogenous,” when used in relation to a protein, gene, nucleic acid, or polynucleotide in a cell or organism refers to a protein, gene, nucleic acid, or polynucleotide which has been introduced into the cell or organism by artificial or natural means. An exogenous nucleic acid may be from a different organism or cell, or it may be one or more additional copies of a nucleic acid which occurs naturally within the organism or cell. By way of a non-limiting example, an exogenous nucleic acid is in a chromosomal location different from that of natural cells, or is otherwise flanked by a different nucleic acid sequence than that found in nature, e.g., an expression cassette which links a promoter from one gene to an open reading frame for a gene product from a different gene.

“Transformed” or “transgenic” is used herein to include any host cell or cell line, which has been altered or augmented by the presence of at least one recombinant DNA sequence. The host cells of the present disclosure are typically produced by transfection with a DNA sequence in a plasmid expression vector, as an isolated linear DNA sequence, or infection with a recombinant viral vector.

The term “sequence homology” means the proportion of base matches between two nucleic acid sequences or the proportion amino acid matches between two amino acid sequences. When sequence homology is expressed as a percentage, e.g., 50%, the percentage denotes the proportion of matches over the length of a selected sequence that is compared to some other sequence. Gaps (in either of the two sequences) are permitted to maximize matching; gap lengths of 15 bases or less are usually used, 6 bases or less are preferred with 2 bases or less more preferred. When using oligonucleotides as probes or treatments, the sequence homology between the target nucleic acid and the oligonucleotide sequence is generally not less than 17 target base matches out of 20 possible oligonucleotide base pair matches (85%); not less than 9 matches out of 10 possible base pair matches (90%), or not less than 19 matches out of 20 possible base pair matches (95%).

Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. The two sequences or parts thereof are more homologous if their amino acids are greater than or equal to 50% identical when optimally aligned using the ALIGN program.

The term “corresponds to” is used herein to mean that a polynucleotide sequence is structurally related to all or a portion of a reference polynucleotide sequence, or that a polypeptide sequence is structurally related to all or a portion of a reference polypeptide sequence, e.g., they have at least 80%, 85%, 90%, 95% or more, e.g., 99% or 100%, sequence identity. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

The term “sequence identity” means that two polynucleotide sequences are identical (i.e., on a nucleotide-by-nucleotide basis) over the window of comparison. The term “percentage of sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denote a characteristic of a polynucleotide sequence, wherein the polynucleotide comprises a sequence that has at least 85 percent sequence identity, preferably at least 90 to 95 percent sequence identity, more usually at least 99 percent sequence identity as compared to a reference sequence over a comparison window of at least 20 nucleotide positions, frequently over a window of at least 20-50 nucleotides, wherein the percentage of sequence identity is calculated by comparing the reference sequence to the polynucleotide sequence which may include deletions or additions which total 20 percent or less of the reference sequence over the window of comparison.

“Conservative” amino acid substitutions are, for example, aspartic-glutamic as polar acidic amino acids; lysine/arginine/histidine as polar basic amino acids; leucine/isoleucine/methionine/valine/alanine/glycine/proline as non-polar or hydrophobic amino acids; serine/threonine as polar or uncharged hydrophilic amino acids. Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting polypeptide. Whether an amino acid change results in a functional polypeptide can readily be determined by assaying the specific activity of the polypeptide. Naturally occurring residues are divided into groups based on common side-chain properties: (1) hydrophobic: norleucine, met, ala, val, leu, ile; (2) neutral hydrophilic: cys, ser, thr; (3) acidic: asp, glu; (4) basic: asn, gln, his, lys, arg; (5) residues that influence chain orientation: gly, pro; and (6) aromatic; trp, tyr, phe.

The disclosure also envisions polypeptides with non-conservative substitutions. Non-conservative substitutions entail exchanging a member of one of the classes described above for another.

By “ART1” is meant ADP ribosyltransferase 1, e.g., a mammalian ART1 having a sequence in Accession Nos. NP_004305.2, XP_0115184161, NP_033840.2, or XP_011239959.1, the disclosures of which are incorporated by reference herein, or a protein with at least 80%, 85%, 90%, 92%, 94%, 95%, 97%, 98% or 99% amino acid sequence identity thereto, alternatively spliced isoforms thereof, peptide fragments thereof or post-translationally modified proteins or peptides thereof. For example, an antibody within the scope of this disclosure may bind human ART1, e.g.,

(SEQ ID NO: 16)
MQMPAMMSLL LVSVGLMEAL QAQSHPITRR DLFSQEIQLD
MALASFDDQY AGCAAAMTAA LPDLNHTEFQ ANQVYADSWT
LASSQWQERQ ARWPEWSLSP TRPSPPPLGF RDEHGVALLA
YTANSPLHKE FNAAVREAGR SRAHYLHHFS FKTLHFLLTE
ALQLLGSGQR PPRCHQVFRG VHGLRFRPAG PRATVRLGGF
ASASLKHVAA QQFGEDTFFG IWTCLGAPIK GYSFFPGEEE
VLIPPFETFQ VINASRLAQG PARIYLRALG KHSTYNCEYI
KDKKCKSGPC HLDNSAMGQS PLSAVWSLLL LLWFLVVRAF
PDGPGLL
and/or
may bind mouse ART1, e.g.,
(SEQ ID NO: 88)
MKIPAMMSLL LVSVGLRDGV QVQSYSISQL DIFSQETPLD
MAPASFDDQY AGCLADMTAA LPDLNHSEFQ ANKVYADGWA
QANNQWQERR AWGSVWGSLP PSPPGFRDEH GVALLAYTAN
SPLHKEFNAA VREAGRSRAH YLHHFSFKTL HFLLTEALQL
LRSHRSRGCQ QVYRGVHGLR FRPAGPGATV RLGGFASASL
KNVAAQQFGE DTFFGIWTCL GAPIRGYSFF PEEEEVLIPP
FETFQVINTS RPTQGPARIY LRALGKRSTY NCEYIKEKKC
RSGPCWLGSS APGSISASCS LLLLLLFLVL SALPENPGLQ
QLTRC.

The term “antibody,” as used herein, refers to a full-length immunoglobulin molecule or an immunologically-active fragment of an immunoglobulin molecule such as the Fab or F(ab′)2 fragment generated by, for example, cleavage of the antibody with an enzyme such as pepsin or co-expression of an antibody light chain and an antibody heavy chain in, for example, a mammalian cell, or ScFv. The antibody can also be an IgG, IgD, IgA, IgE or IgM antibody. Full-length immunoglobulin “light chains” (about 25 kD or 214 amino acids) are encoded by a variable region gene at the amino-terminus (about 110 amino acids) and a kappa or lambda constant region gene at the carboxy-terminus. Full-length immunoglobulin “heavy chains” (about 50 kD or 446 amino acids), are similarly encoded by a variable region gene (about 116 amino acids) and one of the other aforementioned constant region genes, e.g., gamma (encoding about 330 amino acids). Heavy chains are classified as gamma, mu, alpha, delta, or epsilon, which in turn define the immunoglobulin classes, IgG, IgM, IgA, IgD and IgE, respectively. An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively. In each pair of the tetramer, the light and heavy chain variable regions are together responsible for binding to an antigen, and the constant regions are responsible for the antibody effector functions. In addition to naturally occurring antibodies, immunoglobulins may exist in a variety of other forms including, for example, Fv, ScFv, Fab, and F(ab′) 2, as well as bifunctional hybrid antibodies (e.g., Lanzavecchia et al. (1987)) and in single chains (e.g., Huston et al. (1988) and Bird et al. (1988), which are incorporated herein by reference). (See, generally, Hood et al., “Immunology”, Benjamin, N.Y., 2^{nd} ed. (1984), and Hunkapiller and Hood (1986), which are incorporated herein by reference). Thus, the term “antibody” includes antigen binding antibody fragments, as are known in the art, including Fab, Fab₂, single chain antibodies (scFv for example), chimeric antibodies, etc., either produced by the modification of whole antibodies or those synthesized de novo using recombinant DNA technologies.

An immunoglobulin light or heavy chain variable region consists of a “framework” region interrupted by three hypervariable regions, also called CDR's. The extent of the framework region and CDR's have been precisely defined (see, “Sequences of Proteins of Immunological Interest,” E. Kabat et al., U.S. Department of Health and Human Services, (1983); which is incorporated herein by reference). The sequences of the framework regions of different light or heavy chains are relatively conserved within a species. As used herein, a “human framework region” is a framework region that is substantially identical (about 85% or more, usually 90 to 95% or more) to the framework region of a naturally occurring human immunoglobulin. The framework region of an antibody, that is the combined framework regions of the constituent light and heavy chains, serves to position and align the CDR's. The CDR's are primarily responsible for binding to an epitope of an antigen.

Chimeric antibodies are antibodies whose light and heavy chain genes have been constructed, typically by genetic engineering, from immunoglobulin variable and constant region genes belonging to different species. For example, the variable segments of the genes from a mouse monoclonal antibody may be joined to human constant segments, such as gamma 1 and gamma 3. One example of a chimeric antibody is one composed of the variable or antigen-binding domain from a mouse antibody and the constant or effector domain from a human antibody, although other mammalian species may be used.

As used herein, the term “humanized” immunoglobulin refers to an immunoglobulin having a human framework region and one or more CDR's from a non-human (usually a mouse or rat) immunoglobulin. The non-human immunoglobulin providing the CDR's is called the “donor” and the human immunoglobulin providing the framework is called the “acceptor.” Constant regions need not be present, but if they are, they are generally substantially identical to human immunoglobulin constant regions, i.e., at least about 85-90%, or about 95% or more identical. Hence, all parts of a humanized immunoglobulin, except possibly the CDR's, are substantially identical to corresponding parts of natural human immunoglobulin sequences. A “humanized antibody” is an antibody comprising a humanized light chain and a humanized heavy chain immunoglobulin. One says that the donor antibody has been “humanized”, by the process of “humanization”, because the resultant humanized antibody is expected to bind to the same antigen as the donor antibody that provides the CDR's.

Thus, humanized forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody has substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will include at least a portion of an immunoglobulin constant region (Fc), typically that of a human immunoglobulin (Jones et al. (1986); Riechmann et al. (1988); and Presta (1992)).

It is understood that the humanized antibodies may have additional conservative amino acid substitutions which have substantially no effect on antigen binding or other immunoglobulin functions. By conservative substitutions are intended combinations such as gly, ala; val, ile, leu; asp, glu; asn, gln; ser, thr; lys, arg; and phe, tyr.

Humanized immunoglobulins, including humanized antibodies, have been constructed by means of genetic engineering. Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522 (1986); Riechmann et al., Nature, 332:323 (1988); Verhoeyen et al., Science, 239:1534 (1988)), by substituting rodent CDRs or CDR sequences for the corresponding sequences of a human antibody. Accordingly, such “humanized” antibodies are chimeric antibodies that have substantially less than an intact human variable domain has been substituted by the corresponding sequence from a non-human species. In practice, humanized antibodies are typically human antibodies in which some CDR residues and possibly some framework residues are substituted by residues from analogous sites in rodent antibodies.

Human antibodies can also be produced using various techniques known in the art, including phage display libraries (Hoogenboom and Winter, J. Mol. Biol., 227:381 (1991); Marks et al., J. Mol. Biol., 222:581 (1991)). The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147:86 (1991)). Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10:779 (1992); Lonberg et al., Nature, 368:856 (1994); Morrison, Nature, 368:812 (1994); Fishwild et al., Nature Biotechnology, 14:845 (1996); Neuberger, Nature Biotechnology, 14:826 (1996); Lonberg and Huszar, Intern. Rev. Immunol., 13:65 (1995). Most humanized immunoglobulins that have been previously described have a framework that is identical to the framework of a particular human immunoglobulin chain and three CDR's from a non-human donor immunoglobulin chain.

A framework may be one from a particular human immunoglobulin that is unusually homologous to the donor immunoglobulin to be humanized, or a consensus framework derived from many human antibodies. For example, comparison of the sequence of a mouse heavy (or light) chain variable region against human heavy (or light) variable regions in a data bank (for example, the National Biomedical Research Foundation Protein Identification Resource) shows that the extent of homology to different human regions varies greatly, typically from about 40% to about 60-70%. By choosing one of the human heavy (respectively light) chain variable regions that is most homologous to the heavy (respectively light) chain variable region of the other immunoglobulin, fewer amino acids will be changed in going from the one immunoglobulin to the humanized immunoglobulin. The precise overall shape of a humanized antibody having the humanized immunoglobulin chain may more closely resemble the shape of the donor antibody, also reducing the chance of distorting the CDR's.

Typically, one of the 3-5 most homologous heavy chain variable region sequences in a representative collection of at least about 10 to 20 distinct human heavy chains is chosen as acceptor to provide the heavy chain framework, and similarly for the light chain. One of the 1 to 3 most homologous variable regions may be used. The selected acceptor immunoglobulin chain may have at least about 65% homology in the framework region to the donor immunoglobulin.

In many cases, it may be considered desirable to use light and heavy chains from the same human antibody as acceptor sequences, to be sure the humanized light and heavy chains will make favorable contacts with each other. Regardless of how the acceptor immunoglobulin is chosen, higher affinity may be achieved by selecting a small number of amino acids in the framework of the humanized immunoglobulin chain to be the same as the amino acids at those positions in the donor rather than in the acceptor.

Humanized antibodies generally have advantages over mouse or in some cases chimeric antibodies for use in human therapy: because the effector portion is human, it may interact better with the other parts of the human immune system (e.g., destroy the target cells more efficiently by complement-dependent cytotoxicity (CDC) or antibody-dependent cellular cytotoxicity (ADCC)); the human immune system should not recognize the framework or constant region of the humanized antibody as foreign, and therefore the antibody response against such an antibody should be less than against a totally foreign mouse antibody or a partially foreign chimeric antibody.

DNA segments having immunoglobulin sequences typically further include an expression control DNA sequence operably linked to the humanized immunoglobulin coding sequences, including naturally-associated or heterologous promoter regions. Generally, the expression control sequences will be eukaryotic promoter systems in vectors capable of transforming or transfecting eukaryotic host cells, but control sequences for prokaryotic hosts may also be used. Once the vector has been incorporated into the appropriate host, the host is maintained under conditions suitable for high level expression of the nucleotide sequences, and, as desired, the collection and purification of the humanized light chains, heavy chains, light/heavy chain dimers or intact antibodies, binding fragments or other immunoglobulin forms may follow (see, S. Beychok, Cells of Immunoglobulin Synthesis, Academic Press, New York, (1979), which is incorporated herein by reference).

Other “substantially homologous” modified immunoglobulins to the native sequences can be readily designed and manufactured utilizing various recombinant DNA techniques well known to those skilled in the art. For example, the framework regions can vary at the primary structure level by several amino acid substitutions, terminal and intermediate additions and deletions, and the like. Moreover, a variety of different human framework regions may be used singly or in combination as a basis for the humanized immunoglobulins of the present disclosure. In general, modifications of the genes may be readily accomplished by a variety of well-known techniques, such as site-directed mutagenesis (see, Gillman and Smith, Gene, 8:81 (1979) and Roberts et al., Nature, 328:731 (1987), both of which are incorporated herein by reference). Substantially homologous immunoglobulin sequences are those which exhibit at least about 85% homology, usually at least about 90%, or at least about 95% homology with a reference immunoglobulin protein.

Alternatively, polypeptide fragments comprising only a portion of the primary antibody structure may be produced, which fragments possess one or more immunoglobulin activities (e.g., antigen binding). These polypeptide fragments may be produced by proteolytic cleavage of intact antibodies by methods well known in the art, or by inserting stop codons at the desired locations in vectors known to those skilled in the art, using site-directed mutagenesis.

### Exemplary Anti-ART1 Molecules

The disclosure provides for an antibody, antigen binding fragment thereof, or a polypeptide, directed against ART1. In one embodiment, the antibody, fragment thereof, or polypeptide binds both human and mouse ART1, and so are likely bind to conserved sequences in those proteins (see the alignment below), while antibodies that bind human but not mouse ART1 likely bind to non-conserved sequences (see the alignment below).

MQMPAMMSLLLVSVGLMEALQAQSHPITRRDLFSQEIQLDMALASFDDQYAGCAAAMTAA
M++PAMMSLLLVSVGL + +Q QS+ I++ D+FSQE LDMA ASFDDQYAGC A MTAA
MKIPAMMSLLLVSVGLRDGVQVQSYSISQLDIFSQETPLDMAPASFDDQYAGCLADMTAA
LPDLNHTEFQANQVYADSWTLASSQWQERQARWPEWSLSPTRPSPPPLGFRDEHGVALLA
LPDLNH+EFQAN+VYAD W A++QWQER+A W P PSPP GFRDEHGVALLA
LPDLNHSEFQANKVYADGWAQANNQWQERRAWGSVWGSLP--PSPP--GFRDEHGVALLA
YTANSPLHKEFNAAVREAGRSRAHYLHHFSFKTLHFLLTEALQLLGSGQRPPRCHQVFRG
YTANSPLHKEFNAAVREAGRSRAHYLHHFSFKTLHFLLTEALQLL S R C QV+RG
YTANSPLHKEFNAAVREAGRSRAHYLHHFSFKTLHFLLTEALQLLRS-HRSRGCQQVYRG
VHGLRFRPAGPRATVRLGGFASASLKHVAAQQFGEDTFFGIWTCLGAPIKGYSFFPGEEE
VHGLRFRPAGP ATVRLGGFASASLK+VAAQQFGEDTFFGIWTCLGAPI+GYSFFP EEE
VHGLRFRPAGPGATVRLGGFASASLKNVAAQQFGEDTFFGIWTCLGAPIRGYSFFPEEEE
(SEQ ID NO: 90)
VLIPPFETFQVINASRLAQGPARIYLRALGKHSTYNCEYIKDKKCKSGPCHLDNSAMG
(SEQ ID NO: 91)
VLIPPFETFQVIN SR QGPARIYLRALGK STYNCEYIK+KKC+SGPC L +SAG
(SEQ ID NO: 92)
VLIPPFETFQVINTSRPTQGPARIYLRALGKRSTYNCEYIKEKKCRSGPCWLGSSAPG

Thus, in one embodiment, an antibody, antigen binding fragment thereof, or a polypeptide, directed against ART1 that binds to both human and mouse ART1 may bind to residues including those from position 170 to 185 (human numbering), 195 to 210, or 230 to 250, or a combination thereof. In one embodiment, antibodies that bind to ART1 include those that bind to residues including those from positon 110 to 160, 185 to 225 or 245 to 275, or a combination thereof, in ART1. In one embodiment, antibodies that bind to ART1 include those that bind to residues including those from positon 20 to 50, 80 to 100, 170 to 85, 225 to 245 or 275 to the C-terminus, or a combination thereof. In one embodiment, an antibody, antigen binding fragment thereof, or a polypeptide, binds to at least one of the following residues: S75, S77, T79, R80, R89, H92, or Y99 of human ART1, e.g., SEQ ID NO:90. In one embodiment, an isolated antibody or fragment thereof binds to two, three, four, five or six of S75, S77, T79, R80, R89, H92, or Y99 of human ART1, e.g., SEQ ID NO: 90. In one embodiment, an isolated antibody or fragment thereof binds to S75, S77, T79, R80, R89, H92, and Y99 of human ART1, e.g., SEQ ID NO:90.

One of ordinary skill in the art will appreciate that an antibody consists of four polypeptides: two identical copies of a heavy (H) chain polypeptide and two copies of a light (L) chain polypeptide. Each of the heavy chains contains one N-terminal variable (V_{H}) region and three C-terminal constant (CH1, CH2 and CH3) regions, and each light chain contains one N-terminal variable (V_{L}) region and one C-terminal constant (C_{L}) region. The variable regions of each pair of light and heavy chains form the antigen binding site of an antibody. The nucleic acid sequence which encodes an antibody directed against ART1 can comprise one or more nucleic acid sequences, each of which encodes one or more of the heavy and/or light chain polypeptides of an anti-ART1 antibody. In this respect, the nucleic acid sequence which encodes an antibody directed against ART1 can comprise a single nucleic acid sequence that encodes the heavy chain polypeptide and the light chain polypeptide of an anti-ART1 antibody. Alternatively, the nucleic acid sequence which encodes an antibody directed against ART1 can comprise a first nucleic acid sequence that encodes the heavy chain polypeptide of an anti-ART1 antibody, and a second nucleic acid sequence that encodes the light chain polypeptide of an anti-ART1 antibody. In yet another embodiment, the nucleic acid sequence which encodes a fragment of an antibody directed against ART1 can comprise a nucleic acid sequence encoding a heavy chain variable region polypeptide of an anti-ART1 antibody, a nucleic acid sequence encoding a light chain variable region polypeptide of an anti-ART1 antibody, or a nucleic acid sequence encoding a heavy chain variable region and a light chain variable region polypeptide of an anti-ART1 antibody.

In another embodiment, the nucleic acid sequence which encodes an antibody directed against ART1 encodes an antigen-binding fragment (also referred to as an “antibody fragment”) of an anti-ART1 antibody. The term “antigen-binding fragment” refers to one or more fragments of an antibody that retain the ability to specifically bind to an antigen (e.g., ART1) (see, generally, Holliger and Hudson 2005). Examples of antigen-binding fragments include but are not limited to (i) a Fab fragment, which is a monovalent fragment consisting of the V_{L}, V_{H}, C_{L}, and C_{H1} domains; (ii) a F(ab′)2 fragment, which is a bivalent fragment comprising two Fab fragments linked by a disulfide bridge at the hinge region; and (iii) a Fv fragment consisting of the V_{L} and V_{H} domains of a single arm of an antibody. In one embodiment, the nucleic acid sequence which encodes an antibody directed against ART1 can comprise a nucleic acid sequence encoding a Fab fragment of an anti-ART1 antibody. In one embodiment, the nucleic acid sequence which encodes an antibody or fragment thereof directed against ART1 can comprise a nucleic acid sequence encoding a heavy chain variable region that binds ART1. In one embodiment, the nucleic acid sequence which encodes an antibody or fragment thereof directed against ART1 can comprise a nucleic acid sequence encoding a light chain variable region that binds ART1. In one embodiment, the nucleic acid sequence which encodes an antibody directed against ART1 can comprise a nucleic acid sequence encoding one, two or three CDRs, e.g., of a heavy chain variable region, that bind(s) ART1. In one embodiment, the nucleic acid sequence which encodes an antibody directed against ART1 can comprise a nucleic acid sequence encoding one, two or three CDRs, e.g., of a light chain variable region, that bind(s) ART1. The antibody fragment may be a scFv antibody or a nanobody (VHH antibodies having a single variable domain in a heavy chain), Fab or F(ab′)₂.

In one embodiment, the nucleic acid sequence can encode the ART1-binding monoclonal antibody 22C12 or a fragment thereof. In one embodiment, the nucleic acid sequence can encode the ART1-binding monoclonal antibody 14G01 or a fragment thereof.

In an embodiment, the nucleic acid sequence which encodes an antibody against ART1 that recognizes (binds) human and mouse ART1. In an embodiment, the nucleic acid sequence which encodes an antibody against ART1 recognizes human but not mouse ART1.

An antibody, or antigen-binding fragment thereof, can be obtained by any means, including via in vitro sources (e.g., a hybridoma or a cell line producing an antibody recombinantly) and in vivo sources (e.g., rodents).

Methods for generating antibodies are known in the art and are described in, for example, Köhler and Milstein, Eur. J. Immunol., 5:511 (1976); Harlow and Lane (eds.), Antibodies: A Laboratory Manual, CSH Press (1988); and C. A. Janeway et al. (eds.), Immunobiology, 5th Ed., Garland Publishing, New York, NY (2001)). In certain embodiments, a human antibody or a chimeric antibody can be generated using a transgenic animal (e.g., a mouse) wherein one or more endogenous immunoglobulin genes are replaced with one or more human immunoglobulin genes. Examples of transgenic mice wherein endogenous antibody genes are effectively replaced with human antibody genes include, but are not limited to, the AlivaMab® mouse, VelocImmune mouse, Trianni® mouse, Kymab™ mouse, HUMAB-MOUSE™, the Kirin TC MOUSE™, and the KM-MOUSE™ (see, e.g., Lonberg, Nat. Biotechnol., 23 (9): 1117 (2005), and Lonberg, Handb. Exp. Pharmacol., 181:69 (2008)).

The nucleic acid sequence which encodes an antibody directed against ART1, an antigen-binding fragment thereof, or a polypeptide that binds ART1, can be generated using methods known in the art. For example, polypeptides, and proteins can be recombinantly produced using standard recombinant DNA methodology (see, e.g., Sambrook et al., Molecular Cloning: A Laboratory Manual, 3rd ed., Cold Spring Harbor Press, Cold Spring Harbor, NY, 2001; and Ausubel et al., Current Protocols in Molecular Biology, Greene Publishing Associates and John Wiley & Sons, NY, 1994). Further, an antibody directed against ART1, or an antigen-binding fragment thereof, can be isolated and/or purified from a source, such as a bacterium, an insect, or a mammal, e.g., a rat, a human, etc., into which a synthetically produced nucleic acid sequences encoding such antibody or antigen-binding fragment was introduced. Methods of isolation and purification are well-known in the art. Alternatively, the nucleic acid sequences described herein can be commercially synthesized. In this respect, the nucleic acid sequence can be synthetic, recombinant, isolated, and/or purified.

The nucleic acid sequence which encodes an antibody directed against ART1 may be identified by extracting RNA from the available antibody producing hybridoma cells. cDNA is produced by reverse transcription and PCR amplification of the light and heavy chains and is carried out using a rapid amplification of cDNA ends (RACE) strategy in combination with specific primers for conserved regions in the constant domains.

The nucleic acid sequence which encodes an antibody directed against ART1 may also be fully or partly humanized by means known in the art. For example, an antibody chimera may be created by substituting DNA encoding the mouse Fc region of the antibody with that of cDNA encoding for human.

The Fab portion of the molecule may also be humanized by selectively altering the DNA of non-CDR portions of the Fab sequence that differ from those in humans by exchanging the sequences for the appropriate individual amino acids.

Alternatively, humanization may be achieved by insertion of the appropriate CDR coding segments into a human antibody “scaffold”.

Resulting antibody DNA sequences may be modified for high expression levels in mammalian cells through removal of RNA instability elements and/or codon optimization, as is known in the art.

In an embodiment, nucleic acid sequences which encode the heavy chain and light chain of an antibody directed against ART1, may be expressed under the control of a single promoter in a 1:1 ratio using a 2A sequence (a cis-acting hydrolase element) self-cleavable sequence. The 2A sequence self-cleaves during protein translation and leaves a short tail of amino acids in the C-terminus of the upstream protein. A Furin cleavage recognition site may be added between the 2A sequence and the upstream gene to assure removal of the remaining amino acids. Plasmids expressing the correct inserts may be identified by DNA sequencing and by antibody specific binding using western analysis and ELISA assays.

### Exemplary Gene Transfer Vectors

The disclosure also provides a gene transfer vector comprising a nucleic acid sequence which encodes an antibody, an antigen binding fragment thereof, or a polypeptide, directed against ART1. In one embodiment, the gene transfer vector is a virus. The disclosure further provides a method of using the gene transfer vector or encoded gene product against ART1 in a mammal, which method comprises administering to the mammal the above-described gene transfer vector or the encoded gene product. Various aspects of the gene transfer vector, antibody or antigen binding fragment thereof, and methods are discussed below. Although each parameter is discussed separately, the gene transfer vector, antibody or antigen binding fragment thereof, or polypeptide, and method, may comprise combinations of the parameters set forth below. Accordingly, any combination of parameters can be used according to the gene transfer vector, antibody or antigen binding fragment thereof, the polypeptide, and the method.

A “gene transfer vector” is any molecule or composition that has the ability to carry and deliver a heterologous nucleic acid sequence into a suitable host cell where synthesis of the encoded protein takes place. Typically, a gene transfer vector is a nucleic acid molecule that has been engineered, using recombinant DNA techniques that are known in the art, to incorporate the heterologous nucleic acid sequence. Desirably, the gene transfer vector is comprised of DNA. Examples of suitable DNA-based gene transfer vectors include plasmids and viral vectors. However, gene transfer vectors that are not based on nucleic acids, such as liposomes, are also known and used in the art. The gene transfer vector can be based on a single type of nucleic acid (e.g., a plasmid) or non-nucleic acid molecule (e.g., a lipid or a polymer). The gene transfer vector can be integrated into the host cell genome, or can be present in the host cell in the form of an episome.

In one embodiment, the gene transfer vector is a viral vector. Suitable viral vectors include, for example, retroviral vectors, herpes simplex virus (HSV)-based vectors, parvovirus-based vectors, e.g., adeno-associated virus (AAV)-based vectors, AAV-adenoviral chimeric vectors, and adenovirus-based vectors. These viral vectors can be prepared using standard recombinant DNA techniques described in, for example, Sambrook et al., Molecular Cloning, a Laboratory Manual, 3rd edition, Cold Spring Harbor Press, Cold Spring Harbor, N.Y. (2001), and Ausubel et al., Current Protocols in Molecular Biology, Greene Publishing Associates and John Wiley & Sons, New York, N.Y. (1994).

Any viral vector may be employed to deliver antibody encoding sequences to cells including mammalian cells, or to mammals, include but are not limited to adeno-associated virus, adenovirus, herpesvirus, retrovirus, or lentivirus vectors.

In addition to the nucleic acid sequence encoding an antibody against ART1, or an antigen-binding fragment thereof, the viral vector may comprise expression control sequences, such as promoters, enhancers, polyadenylation signals, transcription terminators, internal ribosome entry sites (IRES), and the like, that provide for the expression of the nucleic acid sequence in a host cell. Exemplary expression control sequences are known in the art and described in, for example, Goeddel, Gene Expression Technology: Methods in Enzymology, Vol. 185, Academic Press, San Diego, CA. (1990).

A large number of promoters, including constitutive, inducible, and repressible promoters, from a variety of different sources are well known in the art. Representative sources of promoters include for example, virus, mammal, insect, plant, yeast, and bacteria, and suitable promoters from these sources are readily available, or can be made synthetically, based on sequences publicly available, for example, from depositories such as the ATCC as well as other commercial or individual sources. Promoters can be unidirectional (i.e., initiate transcription in one direction) or bi-directional (i.e., initiate transcription in either a 3′ or 5′ direction). Non-limiting examples of promoters include, for example, the T7 bacterial expression system, pBAD (araA) bacterial expression system, the cytomegalovirus (CMV) promoter, the SV40 promoter, and the RSV promoter. Inducible promoters include, for example, the Tet system (U.S. Pat. Nos. 5,464,758 and 5,814,618), the Ecdysone inducible system (No et al., Proc. Natl. Acad. Sci., 93:3346 (1996)), the T-REX™ system (Invitrogen, Carlsbad, CA), LACSWITCH™ System (Stratagene, San Diego, CA), and the Cre-ERT tamoxifen inducible recombinase system (Indra et al., Nuc. Acid. Res., 27:4324 (1999); Nuc. Acid. Res., 28: e99 (2000); U.S. Pat. No. 7,112,715; and Kramer & Fussenegger, Methods Mol. Biol., 308:123 (2005)).

The term “enhancer” as used herein, refers to a DNA sequence that increases transcription of, for example, a nucleic acid sequence to which it is operably linked. Enhancers can be located many kilobases away from the coding region of the nucleic acid sequence and can mediate the binding of regulatory factors, patterns of DNA methylation, or changes in DNA structure. A large number of enhancers from a variety of different sources are well known in the art and are available as or within cloned polynucleotides (from, e.g., depositories such as the ATCC as well as other commercial or individual sources). A number of polynucleotides comprising promoters (such as the commonly-used CMV promoter) also comprise enhancer sequences. Enhancers can be located upstream, within, or downstream of coding sequences. In one embodiment, the nucleic acid sequence encoding an antibody against ART1, or an antigen-binding fragment thereof, is operably linked to a CMV enhancer/chicken beta-actin promoter (also referred to as a “CAG promoter”) (see, e.g., Niwa et al., Gene, 108:193 (1991); Daly et al., Proc. Natl. Acad. Sci. U.S.A., 96:2296 (1999); and Sondhi et al., Mol. Ther., 15:481 (2007)).

Typically AAV vectors are produced using well characterized plasmids. For example, human embryonic kidney 293T cells are transfected with one of the transgene specific plasmids and another plasmid containing the adenovirus helper and AAV rep and cap genes (specific to AAVrh. 10, 8 or 9 as required). After 72 hours, the cells are harvested and the vector is released from the cells by five freeze/thaw cycles. Subsequent centrifugation and benzonase treatment removes cellular debris and unencapsidated DNA. lodixanol gradients and ion exchange columns may be used to further purify each AAV vector. Next, the purified vector is concentrated by a size exclusion centrifuge spin column to the required concentration. Finally, the buffer is exchanged to create the final vector products formulated (for example) in 1× phosphate buffered saline. The viral titers may be measured by TaqMan® real-time PCR and the viral purity may be assessed by SDS-PAGE.

### Exemplary Pharmaceutical Compositions and Delivery

The disclosure provides a composition comprising, consisting essentially of, or consisting of the above-described antibody, antibody fragment, such as a single chain polypeptide, or gene transfer vector and a pharmaceutically acceptable (e.g., physiologically acceptable) carrier, or an antibody or antigen binding fragment thereof optionally with a pharmaceutically acceptable (e.g., physiologically acceptable) carrier. When the composition consists essentially of the antibody, antibody fragment, e.g., single chain polypeptide, or gene transfer vector and a pharmaceutically acceptable carrier, additional components can be included that do not materially affect the composition (e.g., adjuvants, buffers, stabilizers, anti-inflammatory agents, solubilizers, preservatives, etc.). When the composition consists of the gene transfer vector and the pharmaceutically acceptable carrier, or the antibody, antigen binding fragment thereof or polypeptide optionally with a pharmaceutically acceptable carrier, the composition does not comprise any additional components. Any suitable carrier can be used within the context of the disclosure, and such carriers are well known in the art. The choice of carrier will be determined, in part, by the particular site to which the composition may be administered and the particular method used to administer the composition. The composition optionally can be sterile with the exception of the gene transfer vector or an antibody or antigen binding fragment thereof or polypeptide described herein. The composition can be frozen or lyophilized for storage and reconstituted in a suitable sterile carrier prior to use. The compositions can be generated in accordance with conventional techniques described in, e.g., Remington: The Science and Practice of Pharmacy, 21st Edition, Lippincott Williams & Wilkins, Philadelphia, PA (2001).

Suitable formulations for the composition include aqueous and non-aqueous solutions, isotonic sterile solutions, which can contain anti-oxidants, buffers, and bacteriostats, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets of the kind previously described. In one embodiment, the carrier is a buffered saline solution. In one embodiment, the gene transfer vector, antibody or antigen binding fragment thereof is administered in a composition formulated to protect the gene transfer vector or antibody or antigen binding fragment thereof from damage prior to administration. For example, the composition can be formulated to reduce loss of the gene transfer vector on devices used to prepare, store, or administer the gene transfer vector, such as glassware, syringes, or needles. The composition can be formulated to decrease the light sensitivity and/or temperature sensitivity of the gene transfer vector or an antibody or antigen binding fragment thereof. To this end, the composition may comprise a pharmaceutically acceptable liquid carrier, such as, for example, those described above, and a stabilizing agent selected from the group consisting of polysorbate 80, L-arginine, polyvinylpyrrolidone, trehalose, and combinations thereof. Use of such a composition will extend the shelf life of the gene transfer vector, facilitate administration, and increase the efficiency of the method. Formulations for gene transfer vector-containing compositions are further described in, for example, Wright et al., Curr. Opin. Drug Discov. Devel., 6 (2): 174-178 (2003) and Wright et al., Molecular Therapy, 12:171-178 (2005))

The composition also can be formulated to enhance transduction efficiency. In addition, one of ordinary skill in the art will appreciate that the gene transfer vector or antibody or antigen binding fragment thereof can be present in a composition with other therapeutic or biologically-active agents. For example, factors that control inflammation, such as ibuprofen or steroids, can be part of the composition to reduce swelling and inflammation associated with in vivo administration of the gene transfer vector or the antibody or antigen binding fragment thereof. Immune system stimulators or adjuvants, e.g., interleukins, lipopolysaccharide, and double-stranded RNA, can be administered to enhance or modify the anti-ART1 immune response. Antibiotics, i.e., microbicides and fungicides, can be present to treat existing infection and/or reduce the risk of future infection, such as infection associated with gene transfer procedures.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

In certain embodiments, a formulation of the present disclosure comprises a biocompatible polymer selected from the group consisting of polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, or copolymers thereof.

The composition can be administered in or on a device that allows controlled or sustained release, such as a sponge, biocompatible meshwork, mechanical reservoir, or mechanical implant. Implants (see, e.g., U.S. Pat. No. 5,443,505), devices (see, e.g., U.S. Pat. No. 4,863,457), such as an implantable device, e.g., a mechanical reservoir or an implant or a device comprised of a polymeric composition, are particularly useful for administration of the gene transfer vector, antibody or antigen binding fragement thereof. The composition also can be administered in the form of sustained-release formulations (see, e.g., U.S. Pat. No. 5,378,475) comprising, for example, gel foam, hyaluronic acid, gelatin, chondroitin sulfate, a polyphosphoester, such as bis-2-hydroxyethyl-terephthalate (BHET), and/or a polylactic-glycolic acid.

Delivery of the compositions comprising the gene transfer vectors, antibody or antigen binding fragment thereof or polypeptide, may be intracerebral (including but not limited to intraparenchymal, intraventricular, or intracisternal), intrathecal (including but not limited to lumbar or cisterna magna), or systemic, including but not limited to intravenous, oral, or any combination thereof, using devices known in the art. Delivery may also be via surgical implantation of an implanted device.

The dose of the gene transfer vector in the composition administered to the mammal will depend on a number of factors, including the size (mass) of the mammal, the extent of any side-effects, the particular route of administration, and the like. In one embodiment, the method comprises administering a “therapeutically effective amount” of the composition comprising the gene transfer vector, antibody or antigen binding fragment thereof described herein. A “therapeutically effective amount” refers to an amount effective, at dosages and for periods of time necessary, to achieve a desired therapeutic result. The therapeutically effective amount may vary according to factors such as the extent of pathology, age, sex, and weight of the individual, and the ability of the gene transfer vector, antibody or antigen binding fragment thereof to elicit a desired response in the individual. The dose of gene transfer vector in the composition required to achieve a particular therapeutic effect typically is administered in units of vector genome copies per cell (gc/cell) or vector genome copies/per kilogram of body weight (gc/kg). One of ordinary skill in the art can readily determine an appropriate gene transfer vector dose range to treat a patient having a particular disease or disorder, based on these and other factors that are well known in the art. A therapeutically effective amount may be between 1×10¹⁰ genome copies to 1×10¹³ genome copies. A therapeutically effective amount may be between 1×10¹² genome copies to 1×10¹⁵ genome copies (total). A therapeutically effective amount may be between 1×10¹² genome copies/kg to 1×10¹⁵ genome copies/kg.

The dose of antibody or antigen binding fragment thereof in the composition required to achieve a particular therapeutic effect typically is administered in units of antibody or antigen binding fragment per kg (mg/kg) or total dose (mg). One of ordinary skill in the art can readily determine an appropriate dose range to treat a patient having a particular disease or disorder, based on these and other factors that are well known in the art. A therapeutically effective amount of antibody or antigen binding fragment thereof may be between 25 to 200 mg, e.g., 50 to 100 mg, 25 to 50 mg, 50 to 75 mg, 100 to 150 mg, 150 to 200 mg, 200 mg to 300 mg, 300 mg to 400 mg, 400 mg to 500 mg, or 500 mg to 600 mg. A therapeutically effective amount of antibody or antigen binding fragment thereof may be between 1 mg/kg to 20 mg/kg, e.g., 2 to 5 mg/kg, 5 to 7 mg/kg or 10 to 15 mg/kg.

In one embodiment, the composition is administered once to the mammal. It is believed that a single administration of the composition will result in persistent expression of the anti-ART1 antibody in the mammal with minimal side effects. However, in certain cases, it may be appropriate to administer the composition multiple times during a therapeutic period to ensure sufficient exposure of cells to the composition. For example, the composition may be administered to the mammal two or more times (e.g., 2, 3, 4, 5, 6, 6, 8, 9, or 10 or more times) during a therapeutic period.

The present disclosure provides pharmaceutically acceptable compositions which comprise a therapeutically-effective amount of gene transfer vector comprising a nucleic acid sequence which encodes an antibody directed against ART1, or a therapeutically effective amount of the antibody or antigen binding fragment thereof, as described above.

### Exemplary Diseases and Conditions

Examples of diseases which may be prevented, inhibited or treated with the antibody or antigen binding fragment thereto includes but is not limited to neoplasms carcinomas (e.g., squamous-cell carcinomas, adenocarcinomas, hepatocellular carcinomas, and renal cell carcinomas), particularly those of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, particularly Ewing's sarcoma, hemangiosarcoma, Kaposi's sarcoma, liposarcoma, myosarcomas, peripheral neuroepithelioma, and synovial sarcoma; tumors of the central nervous system (e.g., gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas); germ-line tumors (e.g., bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, and melanoma); mixed types of neoplasias, particularly carcinosarcoma and Hodgkin's disease; and tumors of mixed origin, such as Wilms' tumor and teratocarcinomas (Beers and Berkow (eds.), The Merck Manual of Diagnosis and Therapy, 17.sup.th ed. (Whitehouse Station, N.J.: Merck Research Laboratories, 1999) 973-74, 976, 986, 988, 991. In one embodiment, the disease is pancreatic cancer, lung cancer, liver cancer, skin cancer, colon cancer, breast cancer, prostate cancer, leukemia, Burkitt like lymphoma, acute lymphoblastic leukemia or melanoma.

The disclosed compositions are useful to treat a subject with a medical condition or disorder that involves overexpression of ART1 or treat changes in ART1 activity, e.g., cancer.

### Subjects

The subject may be any animal, including a human and non-human animal. Non-human animals includes all vertebrates, e.g., mammals and non-mammals, such as non-human primates, sheep, dogs, cats, cows, horses, chickens, amphibians, and reptiles, although mammals are envisioned as subjects, such as non-human primates, sheep, dogs, cats, cows and horses. The subject may also be livestock such as, cattle, swine, sheep, poultry, and horses, or pets, such as dogs and cats.

Exemplary subjects include human subjects suffering from or at risk for the medical diseases and conditions described herein. The subject is generally diagnosed with the condition of the subject disclosure by skilled artisans, such as a medical practitioner.

The methods of the disclosure described herein can be employed for subjects of any species, gender, age, ethnic population, or genotype. Accordingly, the term subject includes males and females, and it includes elderly, elderly-to-adult transition age subjects adults, adult-to-pre-adult transition age subjects, and pre-adults, including adolescents, children, and infants.

Examples of human ethnic populations include Caucasians, Asians, Hispanics, Africans, African Americans, Native Americans, Semites, and Pacific Islanders. The methods of the disclosure may be more appropriate for some ethnic populations such as Caucasians, especially northern European populations, as well as Asian populations.

The term subject also includes subjects of any genotype or phenotype as long as they are in need of the disclosure, as described above. In addition, the subject can have the genotype or phenotype for any hair color, eye color, skin color or any combination thereof. The term subject includes a subject of any body height, body weight, or any organ or body part size or shape.

Exemplary Sequences
(SEQ ID NO: 1)
QVTLKESGPVLVKPTETLTLTCTVSGFSLSNARMGVSWIRQPPGKALEWLAHIFSNDEKSYSTSLKSRLTISKD
TSKSQVVLTMTNMDPVDTATYYCARIYGGDSWGYFDNWGQGTLVTVSS
(SEQ ID NO: 2)
QIVLTQSPAIMSASLGEKVTMTCSASSSVSYMHWYQQKSGTSPKRWIYDTSKLASGVPARFSGSGSGTSYSL
TISSMEAGDAATYYCQQWSSNPPTFGAGTKLELK
(SEQ ID NO: 3)
QVQLQESGPGLVKPSETLSLTCTVSGGSISSYYWSWIRQPAGKGLEWIGRISTSGFTNYNPSLKSRVTMSVD
SSKNQFSLKLSSLTAADTAVYYCARDGWGRVFDIWGLGTMVTVSS
(SEQ ID NO: 4)
EIVLTQSPGTLSLSPGERATLSCRASQSVSSSYLAWYQQKPGQAPRLLIYGASSRATGIPDRFSGSGSGTDFT
LTISRLEPEDFAVYYCQQYGSSTFGPGTKVDIK
(SEQ ID NO: 5)
CCTGCGCTTTCGGCCAGCG
(SEQ ID NO: 6)
CCAACAAAGTATACGCGGA
(SEQ ID NO: 7)
CGCGATAGCGCGAATATATT
(SEQ ID NO: 8)
TTG ATG ACC AGT ATG CTG GCT
(SEQ ID NO: 9)
TTG TTG GCC TGA AAG TCT GAG
(SEQ ID NO: 10)
CAG CGT CCT TTG ATG ACC AG
(SEQ ID NO: 11)
CCA CTG ATT GTT GGC CTG AG
(SEQ ID NO: 12)
AAT GTG TCC GTC GTG GAT CT
(SEQ ID NO: 13)
GGT CCT CAG TGT AGC CCA AG
(SEQ ID NO: 14)
AAG CTG ACC CTG AAG TTC ATC TGC
(SEQ ID NO: 15)
CTT GTA GTT GCC GTC GTC CTT GCC
(SEQ ID NO: 16)
MQMPAMMSLL LVSVGLMEAL QAQSHPITRR DLFSQEIQLD MALASFDDQY AGCAAAMTAA
LPDLNHTEFQ ANQVYADSWT LASSQWQERQ ARWPEWSLSP TRPSPPPLGF RDEHGVALLA
YTANSPLHKE FNAAVREAGR SRAHYLHHFS FKTLHFLLTE ALQLLGSGQR PPRCHQVFRG
VHGLRFRPAG PRATVRLGGF ASASLKHVAA QQFGEDTFFG IWTCLGAPIK GYSFFPGEEE
VLIPPFETFQ VINASRLAQG PARIYLRALG KHSTYNCEYI KDKKCKSGPC HLDNSAMGQS
PLSAVWSLLL LLWFLVVRAF PDGPGLL
(SEQ ID NO: 17)
TTTGATGTATTCACAGTTGTAT
(SEQ ID NO: 18)
TGCTGTTGACAGTGAGCGATAGACATCTTTTCTCAAGAAATAGTGAAGCCACAGATGTATTTCTTGAGAAA
AGATGTCTAGTGCCTACTGCCTCGGA.
(SEQ ID NO: 19)
CCAACAAAGTATACGCGGA
(SEQ ID NO: 20)
CGCGATAGCGCGAATATATT
(SEQ ID NO: 21)
NARMGVS
(SEQID NO: 22)
HIFSNDEKSYSTSLKS
(SEQ ID NO: 23)
IYGGDSWGYFDN
(SEQ ID NO: 24)
QVTLKESGPVLVKPTETLTLTCTVSGFSLS
(SEQ ID NO: 25)
WIRQPPGKALEWLA
(SEQ ID NO: 26)
RLTISKDTSKSQVVLTMTNMDPVDTATYYCAR
(SEQ ID NO: 27)
WGQGTLVTVSS
(SEQ ID NO: 28)
SSSVSY
(SEQ ID NO: 29)
DTS
(SEQ ID NO: 30)
QQWSSNPPT
(SEQ ID NO: 31)
QIVLTQSPAIMSASLGEKVTMTCSA
(SEQ ID NO: 32)
MHWYQQKSGTSPKRWIY
(SEQ ID NO: 33)
KLASGVPARFSGSGSGTSYSLTISSMEAGDAATYYC
(SEQ ID NO: 34)
FGAGTKLELK
(SEQ ID NO: 35)
GGSISSYY
(SEQ ID NO: 36)
ISTSGFT
(SEQ ID NO: 37)
ARDGWGRVFDI
(SEQ ID NO: 38)
QVQLQESGPGLVKPSETLSLTCTVS
(SEQ ID NO: 39)
WSWIRQPAGKGLEWIGR
(SEQ ID NO: 40)
NYNPSLKSRVTMSVDSSKNQFSLKLSSLTAADTAVYYC
(SEQ ID NO: 41)
WGLGTMVTVSS
(SEQ ID NO: 42)
QSVSSSY
(SEQ ID NO: 43)
GAS
(SEQ ID NO: 44)
QQYGSST.
(SEQ ID NO: 45)
EIVLTQSPGTLSLSPGERATLSCRAS
(SEQ ID NO: 46)
LAWYQQKPGQAPRLLIY
(SEQ ID NO: 47)
SRATGIPDRFSGSGSGTDFTLTISRLEPEDFAVYYC
(SEQ ID NO: 48)
DIQLTQSPSFLSASVGDRVTITCRASSSVSYMHWYQQKPGTSPKRLIYDTSKLASGVPSRFSGSGSGTEYTLT
ISSLQPEDFATYYCQQWSSNPPTFGQGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEVTHQGLSSPVTKSFNRGEC
(SEQ ID NO: 49)
DIQMTQSPSSLSASVGDRVTITCSASSSVSYMHWYQQKPGTSPKRLIYDTSKLASGVPSRFSGSGSGTDYTL
TISSLQPEDFATYYCQQWSSNPPTFGQGTKLEIKRTVAAPSVFIFPPSDEQLKSGTASVVCLLNNFYPREAKV
QWKVDNALQSGNSQESVTEQDSKDSTYSLSSTLTLSKADYEKHKVYACEVTHQGLSSPVTKSFNRGEC
(SEQ ID NO: 50)
EIVLTQSPATLSLSPGERATLSCRASSSVSYMHWYQQKPGTSPRRLIYDTSKLATGIPARFSGSGSGTDYTLTI
SSLEPEDFAVYYCQQWSSNPPTFGQGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEVTHQGLSSPVTKSFNRGEC
(SEQ ID NO: 51)
DIQLTQSPSFLSASVGDRVTITCRA
(SEQ ID NO: 52)
MHWYQQKPGTSPKRLIY
(SEQ ID NO: 53)
KLASGVPSRFSGSGSGTEYTLTISSLQPEDFATYYC
(SEQ ID NO: 54)
FGQGTKLEIKRTVAAPSVFIFPPSDEQLKSGTASVVCLLNNFYPREAKVQWKVDNALQSGNSQESVTEQDSK
DSTYSLSSTLTLSKADYEKHKVYACEVTHQGLSSPVTKSFNRGEC.
(SEQ ID NO: 55)
DIQMTQSPSSLSASVGDRVTITCSA
(SEQ ID NO: 56)
MHWYQQKPGTSPKRLIY
(SEQ ID NO: 57)
KLASGVPSRFSGSGSGTDYTLTISSLQPEDFATYYC
(SEQ ID NO: 58)
FGQGTKLEIKRTVAAPSVFIFPPSDEQLKSGTASVVCLLNNFYPREAKVQWKVDNALQSGNSQESVTEQDSK
DSTYSLSSTLTLSKADYEKHKVYACEVTHQGLSSPVTKSFNRGEC
(SEQ ID NO: 59)
EIVLTQSPATLSLSPGERATLSCRA
(SEQ ID NO: 60)
MHWYQQKPGTSPRRLIY
(SEQ ID NO: 61)
KLATGIPARFSGSGSGTDYTLTISSLEPEDFAVYYC
(SEQ ID NO: 62)
TFGQGTKLEIKRTVAAPSVFIFPPSDEQLKSGTASVVCLLNNFYPREAKVQWKVDNALQSGNSQESVTEQDS
KDSTYSLSSTLTLSKADYEKHKVYACEVTHQGLSSPVTKSFNRGEC
(SEQ ID NO: 63)
FGPGTKVDIK
(SEQ ID NO: 64)
QVTLKESGPVLVKPTETLTLTCTVSGFSLSNARMGVSWIRQPPGKALEWLAHIFSNDEKSYSTSLKSRLTIS
KDTSKSQVVLTMTNMDPVDTATYYCARIYGGDSWGYFDNWGQGTLVTVSSASTKGPSVFPLAPCSRSTSEST
AALGCLVKDYFPEPVTVSWNSGALTSGVHTFPAVLQSSGLYSLSSVVTVPSSSLGTKTYTCNVDKPSNTKVD
KRVESKYGPPCPPCPAPEFLGGPSVFLFPPKPKDTLMISRTPEVTCVVVDVSQEDPEVQFNWYVDGVEVHNA
KTKPREEQFNSTYRVVSVLTVLHQDWLNGKEYKCKVSNKGLPSSIEKTISKAKGQPREPQVYTLPPSQEEMT
KNQVSLTCLVKGFYPSDIAVEWESNGQPENNKTTPPVLDSDGSFFLYSRLTVDKSRWQYEGNVFSC SVM
HEALHNHYTQKSLSLSLGK
(SEQ ID NO: 65)
DIQLTQSPSFLSASVGDRVTITCRASSSVSYMHWYQQKPGTSPKRLIYDTSKLASGVPSRFSGSGSGTEYTLT
ISSLQPEDFATYYCQQWSSNPPTFGQGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEVTHQGLSSPVTKSFNRGEC
(SEQ ID NO: 66)
GFSLSNARMG
(SEQ ID NO: 67)
IFSNDEK
(SEQ ID NO: 68)
ARIYGGDSWGYFDN
(SEQ ID NO: 69)
DIQLTQSPSFLSASVGDRVTITCRASSSVSYMHWYQQKPGTSPKRLIYDTSKLASGVPSRFSGSGSGTEYTLT
ISSLQPEDFATYYCQQWSSNPPTFGQGTKL
(SEQ ID NO: 70)
SLSPTRPSPPPLGFRDEHGVALLAY
(SEQ ID NO: 71)
QVTLKESGPVLVKPTETLTLTCTVS
(SEQ ID NO: 72)
VSWIRQP PGKALEWLAH
(SEQ ID NO: 73)
SYSTSLKSRLTISKDTSKSQVVLTM TNMDPVDTATYYC
(SEQ ID NO: 74)
WGQGTLVTVSS
(SEQ ID NO: 75)
ASTKGPSVFPLAPCSRSTSESTAALGCLVKDYFPEPVTVSWNSGALTSGVHTFPAVLQSSGLYSLSSVVTVP
SSSLGTKTYTCNVDHKPSNTKVDKRVESKYGPPCPPCPAPEFLGGPSVFLFPPKPKDTLMISRTPEVTCVVV
DVSQEDPEVQFNWYVDGVEVHNAKTKPREEQFNSTYRVVSVLTVLHQDWLNGKEYKCKVSNKGLPSSIEKTI
SKAKGQPREPQVYTLPPSQEEMTKNQVSLTCLVKGFYPSDIAVEWE
SNGQPENNKTTPPVLDSDGSFFLYSRLTVDKSRWQYEGNVFSC SVM HEALHNHYTQKSLSLSLGK
(SEQ ID NO: 76)
DIQLTQSPSFLSASVGDRVTITCRAS
(SEQ ID NO: 77)
YMHWYQQKPGTS PKRLIY
(SEQ ID NO: 78)
KLASGVPSRFSGSGSGTEYTLTISSLQPEDFATY YC
(SEQ ID NO: 79)
FGQGTKLEIK
(SEQ ID NO: 80)
RTVAAPSVFIFPPSDEQLKSGT ASVVCLLNNFYPREAKVQWKVDNALQSGNSQESVTEQDSKDS TYSLSS
TLTLSKADYEKHKVYACEVTHQGLSSPVTKSFNRGEC
(SEQ ID NO: 81)
SSVSY
(SEQ ID NO: 82)
QVTLKESGPVLVKPTETLTLTCTVSGFSLSNARMGVSWIRQPPGKALEWLAHIFSNDEKSYSTSLKSRLTISKD
TSKSQVVLTMTNMDPVDTATYYCARIYGGDSWGYFDNWGQGTLVTVSS
(SEQ ID NO: 83)
DIQLTQSPSFLSASVGDRVTITCRASSSVSYMHWYQQKPGTS PKRLIYDTS
KLASGVPSRFSGSGSGTEYTLTISSLQPEDFATYYCQQWSSNPPTFGQGTKLEIK.
(SEQ ID NO: 84)
DIQMTQSPSSLSASVGDRVTITCSASSSVSYMHWYQQKPGTSPKRLIYDTSKLASGVPSRFSGSGSGTDYTL
TISSLQPEDFATYYCQQWSSNPPTFGQGTKL
(SEQ ID NO: 85)
AGA AGA GGT CTC GTC GTG TGA
(SEQ ID NO: 86)
GAT GCC TGC TAT GAT GTC TCT G
(SEQ ID NO: 87)
EIVLTQSPATLSLSPGERATLSCRASSSVSYMHWYQQKPGTSPRRLIYDTSKLATGIPARFSGSGSGTDYTLTI
SSLEPEDFAVYYCQQWSSNPPTFGQGTKL.
(SEQ ID NO: 88)
MKIPAMMSLL LVSVGLRDGV QVQSYSISQL DIFSQETPLD MAPASFDDQY AGCLADMTAA LPDLNHSEFQ
ANKVYADGWA QANNQWQERR AWGSVWGSLP PSPPGFRDEH GVALLAYTAN SPLHKEFNAA
VREAGRSRAH YLHHFSFKTL HFLLTEALQL LRSHRSRGCQ QVYRGVHGLR FRPAGPGATV
RLGGFASASL KNVAAQQFGE DTFFGIWTCL GAPIRGYSFF PEEEEVLIPP
FETFQVINTS RPTQGPARIY LRALGKRSTY NCEYIKEKKC RSGPCWLGSS APGSISASCS
LLLLLLFLVL SALPENPGLQ QLTRC.
(SEQ ID NO: 90)
MQMPAMMSLLLVSVGLMEALQAQSHPITRRDLFSQEIQLDMALASFDDQYAGCAAAMTAA
LPDLNHTEFQANQVYADSWTLASSQWQERQARWPEWSLSPTRPSPPPLGFRDEHGVALLA
YTANSPLHKEFNAAVREAGRSRAHYLHHFSFKTLHFLLTEALQLLGSGQRPPRCHQVFRG
VHGLRFRPAGPRATVRLGGFASASLKHVAAQQFGEDTFFGIWTCLGAPIKGYSFFPGEEE
VLIPPFETFQVINASRLAQGPARIYLRALGKHSTYNCEYIKDKKCKSGPCHLDNSAMG
(SEQ ID NO: 91)
M++PAMMSLLLVSVGL + +Q QS+ I++ D+FSQE LDMA ASFDDQYAGC A MTAA
LPDLNH+EFQAN+VYAD WA++ QWQER+A W PSPP GFRDEHGVALLA
YTANSPLHKEFNAAVREAGRSRAHYLHHFSFKTLHFLLTEALQLL S R C QV+RG
VHGLRFRPAGP ATVRLGGFASASLK+VAAQQFGEDTFFGIWTCLGAPI+GYSFFP EEE
VLIPPFETFQVIN SR QGPARIYLRALGK STYNCEYIK+KKC+SGPC L +SA G
(SEQ ID NO: 92)
MKIPAMMSLLLVSVGLRDGVQVQSYSISQLDIFSQETPLDMAPASFDDQYAGCLADMTAA
LPDLNHSEFQANKVYADGWAQANNQWQERRAWGSVWGSLP--PSPP--GFRDEHGVALLA
YTANSPLHKEFNAAVREAGRSRAHYLHHFSFKTLHFLLTEALQLLRS-HRSRGCQQVYRG
VHGLRFRPAGPGATVRLGGFASASLKNVAAQQFGEDTFFGIWTCLGAPIRGYSFFPEEEE
VLIPPFETFQVINTSRPTQGPARIYLRALGKRSTYNCEYIKEKKCRSGPCWLGSSAPG
(SEQ ID NO: 93)
NAHMGVS
(SEQ ID NO: 94)
QARMGIS
(SEQ ID NO: 95)
NGRMGVS
(SEQ ID NO: 96)
HIFSNDEKSYSTSIKS
(SEQ ID NO: 97)
HLFSNDEKSYSTSIKS
(SEQ ID NO: 98)
HIFTNDEKSYSSSLKS
(SEQ ID NO: 99)
IYGGADSWGYFEN
(SEQ ID NO: 100)
IYGGDSWAYFDN
(SEQ ID NO: 101)
LYGIDSWGYFDN
(SEQ ID NO: 102)
GFSISNARMG
(SEQ ID NO: 103)
GFSASNTRMG
(SEQ ID NO: 104)
GFSISNLRMA.
(SEQ ID NO: 105)
LFSNDEK
(SEQ ID NO: 106)
IFSNEDK.
(SEQ ID NO: 107)
GRIYGGDSWGYFDN
(SEQ ID NO: 108)
ARIYAADSWGYFDN
(SEQ ID NO: 109)
IRAYGGDSWLYFDN

### EXEMPLARY EMBODIMENTS

The disclosure provides an isolated cell comprising an expression cassette comprising a heterologous promoter operably linked to nucleic acid sequences encoding an anti-human ART1 antibody, or an antigen binding fragment thereof, or a polypeptide, that inhibits human ART1 activity, wherein the antibody, the antigen binding fragment thereof, or the polypeptide has: a variable heavy chain region comprising a first complementarity determining region (CDR) operably linked to a second CDR operably linked to a third CDR comprising; and/or a variable light region comprising a first CDR operably linked to a second CDR operably linked to a third CDR. The sequences in the CDRs may include any of SEQ ID Nos. 21-23, 28-30, 35-36, 42-44, 66-68, or 81, or a sequence with 1, 2, 3, 4 or 5 substitutions. In one embodiment, the majority or all of the substitutions are conversative substitutions. In one embodiment, the heavy or light chain may include one or more framework regions including but not limited to one of SEQ ID Nos. 24-27, 31-34, 38-41, 45-47, 51-63, 71-74, or 76-79, or a sequence with 1, 2, 3, 4, 5, 6, 7, 8, 9 or 10 substitutions. In one embodiment, the majority or all of the substitutions are conversative substitutions. In one embodiment, the heavy or light chain may include one or more framework regions including but not limited to one of SEQ ID Nos. 24-27, 31-34, 38-41, 45-47, 51-63, 71-74, or 76-79, or a sequence having at least 80%, 85%, 90%, 92%, 95% or 99% amino acid sequence identity thereto. In one embodiment, the cell is a mammalian cell, e.g., a primate cell or a rodent cell, e.g., a CHO cell. In one embodiment, the cell is a human cell.

Also provided is a hybridoma comprising nucleic acid sequences encoding an anti-human ART1 monoclonal antibody that inhibits human ART1 activity, wherein the antibody has: a variable heavy chain region comprising a first CDR operably linked to a second CDR operably linked to a third CDR comprising; and/or a variable light region comprising a first CDR operably linked to a second CDR operably linked to a third CDR. The sequences in the CDRs may include any of SEQ ID Nos. 21-23, 28-30, 35-37, 42-44, 66-68, or 81, or a sequence with 1, 2, 3, 4 or 5 substitutions. In one embodiment, the majority or all of the substitutions are conversative substitutions. In one embodiment, the heavy or light chain may include one or more framework regions including but not limited to one of SEQ ID Nos. 24-27, 31-34, 38-41, 45-47, 51-63, 71-74, or 76-79, or a sequence with 1, 2, 3, 4, 5, 6, 7, 8, 9 or 10 substitutions. In one embodiment, the heavy or light chain may include one or more framework regions including but not limited to one of SEQ ID Nos. 24-27, 31-34, 38-41, 45-47, 51-63, 71-74, or 76-79, or a sequence having at least 80%, 85%, 90%, 92%, 95% or 99% amino acid sequence identity thereto.

Further provided is an isolated nucleic acid comprising a promoter operably linked to a nucleotide sequence which encodes at least the variable region of a heavy or light Ig chain that binds human and/or mouse ART1, wherein the chain comprises: a variable heavy chain region comprising a first CDR operably linked to a second CDR operably linked to a third CDR comprising; and/or a variable light region comprising a first CDR operably linked to a second CDR operably linked to a third CDR. The sequences in the CDRs may include any of SEQ ID Nos. 21-23, 28-30, 35-36, 42-44, 66-68, or 81, or a sequence with 1, 2, 3, 4 or 5 substitutions. In one embodiment, the majority or all of the substitutions are conversative substitutions. In one embodiment, the heavy or light chain may include one or more framework regions including but not limited to one of SEQ ID Nos. 24-27, 31-34, 38-41, 45-47, 51-63, 71-74, or 76-79, or a sequence with 1, 2, 3, 4, 5, 6, 7, 8, 9 or 10 substitutions. In one embodiment, the heavy or light chain may include one or more framework regions including but not limited to one of SEQ ID Nos. 24-27, 31-34, 38-41, 45-47, 51-63, 71-74, or 76-79, or a sequence having at least 80%, 85%, 90%, 92%, 95% or 99% amino acid sequence identity thereto.

Also provided is an isolated antibody or antigen fragment thereof that binds human and mouse ART1, wherein the antibody or the antigen binding fragment thereof has: a variable heavy chain region comprising a first CDR operably linked to a second CDR operably linked to a third CDR comprising; and/or a variable light region comprising a first CDR operably linked to a second CDR operably linked to a third CDR. The sequences in the CDRs may include any of SEQ ID Nos. 21-23, 28-30, 35-36, 42-44, 66-68, or 81, or a sequence with 1, 2, 3, 4 or 5 substitutions. In one embodiment, the majority or all of the substitutions are conversative substitutions. In one embodiment, the heavy or light chain may include one or more framework regions including but not limited to one of SEQ ID Nos. 24-27, 31-34, 38-41, 45-47, 51-63, 71-74, or 76-79, or a sequence with 1, 2, 3, 4, 5, 6, 7, 8, 9 or 10 substitutions. In one embodiment, the heavy or light chain may include one or more framework regions including but not limited to one of SEQ ID Nos. 24-27, 31-34, 38-41, 45-47, 51-63, 71-74, or 76-79, or a sequence having at least 80%, 85%, 90%, 92%, 95% or 99% amino acid sequence identity thereto.

Inhibitors of ART1, e.g., an anti-human ART1 antibody or an antigen binding fragment thereof, or a polypeptide that binds human ART1, may be employed in vivo. In one embodiment, a method to inhibit or treat cancer in a mammal is provided in which a composition comprising an effective amount of an anti-human ART1 antibody or an antigen binding fragment thereof, or a polypeptide that binds human ART1, is administered to a mammal. In one embodiment, the cancer is lung cancer, colon cancer, melanoma, glioblastoma, breast cancer or colorectal cancer. In one embodiment, the mammal is a human. In one embodiment, the amount is effective to decrease tumor burden, inhibit metastases, increase survival, or any combination thereof. In one embodiment, the composition is systemically administered. In one embodiment, the mammal is also administered a chemotherapeutic drug. In one embodiment, the mammal is administered an immune checkpoint inhibitor. In one embodiment, the heavy chain of the antibody or fragment thereof is an IgG heavy chain. In one embodiment, the light chain of the antibody or fragment thereof is an Igκ light chain. In one embodiment, the fragment is Fab′, F(ab′)2, scFv or a single domain. In one embodiment, the antibody or the antigen binding fragment thereof, or the polypeptide, has: a variable heavy chain region comprising a first CDR operably linked to a second CDR operably linked to a third CDR comprising; and/or a variable light region comprising a first CDR operably linked to a second CDR operably linked to a third CDR. The sequences in the CDRs may include any of SEQ ID Nos. 21-23, 28-30, 35-36, 42-44, 66-68, or 81, or a sequence with 1, 2, 3, 4 or 5 substitutions. In one embodiment, the majority or all of the substitutions are conversative substitutions. In one embodiment, the heavy or light chain may include one or more framework regions including but not limited to one of SEQ ID Nos. 24-27, 31-34, 38-41, 45-47, 51-63, 71-74, or 76-79, or a sequence with 1, 2, 3, 4, 5, 6, 7, 8, 9 or 10 substitutions. In one embodiment, the heavy or light chain may include one or more framework regions including but not limited to one of SEQ ID Nos. 24-27, 31-34, 38-41, 45-47, 51-63, 71-74, or 76-79, or a sequence having at least 80%, 85%, 90%, 92%, 95% or 99% amino acid sequence identity thereto.

In one embodiment, a method to prevent, inhibit or treat ART1-mediated immunosuppression in a mammal, is provided. The method includes administering to a mammal a composition comprising an effective amount of an anti-human ART1 antibody or an antigen binding fragment thereof, or a polypeptide that binds human ART1. In one embodiment, the composition is systemically administered. In one embodiment, the mammal is also administered a chemotherapeutic drug and/or radiation therapy. In one embodiment, the mammal is administered an immune checkpoint inhibitor. In one embodiment, the heavy chain of the antibody or fragment thereof is an IgG heavy chain. In one embodiment, the light chain of the antibody or fragment thereof is an Igκ light chain. In one embodiment, the fragment is Fab′ or scFv. In one embodiment, the antibody or the antigen binding fragment thereof, or the polypeptide, has: a variable hevy chain region comprising a first CDR operably linked to a second CDR operably linked to a third CDR comprising; and/or a variable light region comprising a first CDR operably linked to a second CDR operably linked to a third CDR. The sequences in the CDRs may include any of SEQ ID Nos. 21-23, 28-30, 35-36, 42-44, 66-68, or 81, or a sequence with 1, 2, 3, 4 or 5 substitutions. In one embodiment, the majority or all of the substitutions are conversative substitutions. In one embodiment, the heavy or light chain may include one or more framework regions including but not limited to one of SEQ ID Nos. 24-27, 31-34, 38-41, 45-47, 51-63, 71-74, or 76-79, or a sequence with 1, 2 3, 4, 5, 6, 7, 8, 9 or 10 substitutions. In one embodiment, the heavy or light chain may include one or more framework regions including but not limited to one of SEQ ID Nos. 24-27, 31-34, 38-41, 45-47, 51-63, 71-74, or 76-79, or a sequence having at least 80%, 85%, 90%, 92%, 95% or 99% amino acid sequence identity thereto.

Also provided is a method to enhance an immune response in a mammal having cancer, comprising: administering to a mammal a composition comprising an effective amount of an anti-human ART1 antibody or an antigen binding fragment thereof, or a polypeptide that binds human ART1. In one embodiment, the composition is systemically administered. In one embodiment, the mammal is also administered a chemotherapeutic drug and/or radiation therapy. In one embodiment, the mammal is administered an immune checkpoint inhibitor. In one embodiment, the heavy chain of the antibody or fragment thereof is an IgG heavy chain. In one embodiment, the light chain of the antibody or fragment thereof is an Igκ light chain. In one embodiment, the fragment is Fab′ or scFv. In one embodiment, the antibody or the antigen binding fragment thereof, or the polypeptide, has: a variable hevy chain region comprising a first CDR operably linked to a second CDR operably linked to a third CDR comprising; and/or a variable light region comprising a first CDR operably linked to a second CDR operably linked to a third CDR. The sequences in the CDRs may include any of SEQ ID Nos. 21-23, 28-30, 35-36, 42-44, 66-68, or 81, or a sequence with 1, 2, 3, 4 or 5 substitutions. In one embodiment, the majority or all of the substitutions are conversative substitutions. In one embodiment, the heavy or light chain may include one or more framework regions including but not limited to one of SEQ ID Nos. 24-27, 31-34, 38-41, 45-47, 51-63, 71-74, or 76-79, or a sequence with 1, 2 3, 4, 5, 6, 7, 8, 9 or 10 substitutions. In one embodiment, the heavy or light chain may include one or more framework regions including but not limited to one of SEQ ID Nos. 24-27, 31-34, 38-41, 45-47, 51-63, 71-74, or 76-79, or a sequence having at least 80%, 85%, 90%, 92%, 95% or 99% amino acid sequence identity thereto.

The invention will be described by the following non-limiting examples.

### Example 1

To determine whether ART1 contributes to distinct phenotypic characteristics, ART1 expression was knocked down in murine KRAS^{G12D/+}/p53^{-/-} cell lines (KP1) using shRNAs. In both tail vein injection induced lung and subcutaneously injected flank tumor models in immunocompetent mice, a significant decrease in tumor burden was noted upon ART1-knockdown and enzymatic inhibition. In the KP1 tail vein model, ART1 knockdown is associated (p<0.001) with control of tumor progression and with long term survival (approximately 50%) of the mice, superior to that seen with immune checkpoint blockade (ICB) using a PD-1 antibody. This effect of ART1 knockdown on tumor growth was significantly abrogated in nude mice and CD8-depleted mice, suggesting that ART1 may act through immune-mediated mechanisms. ART1 may specifically MAR P2RX7 on CD8+ T cells, in particular tissue resident memory T cells (Trm). P2RX7 is a cytolytic ATP receptor that mediates apoptotic cell death of T cells, macrophages, and dendritic cells. Upon cellular stress or death and release of extracellular NAD⁺, MAR of P2RX7 on the surface of T cells results in prolonged receptor activation, Ca²⁺ influx, and T cell apoptosis, a phenomenon termed NAD-induced cell death (NICD). Thus it was hypothesized that ART1-induced MAR of P2RX7 on T cells could allow ART1-high cancer cells to blunt the T cell immune response against them by inducing T cell apoptosis. Indeed, NAD⁺ released during cell death and inflammation has previously been demonstrated to regulate homeostasis of cytotoxic CD8 T cells through extracellular ADP-ribosylation. By RNAseq of lung-infiltrating CD8 T cell populations in murine models, we showed that the relative expression of P2RX7 increases in CD8+ T cells with increasing tumor burden following tail vein injection in mice (p<0.001) and that P2X7R+ CD8 T cells are preferentially ADP-ribosylated. In vivo, using a dox-inducible shRNA, it was demonstrated that higher ART1 expression in cancer cells leads to a lower proportion of activated, lung infiltrating CD8+ T cells. Similar findings were observed in ART1-overexpressing human tumors, in which it was found that a lower proportion of CD103+/P2RX7+ CD8+ Trm cells than in adjacent lung with lower ART1. In murine models, we observed that abrogation of ART1 expression in cancer cells leads to significant enrichment of tumor-infiltrating P2RX7+/CD8+ and CD103+/CD8+ (Trm) T cells. Additionally, it was observed that there was a significant enrichment of activated CD103+/P2RX7+ dendritic cells (DC) in KP1-implanted tumor mice upon knockdown of ART1.

The complex relationship between ART1-expressing cancer cells and the tumor immune microenvironment was studied. As an extracellular, enzymatic target, ART1 should be highly druggable. MAR can be blocked by small molecule arginine analogues such as the antibiotic novobiocin, MIBG (a safe norepinephrine analogue imaging agent), and nonspecific PARP inhibitors (like EB-47). These drugs compete with NAD+ at the enzyme active site and have been shown to have anti-cancer effects in murine models or in untargeted patient populations. ART1-overexpressing tumors can be targeted with therapeutic monoclonal antibodies, similar to ICB. A humanized therapeutic monoclonal antibody (22C12) which binds to ART1 and inhibits ADP-ribosylation on ART1-expressing cancer cells was developed (see Examples 2 and 3). Preliminary data demonstrated a drastic reduction (˜59%) of murine KP1 flank tumors when intratumorally treated with 22C12 compared to control antibody. This reduction was associated with significant enrichment of P2X7R+ CD8+ T cells and P2RX7+CD103+ DC within the tumors.

The mechanisms and appropriate context for therapeutic targeting of ART1 were investigated. ART1 expression may be upregulated by cell stress and its enzymatic activity increased by release of NAD+, both of which occur following commonly used cytotoxic therapies. Thus, increases of tumor ART1 expression post-treatment may serve as the basis for therapeutic inhibition of ART1 combined with chemotherapy, radiation therapy, or immune checkpoint blockade (ICB).

### Example 2

### Development of a Therapeutic Antibody Targeting ART1, an Extracellular Mono-ADP Ribosyltransferase, for the Treatment of Cancer

Antibodies that specifically inhibit ART1 or its function may be used as targeted therapeutics in ART1-expressing, e.g., overexpressing, cancers such as in ART-1 overexpressing NSCLC patients to limit metastatic spread of cancer by facilitating immune-mediated destruction of disseminated cells. As an extracellular enzymatic target, ART1 is highly druggable. ART1 antibodies may be used as combination therapy with cytotoxic chemotherapy or with immune checkpoint inhibitors.

In one embodiment, the antibody is reactive against both mouse and human ART1. Antibodies are enzymatically screened for inhibitors of mono-ADP-ribosylation utilizing ART1, substrate proteins, and labeled NAD+. Potential therapeutic antibodies are tested utilizing in vitro neutrophil and lymphocyte cytotoxicity assays. In vitro, therapeutic blockade of ART1 facilitates cytotoxicity of the immune cells towards the ART1 expressing lung cancer cells. Notably, ART1 expression (and a similar phenotype) was observed in murine and human breast cancer models and in clinical specimens, as well as in human colorectal cancer specimens. Thus, the anti-ART1 antibodies have broad anti-cancer applicability.

To generate functional humanized antibodies against ART1, purified recombinant human and mouse ART1 produced in HEK293 mammalian cells was used to immunize transgenic mice with a human immunoglobulin repertoire. Following immunization and test bleed analysis, spleens were harvested and fused with a myeloma fusion partner to generate hybridoma.

First, hybridoma supernatants were screened on ELISA plates coated with purified human ART1. Hybridoma cells from positive wells were frozen for later recovery. Anti-human ART1 positive hybridoma supernatants were then tested for inhibition of purified human ART1 by a fluorescent NAD⁺ readout. This assay measures NAD⁺ consumed from ADP-ribosylation of histone protein by ART1.

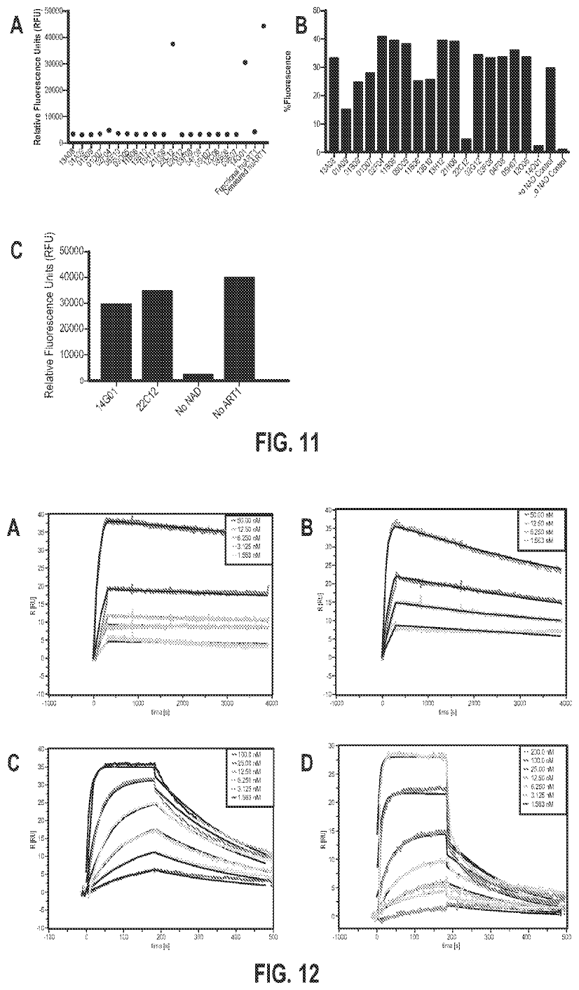

Anti-ART1 hybridoma supernatants were also tested for inhibition of human ART1 transiently expressed in HEK293 cells. This method, which has previously been described for ART2 (Krebs et al.), determines cell-surface ADP-ribosylation via an NAD⁺ analogue (etheno-NAD⁺) which is then detected by an anti-etheno antibody using flow cytometry. Two hybridoma supernatants (fusion wells 22C12 and 14G01) were positive for inhibition of human ART1 in the biochemical and cell-based assays. Supernatants from 22C12 and 14G01 were then also tested for binding to moART1 by ELISA and inhibition of purified mouse ART1 (FIG. 11). 27 hybridoma clones secreted antobodies that bound to recombinant human ART1, 2 clones secreted antibodies that inhibited recombinant human ART1, 2 clones secreted antibodies that inhibited human ART expressed on HEK293 cells, 2 clones secreted antibodies that bound to mouse ART1, and 2 clones secreted antibodies that inhibited mouse ART1.

Following hybridoma subcloning and expansion of clones 22C12 and 14G01, antibodies were purified from hybridoma supernatant for potency ranking and affinity determination by surface plasmon resonance (SPR). Purified antibodies from clones 22C12 and 14G01 were captured on an SPR anti-Fc chip and purified human and mouse ART1 was used as analyte at the indicated concentrations (FIG. 12).

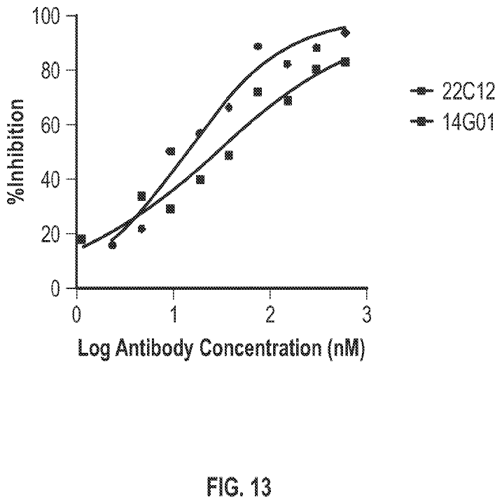

To determine potency in the cell-based functional assay, purified antibodies from clones 22C12 and 14G01 were incubated with HEK293 cells transiently transfected with huART1 at the indicated concentrations prior to treatment with etheno-NAD⁺. Cell-surface ADP-ribosylation was then determined by flow cytometry and used to calculate IC₅₀ values (FIG. 13). The binding affinity and in vitro potency data from mAbs 22C12 and 14G01 are summarized in Table 1.

TABLE 1
Binding Affinity and in vitro Potency
Data for lead candidate mAbs

| | KD Human ART1 | KD Mouse ART1 | IC₅₀ Cell Surface |
|---|---|---|---|
| Clone | (SPR) | (SPR) | ADP-Ribosylation |
| 22C12 | 161 pM | 439 pM | 14 nM |
| 14G01 | 4 nM | 6 nM | 28 nM |

pM: picomolar
nM: nanomolar

### Exemplary ART1 Antibody Sequences:

22C12 VH (human)
(SEQ ID NO: 1)
QVTLKESGPVLVKPTETLTLTCTVSGFSLSNARMGVSWIRQPPGKALE
WLAHIFSNDEKSYSTSLKSRLTISKDTSKSQVVLTMTNMDPVDTATYY
CARIYGGDSWGYFDNWGQGTLVTVSS
22C12 VK (mouse)
(SEQ ID NO: 2)
QIVLTQSPAIMSASLGEKVTMTCSASSSVSYMHWYQQKSGTSPKRWI
YDTSKLASGVPARFSGSGSGTSYSLTISSMEAGDAATYYCQQWSSNP
PTFGAGTKLELK
14G01 VH (human)
(SEQ ID NO: 3)
QVQLQESGPGLVKPSETLSLTCTVSGGSISSYYWSWIRQPAGKGLEW
IGRISTSGFTNYNPSLKSRVTMSVDSSKNQFSLKLSSLTAADTAVYY
CARDGWGRVFDIWGLGTMVTVSS
14G01 VK (human)
(SEQ ID NO: 4)
EIVLTQSPGTLSLSPGERATLSCRASQSVSSSYLAWYQQKPGQAPRL
LIYGASSRATGIPDRFSGSGSGTDFTLTISRLEPEDFAVYYCQQYGS
STFGPGTKVDIK

Red: CDRs are labeled according to IMGT nomenclature

### Example 3

### ART1 Tilts the Balance of Life and Death for Anti-Tumor T Cells

Elimination of T cells through NAD-induced cell death following mono-ADP-ribosylation of the P2X7-receptor constitutes a regulatory mechanism to maintain tissue immune homeostasis. The present disclosure demonstrates that, in non-small cell lung cancer, tumor cells overexpressing the mono-ADP-ribosyltransferase 1, ART1, coopt this mechanism to escape immune-mediated control by eliminating P2X7-receptor-positive CD8 T cells in the local microenvironment. Therapeutic targeting of ART1 with a novel monoclonal antibody improved tumor control in mouse models of lung cancer.

In particular, it was found that ART1 was strongly expressed in the majority of lung adenocarcinomas and that its membrane expression was associated with lower CD8 T cell infiltration compared to ART1 negative tumors. In a murine model of NSCLC, it was demonstrated that genetic and pharmacologic targeting of ART1 inhibited tumor growth in immunocompetent but not in immunodeficient mice and increased infiltration of P2X7R⁺CD8 T cells in tumors in the immunocompetent mice. In vitro experiments confirmed that CD8 T cells isolated from wild type mice, but not P2X7R^{-/-} mice, were preferentially susceptible to ART1-mediated ADP-ribosylation and NICD. Expression of the ADP-ribosyl cyclase CD38, which degrades NAD⁺, by P2X7R⁺ CD8 T cells reduced ART1-mediated ADP-ribosylation and NICD. ART1-mediated NICD is a mechanism of immune resistance in NSCLC and other cancers.

### Introduction

Immune checkpoint inhibitors (ICI), alone or in combination with chemotherapy, have become the standard of care in patients with advanced non-small cell lung cancer (NSCLC) without targetable molecular alterations (Mok et al., 2019; Reck et al., 2016). However, the majority of lung cancer patients either do not respond to or do not experience long-term benefit from ICI, including many of those patients with high tumor PD-L1 expression (Gandhi et al., 2018; Gandini et al., 2016). Thus, there is an urgent need to identify other robust biomarkers predictive of response to ICI, and to understand the mechanisms of primary and acquired resistance of lung cancer to immunotherapy.

In humans, ADP-ribosyltransferase-1 (ART1) is expressed at low levels in healthy tissues including the lung. ART1 is a GPI-anchored enzyme, with an extracellular catalytic domain. Therefore, ART1 can mono-ADP-ribosylate extracellular proteins in the local microenvironment, altering their function (Stevens et al., 2009; Okazaki et al., 1994; Balducci et al., 1999). The expression of ART1 in lung cancer has not been investigated, but previous studies have suggested increased ART1 protein expression in colorectal cancer and in glioblastoma, where high expression was associated with a poor prognosis (Yang et al., 2013). In mouse models of colorectal cancer, ART1 expression was shown to promote a more aggressive phenotype with increased epithelial-to-mesenchymal transition, cellular proliferative signaling and increased angiogenesis (Yang et al., 2013; Song et al., 2016). However, it has not been determined whether tumor ART1 expression could regulate tumor crosstalk with the immune microenvironment.

Among the well described targets of ADP-ribosyl transferases is the P2X7 receptor (P2X7R, gene id: P2RX7). P2X7R is an ATP-gated cation channel of the purinergic type 2 receptor family, with low affinity for extracellular ATP, that activates pro-inflammatory pathways (Burnstalk & Knight, 2004). It is expressed on multiple immune cell subsets including T cells and its expression is essential for inflammatory responses and anti-tumor immunity (Adinolfi et al., 2015; Haag et al., 2007). P2X7R can also be overexpressed on cancer cells where it may promote tumor progression. However, in NSCLC, high P2X7R expression has been associated with improved overall and progression-free survival (Boldrini et al., 2015). In pathological conditions such as tissue damage, tumor development, or inflammation, cytosolic NAD⁺ is released into the local extracellular environment where it may be used as a substrate by extracellular ADP-ribosyl transferases to catalyze the transfer of the ADP-ribose to P2X7R (Haag et al., 2007). This covalent modification results in constitutive activation of P2X7R triggering large pore formation, uncontrolled calcium influx, and phosphatidylserine externalization, which leads to a process known as NAD-induced cell death (NICD) (Scheuplein et al., 2009). Typically, extracellular NAD⁺ concentrations are low and tightly regulated by the ADP-ribosyl cyclase CD38, which is expressed on activated immune cells as well as on cancer cells (Sandoval-Montes & Santos-Argumedo, 2005; Chen et al., 2018). However, even in the presence of CD38, extracellular NAD⁺ concentrations can increase following rapid release from stressed or dying cells (Haag et al., 2007). In preclinical studies, ART-mediated NICD of T cells has been proposed as a homeostatic mechanism to eliminate naïve and bystander T cells in inflamed tissues (Adriouch et al., 2007). More recently, NICD was shown to regulate the homeostasis of CD4 regulatory T cells (CD4 Tregs) which have broad immunoregulatory function as well as tissue-resident memory T cells (TRMs), the presence of which in lung tumors has been associated with good prognosis (Stark et al., 2018; Nizard et al., 2017). These pre-clinical studies discern the role of ADP-ribosyltransferase-2 (ART2) in immune modulation through NICD. ART2 is expressed on murine lymphocytes where it can auto-ADP-ribosylate the P2X7R mediating NICD in cis. However, in humans, the ART2 gene contains premature stop codons rendering it a pseudogene while other ARTs like ART1, ART3, ART4 and ART5 are transcriptionally active.

As disclosed herein, ART1 is expressed on the surface of human lung cancer cells and that its expression is associated with reduced lung tumor infiltration of P2X7R⁺ CD8 T cells. In preclinical models of lung cancer and melanoma, tumor cell ART1 expression promoted escape from CD8 T cell-mediated tumor control. ART1-blockade with a therapeutic monoclonal antibody reduced the growth and dissemination of ART1-expressing tumors in immunocompetent mice and promoted tumor infiltration of activated P2X7R+CD8 T cells. Overall, the data suggest that ART1 tumor expression is a unique mechanism of immune resistance and that ART1 is an actionable target to enhance T cell-mediated tumor rejection.

### Results

ART1 is Expressed in Human NSCLC and Associated with Reduced CD8 T Cell Infiltration

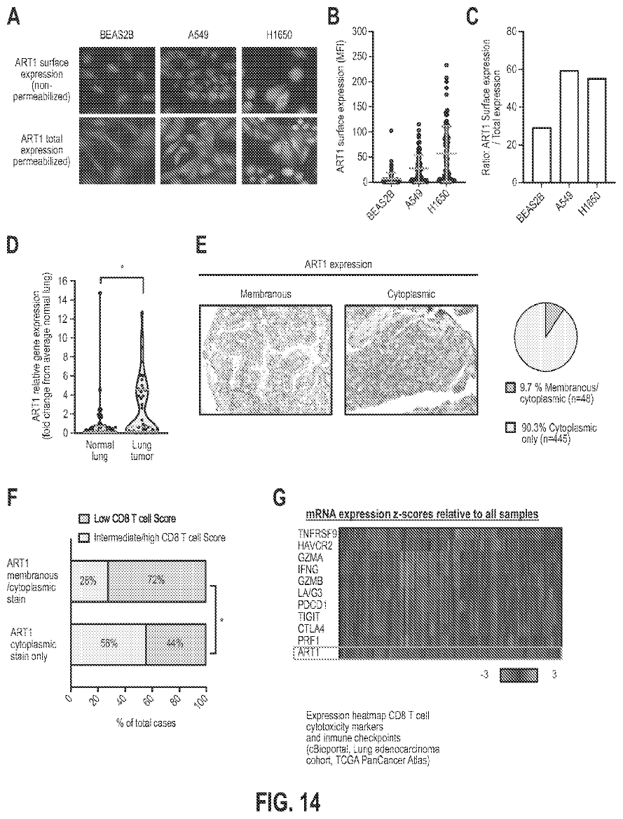

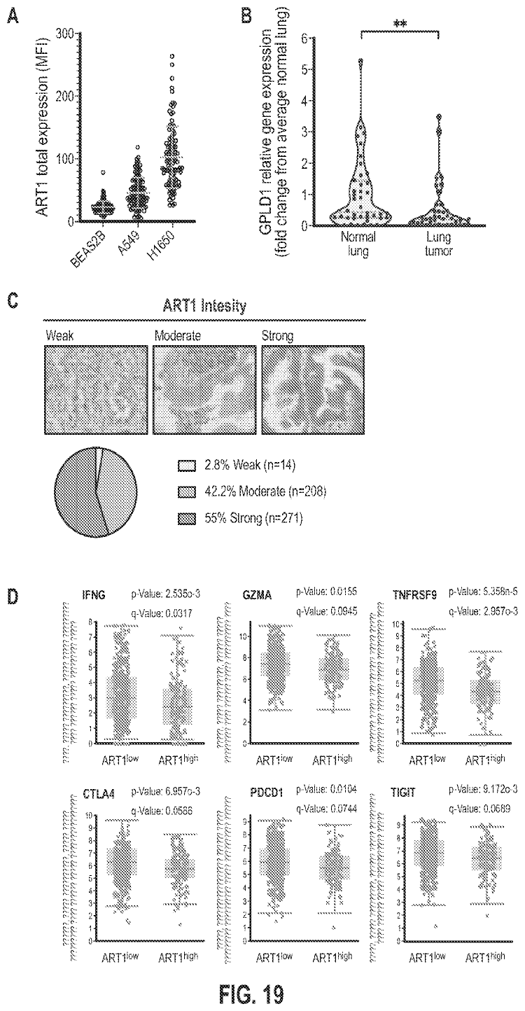

ART1 expression was assessed in human NSCLC lines A549 and H1650 and in a benign bronchial epithelial cell line (BEAS2B) by immunofluorescence. The tumor cell lines had heterogeneous expression of cell surface (FIG. 14A-B) and total cell ART1 (FIG. 14A, FIG. 19A). Both tumor cell lines had higher ratios of cell surface/total cell expression than did BEAS2B cells (59.5% and 55.4% vs. 29.2% respectively, FIG. 14C). Analysis of ART1 gene expression by RT-qPCR in tumor and matched normal lung tissue from 40 patients with stage I-III lung adenocarcinoma showed significantly higher mean expression in the cancer samples, driven by a fraction of the tumors with markedly higher expression (FIG. 14D). Of note, the matched tumors also had significantly lower expression of glycosylphosphatidylinositol specific phospholipase D1 (GPLD1), the only well characterized mammalian phospholipase regulating cleavage of GPI anchors (FIG. 19B). Cell-associated GPLD1 can release GPI-anchored proteins from the cell surface, but expression has been shown to be down-regulated with stress in lung cancer cells, suggesting that tumor cells are more likely to retain ART1 on the cell surface than benign cells.

To determine ART1 protein expression, a tissue microarray (TMA) of 493 stage I lung adenocarcinomas was analyzed for ART1 expression by immunohistochemistry. Staining for ART1 in the cancer cells was strong, moderate, and weak in 55%, 42% and 3% of the tumors, respectively (FIG. 19C and Table 2).

For the most part, ART1 expression by IHC in the cancer cells was diffuse cytoplasmic but staining concentrated near the cell periphery and membrane (membranous) was identified in 10% of the tumors (FIG. 14E and Table 3). Tumors with a mucinous histologic subtype, a rare tumor which only comprised 3.7% of the cohort, were particularly likely to express membranous ART1 compared to other histologic subtypes (44% vs. 8.4%). Tumors were also scored for infiltration of CD3, CD8, CD4 and FoxP3 T cells, CD20 (B cells), CD56 (natural killer (NK) cells) and CD68 or CD163 (macrophages) (Table 4). There was no correlation between overall ART1 staining intensity and immune cell infiltration (Table 2). However, tumors with membranous ART1 staining were significantly more likely to have low CD8 T cell infiltration as compared to tumors with only diffuse cytoplasmic ART1 (72% versus 44%, FIG. 19F, Table 3).

Next, transcriptomic data from a lung adenocarcinoma cohort (TCGA, PanCancer Atlas) was analyzed using the cBioportal platform to assess whether ART1 tumor expression was associated with differential expression of genes associated with CD8 T cell cytotoxicity; IFNγ (IFNG), Granzyme A (GZMA), Granzyme B (GZMB), Perforin 1 (PRF1) 41BB (TNFRSF9) as well as genes associated with immunoregulation; CTLA-4 (CTLA4), PD-1 (PDCD1), Tim-3 (HAVCR2), Lag-3 (LAG3) and Tigit (TIGIT). A heatmap was generated using cBioportals OncoPrint with clustering, which showed that high ART1 mRNA expression was associated with low expression of CD8 T cell cytotoxicity genes and immunoregulatory genes (FIG. 14G). Analysis of individual genes showed that patients with ART1^{high} tumors had significantly reduced mRNA expression of IFNG, GZMA, TNFRSF9, CTLA4, PDCD1 and TIGIT compared with patients with ART1^{low} tumors (FIG. 19D).

### ART1 Tumor Expression Exerts Immune Resistance in Mouse Lung Tumor Models

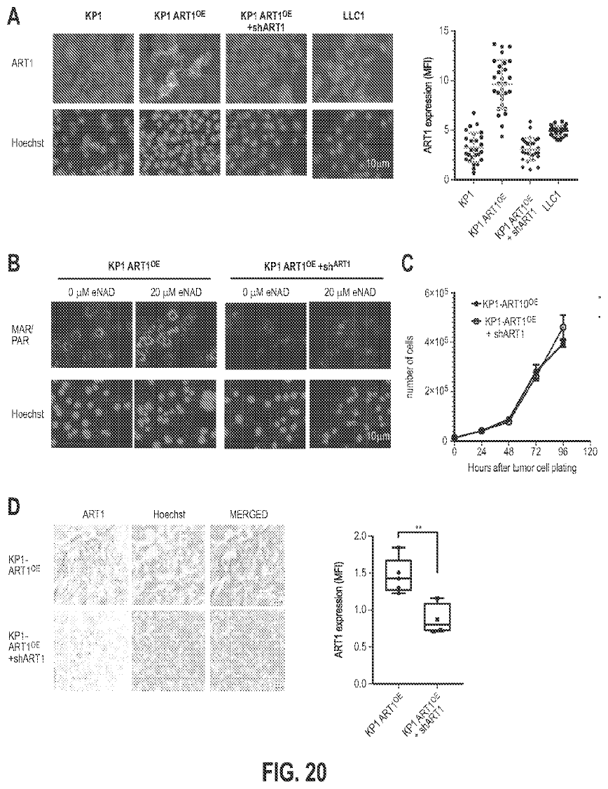

To test the hypothesis that ART1 expression protects tumors from T cell-mediated rejection, a mouse model of ART1 over-expressing NSCLC was developed. An ART1 plasmid was introduced into a KP1 cells, which were originally derived from inducible KRAS^{G12D/+}/p53^{-/-} mice (KP1-ART1^{OE}). The parent wild type KP1 line has low level ART1 cell surface expression at baseline, while the engineered KP1-ART1^{OE} line showed an approximately 9-fold increase in ART1 surface expression per cell by IF (FIG. 20A). In order to modulate ART1 expression, KP1-ART1^{OE} cells were transduced with a doxycycline-inducible short hairpin RNA (shRNA) targeting ART1 (shART1). Doxycycline-induced ART1-knockdown markedly reduced both ART1 cell surface expression (FIG. 20A) and ADP-ribosylation of tumor cell surface targets on the cancer cells themselves (FIG. 20B). Proliferation of KP1-ART1^{OE} cells remained unaffected by ART1 knockdown (FIG. 20C).

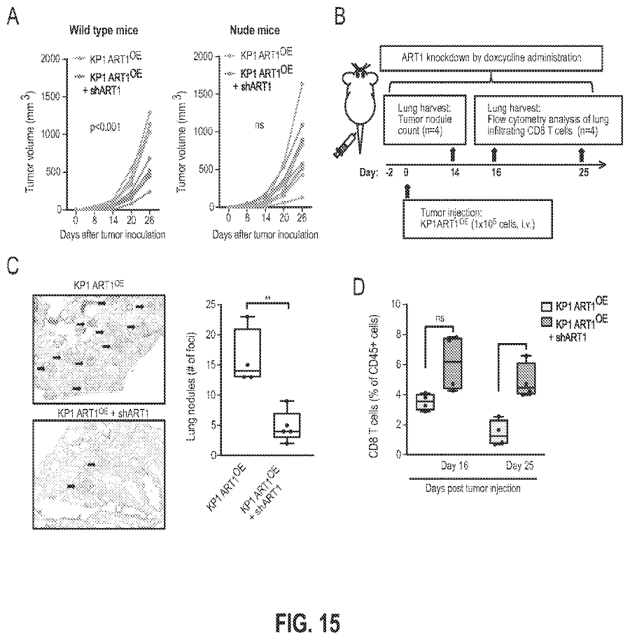

To test the effect of ART1 expression on tumor growth in vivo, KP1-ART1^{OE} cells were subcutaneously inoculated in immunocompetent wild-type and T cell-deficient nude C57BL/6 mice. Half of the mice in each group were given doxycycline to induce ART1 knockdown in vivo, which was confirmed by immunofluorescence staining of tumor specimens (FIG. 20D). In immunocompetent mice, KP1-ART1^{OE} flank tumors grew rapidly while doxycycline-induced ART1 knockdown significantly delayed flank tumor growth (FIG. 15A, left panel). In T cell-deficient nude mice, KP1-ART1^{OE} flank tumors had a similar growth rate as in wild type mice. However, the effect of ART1 knockdown on tumor growth was abrogated only in immunocompetent mice suggesting that the tumor-promoting effects of ART1 might be T cell-dependent (FIG. 15A, right panel). Next, the role of ART1 overexpression in an orthotopic lung tumor model was investigated.

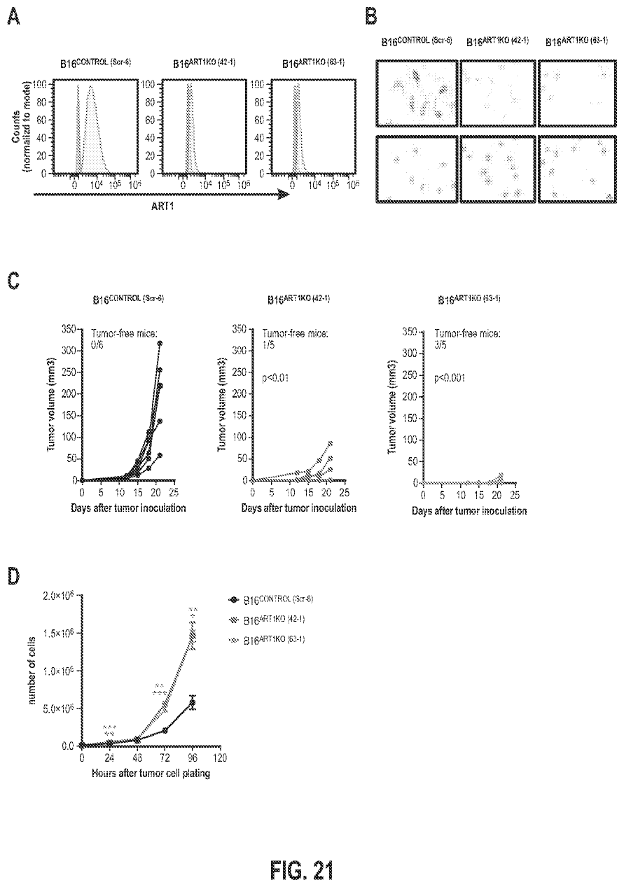

To generate KP1-ART1^{OE} lung tumors, KP1-ART1^{OE} cells were injected in the tail vein, and cohorts of the mice were given doxycycline to induce ART1 knockdown in vivo (FIG. 15B). ART1 knockdown resulted in a significantly decreased lung tumor burden at day 14, assessed by nodule frequency count from hematoxylin and eosin (H&E) stained lung sections (FIG. 15C). Lung CD8 T cell infiltration was determined by flow cytometry at days 16 and 25. At day 25 after tumor injection, mice with induced ART1 knockdown had significantly higher frequency of CD8 T cells among total lung tumor-infiltrating leukocytes (CD45+ cells) than mice bearing ART1-expressing tumors (FIG. 15D). The percentage of lung tumor-infiltrating CD8 T cells was decreased at day 25 compared to day 16 in control mice, consistent with a loss of immune control associated with tumor progression. To test the role of ART1 in tumor progression in a second immunocompetent mouse tumor model, a melanoma line was chosen, as human melanomas are shown to strongly express ART1 in the Human Protein Atlas. B16-F10 mouse melanoma cells have high intrinsic ART1 cell surface expression and an ART1-negative derivative was generated using CRISPR/Cas9 and two different guide RNA (FIG. 21A-B). We observed that subcutaneous injection of ART1-expressing B16-F10 cells in syngeneic immune competent mice resulted in formation of rapidly growing flank tumors, while ART1-deficient B16-F10 cells showed markedly impaired tumor growth or failed to form palpable tumors (FIG. 21C). The impaired growth of ART1-deficient B16-F10 cells in vivo was not due to decreased fitness of the cancer cells themselves since, in vitro, ART1-deficient cells proliferated faster than ART1-expressing B16-F10 cells (FIG. 21D).

### ART1 Blockade Reduces Lung Tumor Burden and Promotes Infiltration of P2X7R⁺ CD8 T Cells

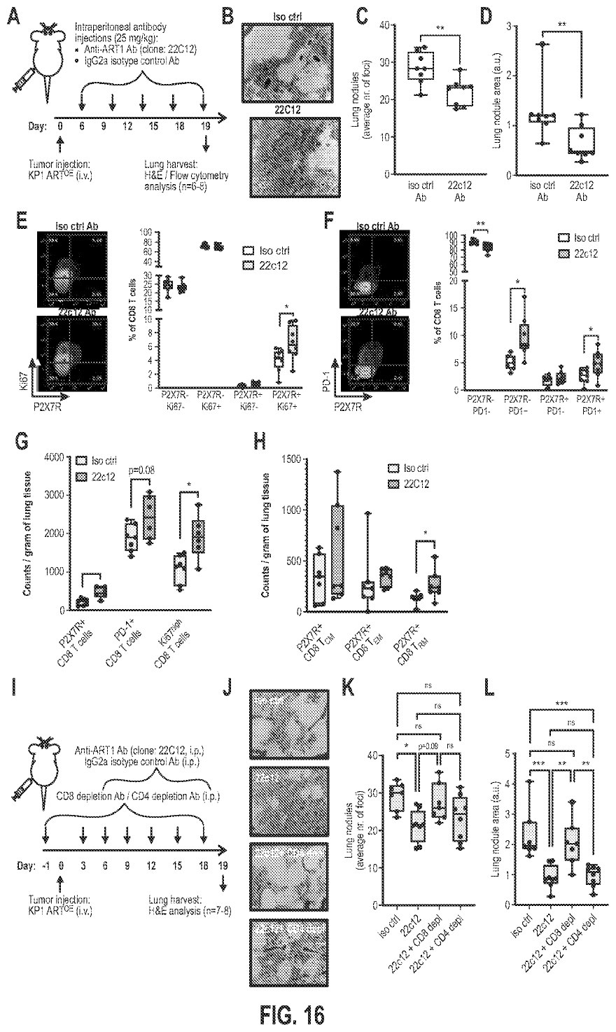

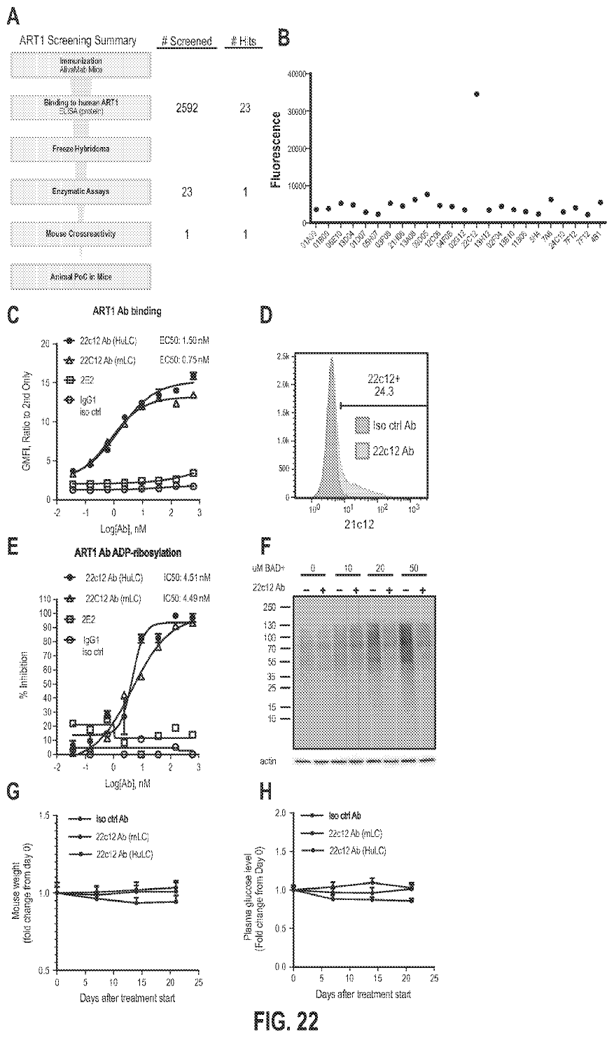

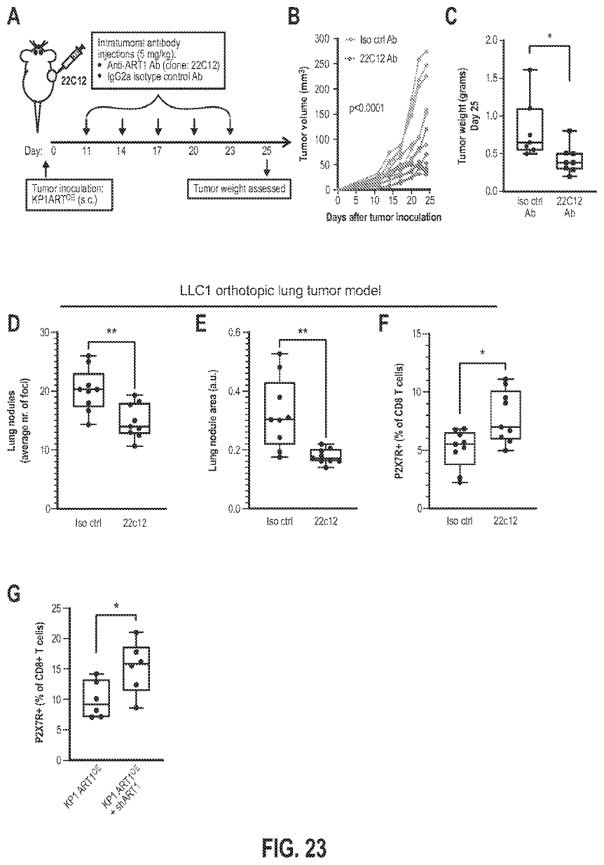

Therapeutic targeting surface ART1 was investigated using a 22C12 monoclonal antibody targeting ART1. Therapeutic antibody candidates were initially developed through immunization of AlivaMab® Mouse transgenic mice with a human immunoglobulin repertoire utilizing human ART1. Candidate antibodies that bound to both human and mouse ART1 and inhibited mono-ADP-ribosylation were further developed (FIG. 22A-B). The lead candidate, 22C12, which potently inhibited ART1 enzymatic activity in the primary screening assay, was further developed. 22C12 antibody clones with mouse light chains (22C12 (mLC)) and human light chains (22C12 (HuLC)) were generated which were tested for activity in vitro and in vivo. Binding of 22C12 antibodies to HEK 293 cells transfected with ART1 (HEK-ART1^{OE}) was determined by NAD-Glo assay showing half-maximum binding (EC₅₀) values in the range of 0.8-1.5 nM (FIG. 22C). Binding of 22C12 to KP1-ART1^{OE} cells was assessed by flow cytometry staining (FIG. 22D). Half-maximum inhibition of ADP-ribosylation (IC₅₀), by 22C12 antibodies, as determined by cell surface ADP-ribosylation of HEK-ART1^{OE} cells, was achieved at 4.5 nM antibody concentration (FIG. 22E). The ability of 22C12 Ab to block cancer cell induced mono-ADP-ribosylation was confirmed in KP1-ART1^{OE} cells co-cultured with NAD⁺ (FIG. 22F). We assessed potential toxicity of systemic administration of the 22C12 antibodies in tumor-naïve mice by i.p. injections of 25 mg/kg every three days for three weeks and monitored for weight loss and blood glucose levels at baseline and every week until the end of the study. The mice remained normal in appearance, activity, gait and alertness throughout the study (FIG. 22G-H). To test the in vivo anti-tumor activity of 22C12, intratumoral injections of the 22C12 antibody or an isotype subclass-matched control antibody (5 mg/kg) were performed on subcutaneously implanted KP1-ART1^{OE} flank tumors starting when tumors had become palpable (day 11). The injections were repeated every three days until day 23. On day 25, mice were sacrificed, and tumors were weighed (FIG. 23A-C). ART1 blockade resulted in a significantly delayed tumor growth compared to tumors treated with isotype control antibody (FIG. 23B), with average tumor weight at day 25 significantly lower in the mice treated with 22C12 compared with isotype control antibody (FIG. 23C). Next, a study was designed to assess the anti-tumor effect and immunomodulatory properties of ART1 blockade in the orthotopic KP1-ART1^{OE} lung tumor model. Mice were treated intraperitoneally with 22C12 antibody (25 mg/kg) or the equivalent dose of isotype control antibody starting on day 6 after tumor injection until day 18 (FIG. 16A). On day 19, mouse lungs were fixed and stained with H&E to assess lung tumor burden, which showed fewer and significantly smaller tumor nodules in mice treated with 22C12 antibody compared with isotype-treated mice (FIG. 16B-D). To confirm the observations in a second lung tumor model endogenous surface ART1 expression on Lewis lung carcinoma (LLC1) cells was assessed (FIG. 20A). In an LLC1 orthotopic lung tumor model (treatment strategy as in FIG. 16A), mice treated with ART1 blockade had reduced lung tumor burden compared to control mice (FIG. 23D-E).

Next, flow cytometry analysis was performed on digested KP1-ART1^{OE} tumor-bearing lungs to assess how ART1 blockade affects P2X7R expression in the CD8 T cell compartment. Ki67 expression was assessed indicating the state of proliferation and expression of the immunoregulatory receptor PD-1 indicating activation and tumor-engagement. Considering recent studies showing a susceptibility of P2X7R+ T_{RM} to NICD, it was assessed whether ART1 blockade would increase the infiltration of P2X7R+ memory CD8 T cell subsets including central memory (T_{CM}, CD62L+CD44+CD69−) Effector memory (T_{EM}, CD62L-CD44+CD69−) and T_{RM} (CD62L−CD44+CD69+) CD8 T cells.

The majority of P2X7R+ CD8 T cells co-expressed Ki67, indicating that they were in a proliferative state, and it was observed that ART1-blockade increased the percentage of P2X7R+Ki67+ but not of the P2X7R−Ki67− CD8 T cell subset (FIG. 16E). PD-1 was co-expressed on a subset of P2X7R+ CD8 T cells and we observed an increase in the percentage of PD-1 expression both in the P2X7R− and P2X7R+ CD8 T cell subset following ART1 blockade (FIG. 16F). Further, it was found that absolute numbers of CD8 T cells expressing P2X7R and Ki67 normalized to lung tissue weight were elevated in mice treated with ART1 blockade compared to control mice (FIG. 16G). ART1 blockade increased the infiltration of P2X7R+ T_{RM} while P2X7R+ T_{EM} and T_{CM} populations were not significantly increased (FIG. 16H). Enrichment of P2X7R+ CD8 T cells, was observed following ART1 blockade in the LLC1 lung tumor model (FIG. 23F) and following ART1 knockdown in the KP1-ART1^{OE} orthotopic lung tumor model (FIG. 23G).

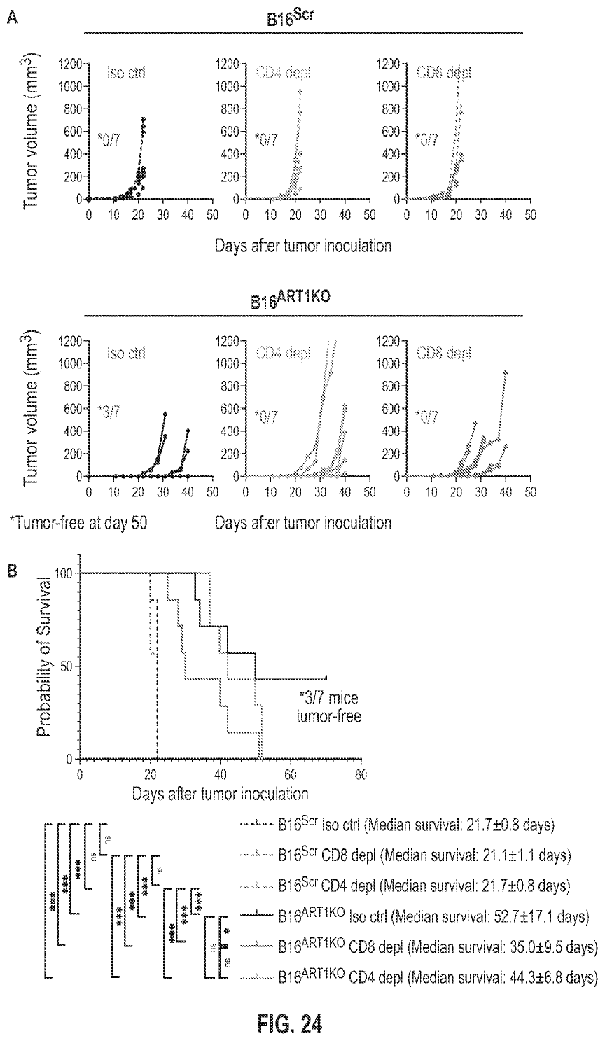

These findings indicated that tumor ART1 expression modulates intratumoral CD8 T cells. Hence, we next investigated whether tumor resistance exerted by ART1 expression was dependent on suppression of CD8 T cell-mediated immunity. To this end, the KP1ART1^{OE} orthotopic lung tumor model was employed, where CD8 and CD4 T cells were depleted in the mice by administration of monoclonal antibodies. ART1 was blocked by i.p. injection of 22C12 starting on day 6 after tumor injection (FIG. 16I). Lungs were harvested on day 19 after tumor inoculation to assess tumor burden by H&E staining. In our quantification of lung nodules, we observed a reduced number of tumor nodules in the mice treated with ART1 blockade compared to control mice while the was no significant differences between mice treated with ART1 blockade and CD8- or CD4 T cell depleting antibodies (FIG. 16J-K). However, analysis of the average area of the tumor nodules showed that CD8 depletion abrogated the reduction in tumor nodule size observed with ART1 blockade alone while CD4 T cell depletion did not significantly change the anti-tumor effect induced by ART1 blockade (FIG. 16J, L). To confirm whether the dependency of ART1-mediated anti-tumor effect on CD8 T cells was applicable across different tumor models, the CD8/CD4 depletion study was performed in the B16-F10 ART1 knockout model (FIG. 24). It was observed that subcutaneous injection of ART1-expressing B16-F10 cells (B16^{CONTROL}) in syngeneic immune competent mice resulted in formation of rapidly growing flank tumors. In mice bearing B16^{CONTROL} tumors, CD8- and CD4-depletion resulted in similar tumor progression as in mice receiving isotype control antibody (FIG. 24A, upper panel, 24B). Mice inoculated with ART1-deficient B16-F10 (B16^{ART1KO}) cells showed markedly impaired tumor growth or failed to form palpable tumors (3/7 mice tumor free at day 70). In mice bearing B16^{ART1KO} tumors, CD8 T cell-depletion resulted in higher tumor burden and significantly reduced survival compared to mice bearing B16^{ART1KO} tumors treated with isotype control antibody. Mice that received CD4 T cell depletion developed tumors albeit with slower tumor growth compared with CD8 depleted mice and no significant different in survival compared with isotype control treated animals (FIG. 24A, lower panel, 24B).

P2X7R Expression on CD8 T Cells Renders them Susceptible to ART1-Mediated NICD

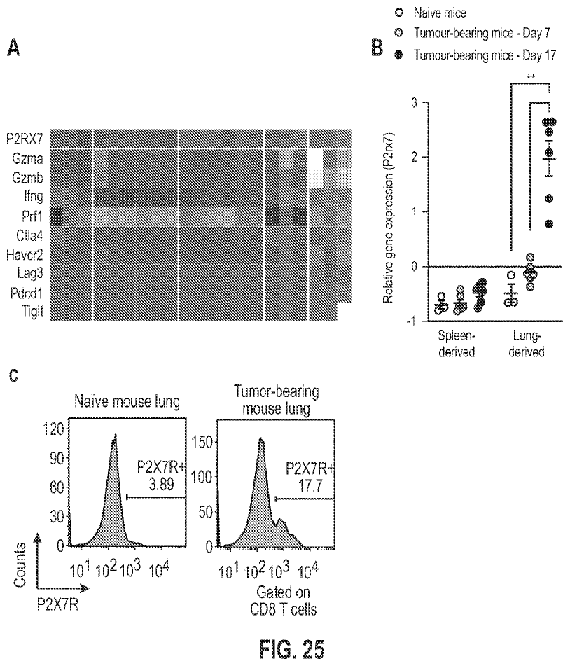

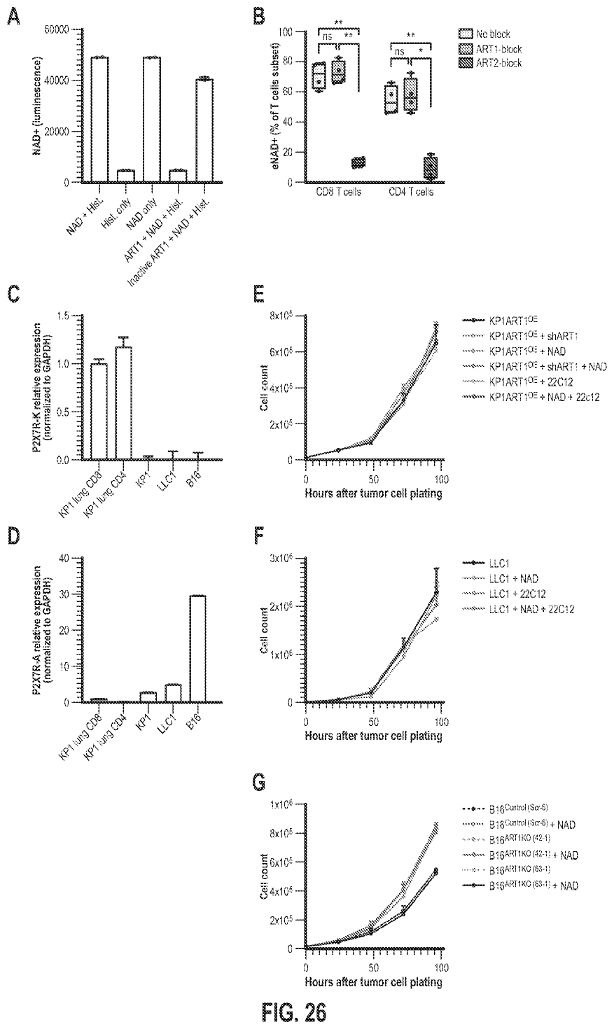

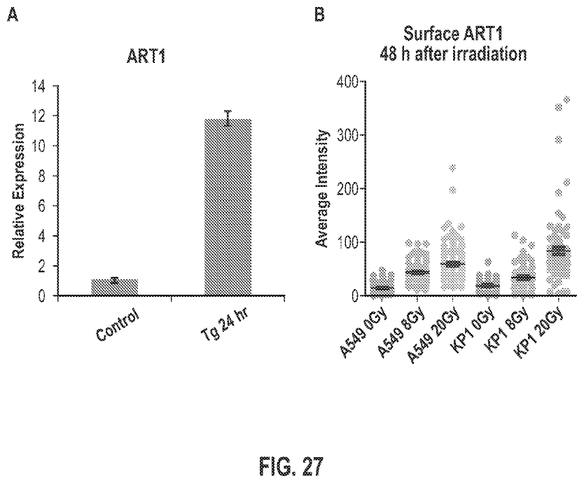

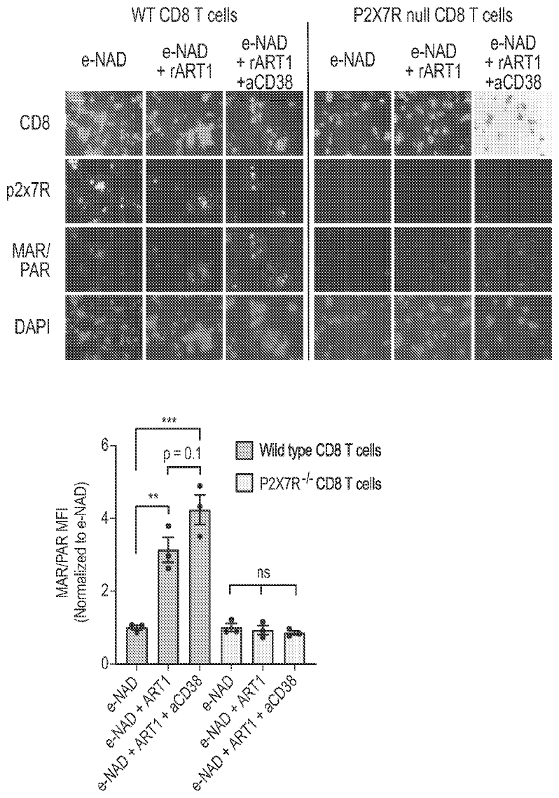

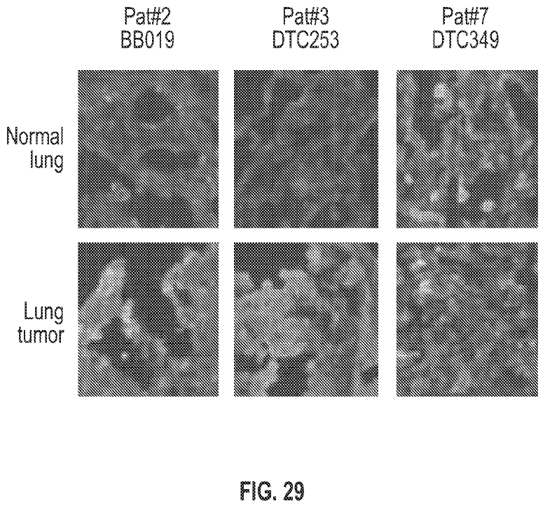

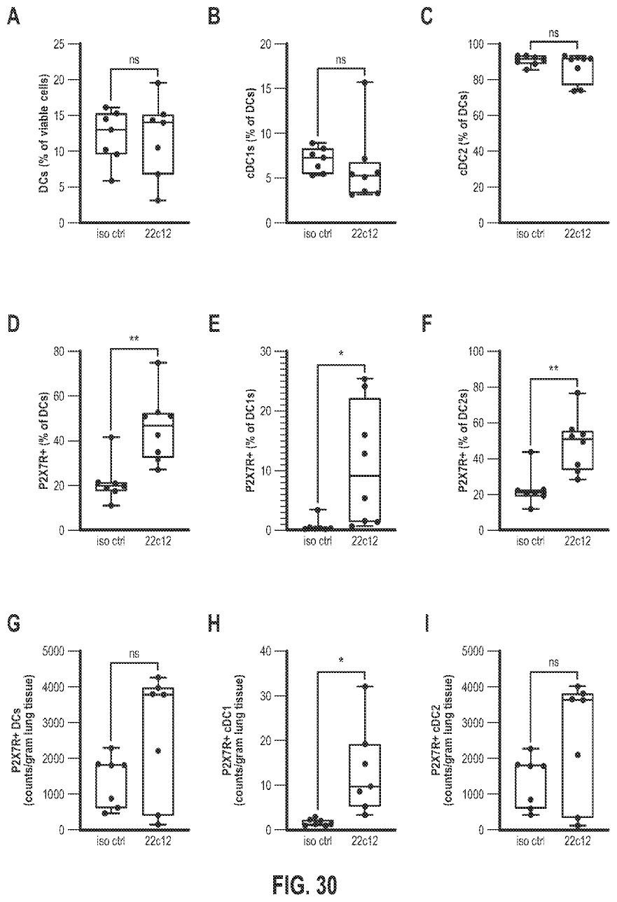

In acutely inflamed tissues characterized by increased extracellular NAD⁺, CD8 T cells expressing P2X7R are eliminated by NICD. However, the importance of CD8 T cell P2X7R expression for anti-tumor immunity in lung cancer has not been well described. It was assessed whether P2RX7 expression was altered in murine CD8 T cells over the course of lung tumor progression. To this end, RNA sequencing analysis was performed on CD8 T cells isolated from lungs and spleens from mice orthotopically inoculated with wild type KP1 cells, which express low levels of ART1. Gene expression of P2RX7 as well as genes associated with CD8 T cell cytotoxicity and immunoregulation was assessed in CD8 T cells isolated on day 7 and 17 after tumor inoculation as well as in CD8 T cells from naïve non-tumor bearing mouse lungs. P2RX7 expression, as well as IFNG, PRF1, PDCD1, CTLA4, HAVCR2, LAG3, and TIGIT was moderately increased in CD8 T cells isolated from mouse lungs with KP1 tumor burden at day 7 after KP1 injection and markedly increased at day 17 compared with CD8 T cells isolated from lungs of naïve mice (FIG. 25A-B). The observed changes in P2RX7 expression appeared to be confined to the lung-resident CD8 T cells, as we did not observe similar expression changes in spleen-derived CD8 T cells (FIG. 26). Increased surface expression of P2X7R in populations of CD8 T cells from wild type KP1 tumor-bearing mice was confirmed by flow cytometry (FIG. 25C).

In light of the findings that P2X7R+ CD8 T cells were enriched in lung tumor tissues following genetic or pharmacological inhibition of ART1, it was assessed whether P2X7R is a target for ART1-mediated ADP-ribosylation and NICD of lung-tumor infiltrating T cells. To this end, an in vitro co-culture assay was established where T cells isolated from lungs of wild type KP1 tumor-bearing mice were incubated with or without recombinant murine ART1 (rART1). It was confirmed that T cells do not express ART1 by qPCR and the enzymatic activity of rART1 was confirmed by NAD-Glo assay. Etheno-tagged NAD⁺ (eNAD) was added to the co-cultures which was detectable by flow cytometry to identify ADP-ribosylated cells, while DAPI (4′,6-diamidino-2-phenylindole) staining was used to measure cell death. An ART2-blocking nanobody (s+16a), was used to block ART2, which is expressed on murine lymphocytes and can mediate auto-ADP-ribosylation of T cells in cis. Surface ART1 was blocked using the 22C12 monoclonal antibody. The ability of the ART2 and ART1 blocking antibodies to inhibit ADP-ribosylation was confirmed in an experiment where T cells were cultured in the presence of eNAD alone. ART2 blockade resulted in reduced ADP-ribosylation of CD8 T cells from 70.1±8.8% to 12.9±2.6% and of CD4 T cells from 54.6±9.6% to 9.3±7.2%. ART1-blockade did not affect ADP-ribosylation of CD8 or CD4 T cells The CD38 blocking antibody (NIMR-5) was used to assess whether expression of CD38 could play a cytoprotective role by catabolizing free NAD⁺ from the immediate micromilieu.

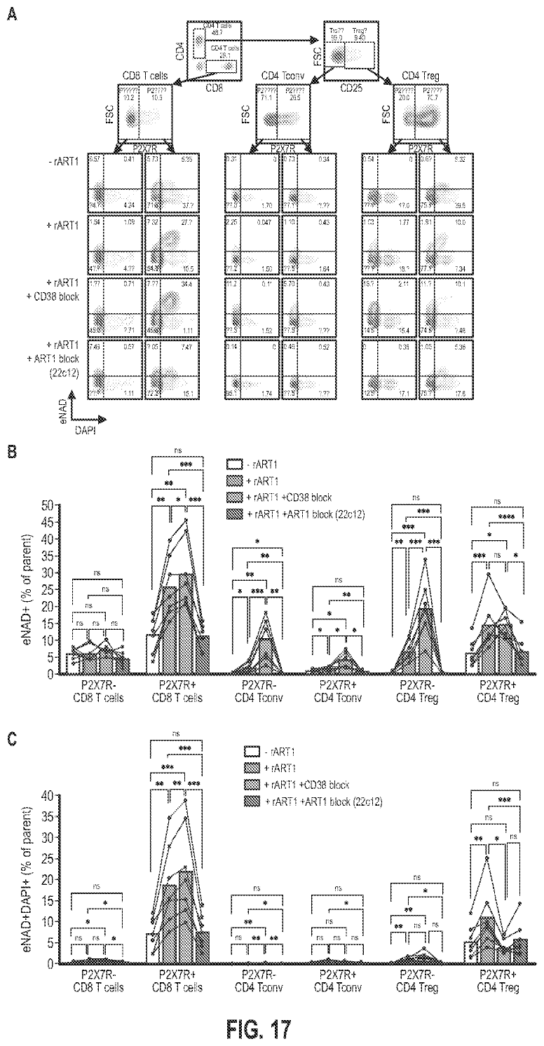

Considering previous studies which show a susceptibility of CD4+ regulatory T cells (CD4 Tregs) to NICD via P2X7R (22), ADP-ribosylation and NICD were separately analyzed on CD4 Tregs, CD4 Tconv cells and CD8 T cells as well as on P2X7R+ and P2X7R-fractions of the T cell subsets separately (FIG. 17). Average P2X7R expression was 9.3±2.4% of CD8 T cells, 21.6±3.9% of CD4 Tconv and 80.8±2.6% of CD4 Tregs.

Total ADP-ribosylation represented by positive eNAD staining (FIG. 17B) was assessed. Co-culture with rART1 did not significantly increase ADP-ribosylation of either P2X7R− or P2X7R+ CD4 Tconv cell subsets while both P2X7R− and P2X7R+ CD4 Tregs were significantly more ADP-ribosylated in the presence of rART1. CD38 blockade significantly increased ADP-ribosylation of both P2X7R− and P2X7R+ CD4 Tconv and CD4 Tregs in the presence of rART1. P2X7R+ but not P2X7R− CD8 T cells were sensitive to ART1-mediated ADP-ribosylation which was increased in the presence of CD8 blocking antibodies. Addition of ART1 blocking antibody (22c12) to the co-culture reduced ART1-mediated ADP-ribosylation of CD8 T cells as well as CD4 Tconv and CD4 Tregs to baseline levels (FIG. 17B). NICD was measured which was determined by the frequency of cells that stained positive for both eNAD and DAPI (FIG. 17C). P2X7R+ CD8 T cells, but not P2X7R-CD8 T cells, were susceptible to ART1-mediated NICD, which was exacerbated by CD38 blockade. ART1-blockade reduced NICD of P2X7R+ CD8 T cells to baseline levels. While we detected low NICD levels of CD4 Tconv cells, NICD of CD4 Tregs was significantly elevated in the presence of rART1, primarily in the P2X7R+ subset and was reduced to baseline levels upon ART1-blockade. In contrast to its effect on P2X7R+ CD8 T cells, CD38 blockade reduced ART1-mediated NICD of P2X7R+ CD4 Tregs (FIG. 17C).

Together, the data indicate that CD8 T cells and CD4 Tregs are susceptible to ART1-mediated ADP-ribosylation and NICD via the P2X7R. The significant ADP-ribosylation of P2X7R-CD4 Tregs, and to a lesser extent P2X7R− and P2X7R+ CD4 Tconv cells, following co-culture with rART1 and CD38 blockade indicate that additional targets exist on these cells, which are sensitive to ADP-ribosylation by ART1 in the absence of CD38 expression. However, the cytoprotective role of CD38 against ART1-mediated NICD observed in P2X7R+ CD8 T cells was reversed in P2X7R+ Tregs.

It was hypothesized that the difference in susceptibility of CD8 T cells and CD4 Tconv cells to ART1-mediated NICD could be explained by a difference in relative expression of the P2RX7 splice variants P2RX7-a and P2RX7-k, the latter of which has been shown to be more prone to trigger NICD when mono-ADP-ribosylated compared with the P2RX7-a variant. To this end, RNA from CD8 T cells and CD4 Tconv cells isolated from KP1 tumour-bearing mouse lungs was analysed by qPCR for expression of P2RX7-a and P2RX7-k. As suggested by previous studies tumor cells are also known to express P2X7R in addition to immune cells which could render them susceptible to ART1-mediated NICD. Therefore, RNA isolated from KP1, B16 and LLC1 mouse tumor cells was analyzed for expression of the P2RX7 splice variants. It was observed that CD8 T cells CD4 Tconv cells isolated from KP1 tumor-bearing lungs expressed comparable levels of P2RX7-k while all tumor cells expressed low levels of P2RX7-k (FIG. 26C). P2RX7-a expression was low in both CD8 T cells and CD4 Tconv cells while we detected expression in KP1 and LLC1 cells and highly expressed in B16 cells, which may protect ART1-expressing tumor cells from NICD following auto-ADP-ribosylation (FIG. 26D). In line with these findings, proliferation assays demonstrate that ART1-expressing tumor cells grown in the presence of NAD+ and/or ART1 blockade have no significant differences in cell growth (FIG. 26E-G).

ART1 Expressing Human Lung Tumors have Reduced Infiltration of P2X7R+CD8 T Cells

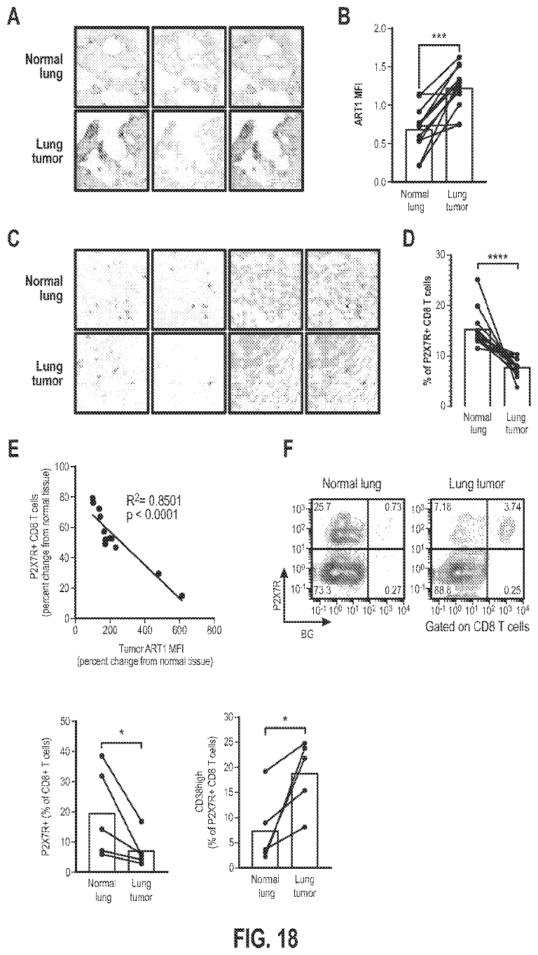

It was determined whether infiltration of P2X7R⁺ CD8 T cells is modulated in human lung tumors with confirmed expression of ART1. Twelve matched lung adenocarcinomas and adjacent normal lung tissue specimens were stained for ART1 expression by immunofluorescence. A heterogeneous expression of membranous ART1 staining was observed in both normal and cancerous lung tissue. The patient samples were assessed for infiltration of P2X7R+ CD8 T cells by immunofluorescence staining, which revealed that the percentage of P2X7R+ CD8 T cells among total CD8 T cells was significantly lower in lung tumor tissue compared with normal lung tissue (FIG. 18C-D). A linear regression analysis of the percent change in P2X7R positivity among CD8 T cells between normal lung and tumor tissue and the percent change of ART1 MFI between normal lung and tumor tissue revealed a clear inverse correlation between P2X7R+ CD8 T cells and ART1 MFI (Pearson correlation, R²=0.85, p<0.0001) (FIG. 18E). Further, flow cytometry analysis was performed on dissociated tissue from five lung adenocarcinoma patients to assess CD8 T cells and their expression of P2X7R and CD38. The percentage of P2X7R⁺ CD8 T cells among total CD8 T cells was lower in the tumor compared to adjacent lung tissue (7.1±5.6% versus 19.5±14.8%, p<0.05) (FIG. 18F-G). Tumor-infiltrating P2X7R⁺CD8 T cells expressed high levels of CD38 which was significantly higher compared to normal lung tissue (FIGS. 18F and H). These data indicate that expression of ART1 in human lung cancer, similarly to the mouse models, is associated with decreased tumor infiltration by P2X7R⁺ CD8 T cells, whose co-expression of CD38 may be necessary to avoid ART1-mediated NICD in the tumor microenvironment.

### DISCUSSION

The tumor immune contexture is associated with prognosis and response to immunotherapy, with CD8 T cell infiltration generally serving as an indicator of an ongoing anti-tumor immune response which can be reinvigorated by ICI. To improve patient outcomes, it is critical to gain an improved understanding of the factors that regulate CD8 T cell infiltration and their function in the tumor. In normal tissue, ART-mediated ADP-ribosylation and NICD regulate T cell homeostasis following tissue damage or infection. However, whether this mechanism is involved in the regulation of CD8 T cell infiltration within tumors, and whether expression of ARTs is dysregulated in human cancer, has not been previously investigated. In the present study, ART1 overexpression on human lung cancer cells was shown to be associated with poor survival and reduced intratumoral CD8 T cells, specifically a reduction in the P2X7R⁺ CD8 T cell subset. Furthermore, expression of ART1 in mouse tumors promoted tumor growth in immune competent but not in T-cell deficient mice or following CD8 T cell depletion and is associated with a reduction in tumor-infiltrating P2X7R⁺CD8 T cells. In vitro, P2X7R⁺ but not P2X7R⁻ CD8 T cells were susceptible to ART1-mediated ADP-ribosylation and to NICD which was exacerbated upon blockade of CD38 indicating a potential cytoprotective role. Overall, these data identify ART1 expression in lung cancer, and possibly in other cancers, as a novel regulator of CD8 T cell infiltration in the tumor microenvironment. ART1 is an actionable target to improve immune-mediated tumor control. As an extracellular, membrane-anchored enzyme, ART1 should be highly druggable. Here, it was demonstrate that treatment with a monoclonal antibody that binds to and inhibits ART1-induced ADP-ribosylation had therapeutic benefits in preclinical models, resulting in reduced growth of ART1⁺ lung cancer and increased tumor-infiltration of activated and proliferating P2X7R⁺ CD8 T cells.

Despite an expanding knowledge of the role of mono-ADP-ribosylation in tumor development, ART1 has only recently been described to play a role in cancer progression. In a model of mouse colon adenocarcinoma, Xu et al. demonstrated that overexpression of ART1 facilitated tumor growth, while knockdown inhibited tumor growth in various immune competent models. This effect was attributed to cis-ADP-ribosylation of integrin and Rho effector family members, subsequently affecting downstream mediators of cellular migration. The anti-tumor effects of ART1 knockdown or blockade in mouse lung cancer models is dependent on CD8 T cells. In vitro, knockdown of ART1 had no effect on KP1-ART1^{OE} cell proliferation and actually enhanced tumor cell proliferation of B16 mouse melanoma cells yet resulted in impaired tumor growth in vivo in immunocompetent mice. Thus, although ART1 has cancer cell-intrinsic effects that may be model-dependent, the immune suppressive effects of ART1 expression seem to dominate in the present in vivo models.

These effects are predominantly mediated through mono-ADP-ribosylation of P2X7R on CD8 T cells. Tumor expression of P2X7R is linked to improved survival in NSCLC patients, although it remains unclear whether P2X7R expression on CD8 T cells is associated with a survival advantage. Preclinical studies have painted a complex picture of P2X7R role in tumor progression and anti-tumor immunity. Di Virgilio and colleagues demonstrated increased tumor progression associated with low CD8 T cell infiltration in P2X7R− deficient mice in the B16-F10 melanoma model. In contrast, administration of a P2X7R antagonist to wild type tumor-bearing mice resulted in reduced tumor growth and increased immune activation. These seemingly contradictory results can be explained by the fact that P2X7R is required for the activation of the inflammasome in dendritic cells by ATP released by dying cancer cells, which was required for priming of anti-tumor CD8 T cells. Thus, in P2X7R-deficient mice, there is a failure to initiate the anti-tumor immune response. In contrast, pharmacological inhibition of P2X7R after CD8 T cell priming has already occurred may prevent NICD of P2X7R⁺ CD8 T cells induced by ART1, which is highly expressed by B16 melanoma cells.

Herein it was demonstrated that blocking ART1 in established tumors promotes P2X7R⁺ CD8 T cell infiltration.

The mechanistic studies showed that P2X7R+ CD8 T cells, and to a lesser extent CD4 Tregs, are susceptible to ART1-mediated NICD via the P2X7R. The lack of susceptibility of P2X7R+ CD4 Tconv cells to ART1-mediated NICD is intriguing. It was hypothesized that selective expression of the P2X7R-a splice variant, which is less prone to trigger NICD, would explain this disparity but we did not detect high levels of this variant in either CD4 Tconv or CD8 T cells isolated from tumor-bearing lungs indicating that there may be other cell-intrinsic mechanisms at work that renders P2X7R+ CD8 T cells uniquely sensitive to ART1-mediated NICD. However, the finding that tumor cells express high levels of P2X7R-A may indicate a mechanism by which tumor cells can express ART1 and P2X7R simultaneously without triggering auto-ADP-ribosylation and NICD.

Recent studies have shown that P2X7R expression in recirculating memory CD8 T cells is essential for extracellular ATP-driven maintenance of mitochondrial function and metabolic fitness, that generation of CD8 T_{RMs} via TGFβ sensing is dependent on P2X7R and that T_{RM} homeostasis is regulated by NICD via the P2X7R. In line with these findings, ART1 was found to block increases infiltration of P2X7R+ CD8 T_{RMs} in tumor-bearing lungs Together with the observation that lung tumor-infiltrating CD8 T cells have elevated expression of P2X7R and co-express cytotoxic and immunoregulatory markers, it is likely that P2X7R+CD8 T cells infiltrating lung tumors represent a critical tissue-resident subset of memory T cells with anti-tumor activity and that they are targeted by ART1 tumor expression.

T cell expression of CD38 is also likely to be a critical component in determining whether cells undergo NICD. CD38 is upregulated on mouse and human T cells upon activation and differentiation and may represent a cytoprotective mechanism to avoid ADP-ribosylation and NICD in NAD⁺ enriched inflamed tissues. The present results indicate that P2X7R⁺ CD8 T cell,s and P2X7R+ CD4 Tregs to a lesser extent, are susceptible to ART1-mediated ADP-ribosylation and NICD, and that CD38-blockade enhances ADP-ribosylation and NICD of P2X7R+ CD8 T cells in presence of ART1. In addition, it was found that a subset of CD38⁺ P2X7R⁺ CD8 T cells were enriched in ART1⁺ human lung tumors, suggesting that CD38 expression may enable survival of a subset of anti-tumor CD8 T cells that would have otherwise been eliminated by ART1-mediated NICD via the P2X7R. Importantly, the in vitro experiments show that ART1-mediated NICD of CD4 Tregs was abrogated following CD38 blockade. Hence, in a clinical setting, treatment of ART1-positive adenocarcinoma patients with CD38 blockade could have the dual effect of exacerbating NICD of CD8 T cells while protecting CD4 Tregs, thus skewing the CD8 T cell to CD4 Treg ratio which is associated with immunotherapy response and tumor rejection.

These findings implications for the design of clinical studies targeting CD38 to enhance anti-tumor immunity. In addition to T cells, CD38 is expressed by other immune cells and some cancer cells and has been shown in pre-clinical studies to contribute to acquired resistance to PD-1/PD-L1 blockade by converting NAD⁺ into ADPR, a precursor of adenosine, which has broad immune suppressive function. The anti-CD38 antibody daratumumab was recently tested in combination with atezolizumab (an anti-PD-L1 antibody) in NSCLC patients in a clinical trial (NCT03023423). This study was terminated early because of increased mortality in the combination treatment arm. Although the reasons for this outcome are unclear, it is intriguing to consider whether increased NICD of anti-tumor T cells could have contributed. ART1 expression has been demonstrated to increase following cellular stress, so its expression in tumors may be highly dynamic and potentially fluctuate depending on the degree of inflammation in the TME as well as in response to treatment. Hence, the immunomodulatory effects of ART1 may play an even more significant role following treatment with cytotoxic agents such as chemotherapy and radiation. Such treatments will also contribute to increased levels of extracellular NAD⁺ following cell death, potentially priming the local microenvironment for ART1-induced mono-ADP-ribosylation. Thus, more studies into the role of ART1 as an actionable barrier to response to combinations of cytotoxic agents with immunotherapy are needed.

Thus, in NSCLC ART1-expressing tumor cells eliminate tumor-infiltrating CD8 T cells via NICD. The present findings suggest that ART1 tumor expression may have prognostic and predictive value in lung cancer patients undergoing immunotherapy. Pharmacologic targeting of ART1 may potentiate CD8 T cell-mediated immune responses in NSCLC patients.

### Materials and Methods

Statistical analysis: Human patient data: For the NSCLC TMA, continuous variables are reported as median (interquartile range [IQR]) and categorical variables are reported as count (percent). Chi-square or Fisher's exact tests were used to compare a categorical variable between independent groups. A Mann-Whitney U test was used to compare a continuous variable between two independent groups. Wilcoxon signed rank test was used to assess statistically significant differences in gene expression data from paired samples. Paired t-test was used to determine statistically significant differences in ART1 expression determined by immunofluorescence surface staining in paired samples. ART1 expression data were square root transformed, while percentages of tissue-infiltrating immune cells were log-transformed prior to statistical testing by paired t-test to ensure the underlying assumptions of the test were met. Statistically significant differences in ART1 MFI on human lung tumor cell lines was determined by one-way ANOVA with Tukey's test for multiple comparisons.

Mouse experiment data: Data consisting of counts, percentages and expression data were log-transformed or square root transformed where indicated prior to statistical testing by Welch's t-test. Tumor growth data comparing the effect of induced ART1-knockdown in KP1-ART1^{OE} tumors or ART1 knockout in B16-F10 tumors were analyzed by repeated-measures ANOVA with Geisser-Greenhouse correction. A mixed model analysis was used to determine statistically significant difference in tumor growth between mice treated with ART1-blocking antibody or isotype control antibody. Statistically significant differences in ART1 MFI on KP1 cells and KP1ART1^{OE} with or without shART1 induction was determined by one-way ANOVA with Tukey's test for multiple comparisons. All statistical tests were 2-sided and considered statistically significant with p<0.05. Data analysis was performed using SPSS software version 25 (IBM Corp.) or GraphPad Prism Version 8 (GraphPad).

Patient sample collection and analysis: Human lung adenocarcinoma samples for immunofluorescence and flow cytometry staining as well as RNA extraction and qPCR analysis were obtained from New York Presbyterian Hospital/Weill Cornell Medical College in accordance with a protocol approved by the IRB (IRB #1008011221). IHC staining for ART1 was performed on a TMA of 493 stage I lung adenocarcinomas (Suzuki et al., 2013) (Primary anti-human ART1 Ab: Santa Cruz, Catalog #sc-20255). The TMA was scored in a blinded fashion for intensity and location of ART1 staining. Intensity of ART staining were scored as (1) negative, (2) weak, (3) moderate or (4) strong. Location of staining was scored as (1) cytoplasmic, (2) membranous or (3) both cytoplasmic and membranous. In addition, TMA was scored for (1) low, (2) intermediate or (3) high infiltration of immune cell subsets; Pan T cells, CD4 T cells, CD8 T cells, Tregs, B cells and macrophages in tumor and stroma using markers; CD3, CD4, CD8, FoxP3, CD20, CD56, CD68 and CD163. NK cell infiltration in tumor and stroma was determined as absent or present using the CD56 marker. The scoring cell number cutoffs are described in Table 4.

Animals: All animal work was done following a protocol approved by the Institutional Animal Care and Use Committee of New York Presbyterian Hospital/Weill Cornell Medical College (IACUC #2010-0050, 2015-0028). Wild type C57BL/6 mice (strain: C57BL/6NTac) and athymic nude mice (strain: B6.Cg/NTac-Foxn1nu NE10) were purchased from Taconic Biosciences. All mice were maintained under pathogen-free conditions in the Weill Cornell Medicine animal facility.

Animal tumor models: For ART1 knockdown in vivo, Doxycycline was delivered to mice in drinking water containing sucrose (0.1 mg/mL doxycycline in 50 g/L sucrose) 48 to 72 hours before injection of KP1-ART1^{OE} cells. Control animals received water containing sucrose only. Water was changed every 4 days. Orthotopic lung tumor model: 0.5×10⁵ KP1-ART1^{OE} cells were resuspended in 100 μl PBS and injected into the tail vein of immunocompetent C57BL/6 mice (4-6 weeks old). For lung tumor burden evaluation at the indicated endpoints, mice were sacrificed, parts of the tumor-bearing lungs were formalin-fixed, paraffin-embedded and sectioned for subsequent H&E stain and blinded enumeration of lung nodules. The remaining parts of the tumor-bearing lungs were weighed and dissociated into a single cell suspension and stained for analyzed by flow cytometery for characterization of CD8 T cells. Ectopic flank tumor model::KP1-ART1^{OE}, B16^{CONTROL(Scr-6)} or B16^{ART1KO} cells were subcutaneously injected into the flank of C57BL/6 mice (1×10⁵ cells in 50 μl PBS) or immunodeficient nude mice. After tumors were palpable, tumor diameters were measured with digital calipers and the tumor volume determined by the formula (length×width²)/2). In the flank tumor model using B16-F10 CRISPR sublines, it was observed that some mice died prior to reaching maximum tumor volumes with evidence of metastatic dissemination. Where indicated, tumors were excised, weighed, and processed for immunofluorescence and/or flow cytometry analysis.

Antibody depletion of CD4 and CD8 T cells For CD8 and CD4 T cell depletion, a-CD8 (clone: 53-6.7, Bioxcell #BP0004-1) and a-CD4 (clone: GK1.5, Bioxcell #BP0003-1) antibodies were ip injected per mouse according to the following regimen: day-1 and day 3 (500 ug), then every 72 hrs till experiment endpoint (250 ug) (38). As a negative control to CD8 and CD4 depletion, mice from other groups received via ip injection InVivoPlus rat IgG2a isotype control, anti-trinitrophenol (clone 2A3, Bioxcell #BP0089) and InVivoPlus rat IgG2b isotype control, anti-keyhole limpet hemocyanin (clone LTF-2, Bioxcell #BP0090), respectively.

22C12 treatment of tumor bearing mice: For flank tumors, Intra-tumoral injections started when KP1-ART1^{OE} tumors became palpable on day 11 and every 72 hrs until day 23 after tumor inoculation. Mice were injected with 5 mg/kg ART1 antibody Clone 22C12 for group ‘22C12 Ab’ or Mouse IgG1 isotype control (BioXcell, Cat #BP0297) for group ‘Iso Ctrl Ab’. Tumor sizes were measured every 72 hrs and mice were sacrificed on day 25 after tumor inoculation when tumors were weighed and processed for flow cytometry staining. For the orthotopic lung tumor models, mice were injected i.v. with 0.5×10⁵ KP1-ART1^{OE} cells on day 0. Mice were intra-peritoneally (i.p.) injected with 25 mg/kg ART1 antibody Clone 22C12 for group ‘22C12 Ab’ or 25 mg/kg of humanized light chain 22C12 Ab 22C12 Ab (HuLC1) or Mouse IgG1 isotype control (BioXcell, Cat #BP0297) for group ‘Iso Ctrl Ab’. i.p. injections started from day 6 and continued every 72 hours till day 18 as indicated.

Cell Lines: The human cell lines H1650, A549, BEAS2B and HEK293 were obtained from ATCC and cultured in RPMI medium supplemented with 10% fetal bovine serum (FBS) and 1% penicillin streptomycin in a humidified 5% CO₂ incubator at 37° C. The mouse NSCLC cell line KP1 was previously generated from lung tumors of KRAS^{G12D/+}/p53^{-/-} (KP1) mice. Mouse LLC1 lung cancer cells and B16-F10 melanoma cells were obtained from ATCC and cultured in DMEM medium supplemented with 10% FBS and 1% penicillin streptomycin in a humidified 5% CO₂ incubator at 37° C.

Generation of KP1-ART1^{OE} and inducible hairpin stable cell lines: (i) Generation of KP1-ART1^{OE} cells: The pLVX-IRES-tdTomato vector is designed to constitutively coexpress the protein of interest and tdTomato from PCMV IE when transduced into mammalian cells. Before transduction, the vector was packaged into viral particles in HEK293T cells, using Lenti-XTM HT Packaging System (Catalog #632160 and 632161, Takara). The presence of tdTomato allows transductants to be visualized by fluorescence microscopy and sorted by flow cytometry. ART1 gene was overexpressed by using this construct (pLVX-IRES-td-tomato_ART1). Recombinant lentiviruses (LV) were generated from 293T cells (6×10⁶ cells/100 mm plate) by transient transfection of 7 ug of lentiviral short hairpin constructs (LT3GENIR) and the lentivirus packaging system (Clontech lenti-x single shot). LV particles were harvested 48 hours and 72 hours later, filtered through 0.45 μm filters, and concentrated by adding lenti-x concentrator (clontech). The LV were then incubated for 30 minutes at 4° C. and centrifuging at 1,500 g for 45 minutes at 4° C. LV particles were used to infect subconfluent cell cultures for 6 hours in the presence of 4 μg/mL polybrene (Sigma-Aldrich). Selection of viral infected cells expressing ART1 was done by sorting for tdtomato positive cells. (ii) Generation of shART1 cells: shRNA-LT3GENIR construct was used to knock down ART1 genes in KP1-ART 1^{OE} cells. The custom designed lentiviral construct expressed short hairpins targeting the Art1 gene and had GFP expression for selection. LV were generated as described in the previous section. Selection of viral infected cells expressing the shRNA was done by using 1 mg/mL G418 (Neomycin analogue, Sigma-Aldrich) in the media. To induce silencing, cells were treated cells with 1 ug/ml doxycycline which induced GFP and shRNA expression. ART1 shRNA construct #1, Art1_87 LT3GENIR. Antisense. Guide. Sequence TTTGATGTATTCACAGTTGTAT. (SEQ ID NO:17) 97mer.construct TGCTGTTGACAGTGAGCGATAGACATCTTTTCTCAAGAAATAGTGAAGCCACAGATGTATTTCTTGAGAAA AGATGTCTAGTGCCTACTGCCTCGGA (SEQ ID NO:18).

CRISPR-mediated gene knockout of ART1 in B16-F10 cells: CRISPR/Cas9 mediated knockout of ART1 in B16-F10 cells was performed using Sigma-Aldrich custom-made, ready-to-use DNA plasmids on the U6gRNA: CMV-CAS9-2A-tGFP backbone. Two plasmids containing gRNAs targeting regions in exon 3 of the ART1 gene were used to create the B16-F10 clones B16^{ART1KO (63-1)} (sequence 5′-3′: CCTGCGCTTTCGGCCAGCG; SEQ ID NO: 5) and B16^{ART1KO (42-1)} (sequence 5′-3′: CCAACAAAGTATACGCGGA; SEQ ID NO:19). A negative control plasmid was used to create the B16-F10 clone B16^{CONTROL (Scr-6)} (sequence 5′-3′: CGCGATAGCGCGAATATATT; SEQ ID NO:20). Briefly, B16-F10 cells were seeded in 12 well-plates and incubated for 48 hours to reach 80% confluency. Each CRISPR plasmid (0.5 μg DNA) were mixed with 3 □l TransIT-CRISPR reagent (Sigma-Aldrich) in 100 μl Opti-MEM medium (Gibco) and incubated at room temperature for 30 minutes. Mixture was added to the B16-F10 cells and incubated in a humidified 5% CO₂ incubator at 37° C. for 24 hours. Flow cytometry activated cell sorting (FACS) was used to sort transfected GFP-positive single cells into flat-bottom 96 well-plates. Clones were expanded and tested for ART1 surface expression by flow cytometry and immunofluorescence staining (FIG. 21).

Proliferation Assay: 1.4×10⁴ cells were plated in a 6-well plate. Cells were trypsinized and counted using Cellometer cell counting chambers (Nexcelom Bioscience) every day for four days. For experiments where cells were treated with NAD (20 μM, Sigma Aldrich, Catalog #N8285)+/−22C12 (20 μg/ml), FBS media was replenished with the mentioned reagents every 24 hours till endpoint.

Cell immunofluorescence: Adherent cells were plated in poly-D-lysine coated coverslips and were treated with serum free media for 12 hours before all experiments. Cells were washed with PBS-CM (1 mM MgCl₂, 0.1 mM CaCl₂)) and were fixed with 3.7% formaldehyde for 5 minutes (to prevent permeabilization) and incubated with blocking solution (5% BSA in 1×PBS) for 1 hour in a 37° C. air incubator. The cells were then treated with primary antibody ART1 (Purified ART1 antibody, Pocono, rabbit #2) (1:200) or Poly/Mono-ADP Ribose (CST, Clone: E6F6A Rabbit mAb Catalog #83732) (1:200) dissolved in 1% BSA (in 1×PBS, referred to as ‘cell IF antibody buffer’) for one hour in a 37° C. air incubator. Cells were washed with PBS-CM and then incubated with anti-rabbit fluorescent secondary antibody 1:500 (Thermofisher, #A10523) dissolved in ‘cell IF antibody buffer’ for 30 minutes. Cells were washed with PBS-CM and then stained with 1:1000 Hoechst (HOECHST3342, Thermofisher) in PBS-CM for 5 minutes. Post-washing with PBS-CM the samples fixed w/3.7% formaldehyde for 5 minutes. The samples were then washed in PBS and stored in PBS at 4° C. in the dark. Cell fluorescence microscopy was performed using a DMIRB inverted microscope (Leica Microsystems, Deerfield, IL), with a cooled charge-coupled device camera (Princeton Instruments, Trenton, NJ). Images were collected with a 40×1.25 numerical aperture objective. MetaMorph software (Universal Imaging, West Chester, PA) was used for image processing and quantification of MFI and background subtraction.

Cell Treatment with NAD⁺: Cells were treated with serum free media O/N before all experiments. Cells were then treated with NAD⁺ (Sigma Aldrich, Catalog #N8285) using dose dependent serial dilution (range of 0 to 50 uM) without or with dox (Art1 KD) or with Art1 blocking antibody (22C12).

Dissociation of mouse tissue samples: Mice were euthanized and tumor-bearing lungs were perfused by injection of 10 ml cold PBS through the right ventricle. Lungs and subcutaneous tumors were excised and chopped into small pieces using scalpels. Lung and tumor fragments were transferred to GentleMACS C tubes (Miltenyi, Catalog #130-096-334) after which lung dissociation mix (Miltenyi, Catalog #130-095-927) and mouse tumor dissociation mix (Miltenyi, Catalog #130-096-730) respectively was added to the tubes after preparation according to manufacturer's instructions. Lung and tumor fragments were enzymatically and mechanically digested using the gentleMACS Octo Dissociator with heaters (Miltenyi, Catalog #130-095-937) using program 37C_m_LDK_1 and 37C_m_TDK_1 respectively. Tissue homogenates were resuspended in RPMI 1640 (Corning, Catalog #15-040-CV) supplemented with 10% FBS and passed through a 70 UM strainer (Corning, Catalog #431751) to obtain a single cell suspension. Cells were pelleted and washed once in cold PBS. Cells were pelleted and resuspended in a working solution of RBC Lysis Buffer (eBioscience, Catalog #00-4300-54) and incubated for 2 minutes at room temperature. Cells were washed twice in PBS before proceeding with isolation of T cells or flow cytometry staining.

Dissociation of human tissue samples for flow cytometry: Fresh NSCLC patient tumor and adjacent normal tissues were obtained from the OR/Path on ice in DMEM+10% FBS+1% p/s. Tissue was washed twice with cold DMEM+10% FBS+1% p/s and chopped in DMEM supplemented with the following enzyme cocktail for tumor and normal, respectively (Collagenase I 50 U/mL, Collagenase II 20 U/mL, Collagenase IV 50 U/mL, Dnase I 50 Kunitz U/mL, Elastase 0.075 U/mL). Tissue was digested for 30 minutes at 37° C., filtered and centrifuged at 4° C. to collect pellet which was resuspended in Ack lysis buffer that was deactivated using RBC lysis with ice cold DMEM+10% FBS+1% p/s. Pellet was resuspended in FACS buffer to flow sorting staining.

ADP-ribosylation and NICD assay: Lung single cell suspensions were pelleted and resuspended in MACS buffer (AutoMACS Rinsing solution (Miltenyi, Catalog #130-091-222) supplemented with 0.5% BSA stock solution (Miltenyi, Catalog #130-091-376)). Isolation of T cells was performed by magnetic bead sorting using Pan T Cell Isolation Kit II, mouse (Miltenyi, Catalog #130-095-130) according to manufacturer's instructions. Cells were added to 48 well plates pre-coated with mouse ART1 (10 μg/ml) for 24 hours at 4° C. 48 hours prior to co-culture. 1×10⁶ T cells were resuspended in serum-free RPMI 1640 medium (Corning, Catalog #15-040-CV) containing 100 uM eNAD (Sigma Aldrich, Catalog #N2630), 5 μg/ml anti-ART2.2 antibody (s+16a, Biolegend, Catalog #149801), with or without 30 ug/ml anti-CD38 neutralizing antibody (Clone: NIMR-5, Novus Biologicals, Catalog #NBP2-59506) and with or without 20 ug/ml ART1 blocking 22C12 antibody added to wells. Cells were incubated at 37° C. for 2 hours. T cells were removed from plate by gentle pipetting and transferred to staining plates. T cells were stained with PE-conjugated anti-etheno-NAD antibody for 30 minutes at 4° C. (Clone: IG4, Santa Cruz, Catalog #sc-52666) followed by washing in FACS-buffer (PBS supplemented with 2 mM EDTA and 0.2% BSA) and staining with surface antibodies CD3 AF594 (Biolegend, Catalog #100240), CD8a BV605 (Biolegend, Catalog #100743), CD4 APC-Cy7 ((Biolegend, Catalog #100413), CD25 AF488 ((Biolegend, Catalog #102018), and P2X7R PE-Cy7 ((Biolegend, Catalog #148707) for 20 minutes at 4° C. DAPI (Biolegend, Catalog #422801) was added to the cells 10 minutes prior to acquisition on a FACSymphony Analyzer (BD Biosciences). Flow cytometry data was analyzed using the FlowJo software (FlowJo LLC, Becton Dickinson).

Flow Cytometry staining of human and mouse samples: Single cell suspensions derived from enzymatically digested tumor tissue and matched normal lung tissue from patients with lung adenocarcinoma were stained with fixable viability dye (eFluor 780) in PBS, 20 min, 4° C. Cells were resuspended in FcR blocking solution (Miltenyi, Catalog #130-092-575) for 5 minutes followed by addition of P2X7R primary antibody (Novus Biologicals, Clone: 7G1D6 Catalog #NBP2-61748) in FACS buffer for 20 min, 4° C. Cells were washed in FACS buffer and resuspended in Alexa Fluor® 488-conjugated Goat anti-mouse IgG secondary antibody (Biolegend, Catalog #405319) for 20 min at 4° C. Cells were washed in FACS buffer and incubated with a master mix of fluorophore-conjugated surface marker antibodies; CD3-Viogreen (Miltenyi, Catalog #130-113-704), CD8-PerCP-Vio700 (Miltenyi, Catalog #103-113-723), CD38-PE (eBioscience, Catalog #12-0389-42) for 20 min, 4° C.

Single cell suspensions from tumor-bearing mouse lungs or subcutaneous flank tumors were stained with fixable viability dye (eFluor 780) in PBS, 20 min, 4° C. For initial studies cells were washed in FACS buffer and resuspended in a master mix of fluorophore-conjugated surface marker antibodies: CD45-BD Horizon V500 (BD Biosciences, Catalog #561487), CD3-eFluor 450 (eBioscience, Catalog #48-0031-82), CD8b-PE-Vio770 (Miltenyi, Catalog #130-106-316), P2X7R-FITC (Miltenyi, Catalog #130-114-221). For further studies to characterize CD8 T cell proliferation and activation, master mixes of following conjugated antibodies: CD45-VioBlue (Miltenyi, Catalog #130-110-802), CD3-FITC (Miltenyi, Catalog #130-119-798), CD8b-PerCPVio700 (Miltenyi, Catalog #130-111-715), P2X7R-APC (Miltenyi, Catalog #130-114-330), CD279 (PD1)-PE (Miltenyi, Catalog #130-111-953). Cells were permeabilized for staining with Ki67 PE-Vio770 (Miltenyi, Catalog #130-120-419). To characterize memory T cell populations CD8 T cells the dispersed cells were surface stained in master mixes of following conjugated antibodies: CD69 PE (Miltenyi, Catalog #130-115-575), CD3-FITC (Miltenyi, Catalog #130-119-798), CD8b-PerCPVio700 (Miltenyi, Catalog #130-111-715), P2X7R-APC (Miltenyi, Catalog #130-114-330), CD62L-VioBlue (Miltenyi, Catalog #130-112-841) and CD44 PE-Vio770 (Miltenyi, Catalog #130-110-085). Memory CD8 T cell subsets were classified as follows; T_{CM}: CD62L+CD44+CD69−, T_{EM}: CD62L−CD44+CD69−, T_{RM}: CD62L-CD44+CD69+. Following surface staining, human and mouse cells were washed in FACS buffer and fixed using IC fixation buffer (Invitrogen) for 30 min, 4° C. To get absolute counts of immune populations, 30 ul counting beads (CountBright™ Absolute Counting Beads, 0.52×105 beads/50 ul, Invitrogen #C36950) were added before data acquisition per sample. Absolute counts were calculated using the formula; ((cell event count×counting bead volume)/(counting bead event count×cell volume))×counting bead concentration. Stained samples were acquired on a MACSQuant analyzer and flow cytometry data was analyzed using the FlowJo software (FlowJo LLC, Becton Dickinson).

Frozen Tissue Immunofluorescence: Mouse and biomarked patient samples were fixed in formaldehyde and kept 30% sucrose (in PBS) until the samples sink. Samples were embedded in OCT blocks and sectioned using cryostat (Leica). Sections were placed on charged slides, demarcated with barrier pen, and dehydrated in acetone. Sections were then blocked for 1 hr in blocking solution (0.25% Triton-x100+5% FBS in 1× PBS). The samples were subjected to overnight incubation in the dark at 4° C. with primary antibodies: Purified ART1 antibody (Purified ART1 antibody, Pocono, rabbit #2, 1:100), CD8 Polyclonal Antibody (#PA5-88265, 1:100), P2X7R (P2RX7 antibody cat #113544, biolegend, 1:100) dissolved in antibody buffer (5% FBS dissolved in 1×PBS).). Purified ART1 antibody (Purified ART1 antibody, Pocono, rabbit #2, 1:100) was used for Art1 staining of human patient samples. For CD8/P2X7R staining of human samples the following antibodies were used: CD8 Antibody (YTS105.18) (cat #NB200-578 Novus Biologicals, 1:100) and P2X7/P2RX7 Antibody (7G1D6) (NBP2-61748 Novus Biologicals, 1:100). Multiple sections of matched tumor and normal lungs were stained. The samples were washed multiple times in blocking solution and incubated in respective secondary antibodies (1:200 each secondary antibody) dissolved in antibody buffer for 1 hour in the dark. The samples were washed multiple times in blocking solution and incubated for 5 minutes with Hoechst (HOECHST3342, Thermofisher, 1:1000 in 1×PBS,). Sections were mounted using prolong gold mounting media (#P36934, Thermofisher). Sections were cured overnight at 20° C. in dark. Secondary only antibody stained sections were used to determine specificity of each primary antibody. Fluorescence microscopy was performed using Zeiss LSM 880 Laser Scanning Confocal Microscope. Multiple fields were acquired from multiple sections of each sample. ImageJ (NIH) was used for image processing, background subtraction, quantification MFI calculations, and cell counting.

Western blot Analyses: Cells were treated with serum free media O/N before all experiments. Cells were then treated with NAD⁺ (Sigma Aldrich, Catalog #N8285) using dose dependent serial dilution (range of 0 to 50 uM) without or with dox (Art1 KD) or with Art1 blocking antibody (22C12). Cells were washed with PBS and lysed in a mixture of 1× lysis buffer (cat #9803, CST) and Halt Protease & Phosphatase Inhibitor Single-Use Cocktail (cat #78442, Thermofisher). Cells were harvested by scraping, centrifuged to collect supernatant. For immunoblot analyses cellular proteins were resolved in 10% SDS/PAGE, transferred to nitrocellulose membranes, and probed with rabbit MAR/PAR antibody (CST #83732, 1:1000). Blots were acquired using MyECL Imager (Thermofisher). Pageruler plus prestained protein ladder, (10 to 250 kDa, #26619, Thermofisher) was used to determine weights of protein bands.

Quantitative RT-PCR analysis: Total RNA from cells was extracted with RNA Extraction_(QIAGEN RNeasy® Mini Kit). For initial studies with tumor cell lines, for each well 500 ng extracted RNA was reversely transcribed to cDNA using the RNA to cDNA EcoDry™ Premix (Random Hexamers) (catalogue #639546, Takara). Quantitative PCR was carried out using SYBR green master mix (iQ™ SYBR® Green Supermix, #1708884). The primer sequences for the human and mouse genes are listed in Table 5. C1000 Thermal Cycler (Bio-Rad) was used to perform real-time qPCR, and relative quantification performed using Bio-Rad CFX Manager software. For studies to compare P2X7R, P2X7R-a and P2X7R-k levels between lung derived T cells, KP1 lung derived CD8, KP1 lung derived CD4, and tumor lines (KP1, LLC1 and B16) TaqMan one step qPCR method was utilized. Primers were designed with the aid of Bioinformatics support from Thermofisher. 50 ng RNA was plated into each well and FAM-cojugated mouse primers for ‘gene of interest’ [P2X7R (Thermofisher Assay ID: Mm01199500_m1, targeting exon 2-3) or P2X7R-A (Thermofisher Assay ID: APXGWX4, targeting exon 1, custom made) or P2X7R-K (Thermofisher Assay ID: APAAF6U, targeting exon 1, custom made) and VIC conjugated GAPDH primers (Thermofisher Assay ID: Mm05724508_g1, targeting exon 4) were added to the reaction mix (iTaq Universal Probes One-Step Kit, Biorad #1725141). The relative mRNA expression levels were calculated using the 2^{-ΔΔct} method and normalized to relevant house-keeping gene (GAPDH).

RNA sequencing and gene expression analysis: CD8 T cells were isolated from untreated mice bearing KP1 lung tumors and RNA sequencing was performed as previously described (Markowitz et al., 2018). In order to display gene expression of select genes over the various treatment groups and cell types, FPKM for each treatment/cell type was imported into R (version 3.6.2). The function pheatmap was used to display gene expression as a heatmap and gene expression values were centered and scaled along rows by determining z-score for each value. Clustering was carried out using hierarchical clustering.

TCGA data analysis: cBioPortal was used to visualize and analyze transcriptomic data from the TCGA PanCancer Atlas (www.cbioportal.org). Gene expression and OS data of 503 lung adenocarcinoma samples were analyzed. Samples were stratified into mRNA expression data (Batch normalized from Illumina HiSeq_RNASeqV2) into ART1^{high} and ART1^{low} tumors using a z score threshold of ±1.0.

NAD-glo assay: NAD/NADH-Glo™ Assay (#G9071, Promega) kit was used. Histone (1.5 mg/mL), NAD (200 nM) (both from the kit) and recombinant ART1 (40 nM) or denatured ART1 (40 nM) enzymes (enzymes were cloned, expressed and purified by our collaborators at the Tri-Institutional Therapeutic Discovery Institute (TDI)) were added in a 96-well white opaque bottom plate. All the components were added to 1×PBS with a final reaction volume of 50 μL/well and incubated on a shaker at at 37° C. for 1 hour and equilibrated to room temperature for 5 minutes. The NAD/NADH-Glo Detection Reagent was prepared by mixing 1 mL reconstituted luciferin detection reagent, 5 uL reductase, 5 uL reductase substrate, 5 uL NAD cycling enzyme, and 5 uL NAD cycling substrate by gently inverting 5 times. 50 μL/well supernatant and 50 uL/well Detection Reagent were transferred to a new 96-well white luminometer plate, then incubated on a shaker in the dark at room temperature for 30 minutes. The luminescence of the samples was read on a luminometer.

22C12 antibody development and characterization: Antibody generation: Monoclonal ART1 binding and blocking antibodies were prepared utilizing the AlivaMab® Mouse (Ablexis, LLC) transgenic mouse platform comprising a human immunoglobulin repertoire. To generate functional human antibodies against ART1, purified recombinant human and mouse ART1 produced in HEK293 mammalian cells was used to immunize AlivaMab® Mouse, followed by the generation of hybridomas. Hybridoma supernatants from fused splenocytes expressing antibodies with the desired characteristics were identified utilizing a rigorous screening funnel developed by the TDI. First, hybridoma supernatants were screened by ELISA using plates coated with purified human ART1. Anti-human ART1 positive hybridoma supernatants were then tested for inhibition of purified human ART1 by a fluorescent NAD⁺ readout (Abcam, cat. ab176723). Anti-ART1 hybridoma supernatants were also tested for inhibition of human ART1 transiently expressed in HEK293 cells using the e-NAD-based ADP-ribosylation assay (Krebs et al., 2003). The hybridoma supernatants clone 22C12 was positive for inhibition of human ART1 in the biochemical and cell-based assays. Supernatants from 22C12 were then tested for binding to murine ART1 by ELISA and enzymatic inhibition of purified mouse ART1.

Binding affinity testing of 22C12 to human and mouse ART1: Following hybridoma subcloning and expansion of clone 22C12, the antibody was purified from hybridoma supernatant for potency ranking and affinity determination by bioluminescence (BLI). Range of concentrations of purified mouse and human light chain 22C12 antibodies and purified human or mouse ART1 was used to determine KD.

Testing for dose-dependent inhibition of surface ADP-ribosylation by 22C12: To determine potency in the cell-based functional assay, purified 22C12 antibody was incubated with HEK293 cells transiently transfected with human ART1 at different concentrations prior to treatment with e-NAD. Cell-surface ADP-ribosylation was then determined by flow cytometry and used to calculate IC₅₀ values.

### Summary

A majority of non-small-cell lung cancer (NSCLC) patients do not achieve durable clinical responses from immune checkpoint inhibitors suggesting the existence of additional resistance mechanisms. NAD-induced cell death (NICD) of P2X7-receptor (P2X7R)-expressing T cells mediated by mono-ADP-ribosyltransferases (ARTs) regulates immune homeostasis in inflamed tissues. An association was found between membranous ART1 tumor cell expression and reduced CD8 T cell infiltration, specifically a reduction in the P2X7R⁺CD8 T cell subset, in human lung adenocarcinomas. In a murine NSCLC model, genetic and pharmacologic antibody-mediated, ART1-inhibition slowed tumor growth in a CD8 T cell dependent manner and promotes tumor infiltration of activated P2X7R⁺CD8 T cells. In vitro, P2X7R⁺CD8 T cells were susceptible to ART1-mediated ADP-ribosylation and NICD, which was exacerbated upon blockade of the NAD⁺-degrading ADP-ribosyl cyclase CD38. ART1-mediated NICD provides for immune resistance in NSCLC and antibody-mediated targeting of ART1 can improve tumor control.

TABLE 2
Table 2. ART1 staining intensity in a NSCLC tissue microarray (TMA). Clinical
parameters and immune cell scoring of an adenocarcinoma tissue microarray.
Table compares tumors with strong ART1 staining vs tumors with weak or
moderate ART1 staining. Continuous variables are reported as median (interquartile
range) and categorical variables are reported as number (percent). Chi-
square test or Fisher's exact test (**) were used for pairwise comparison
of categorical variables. A Mann-Whitney U test was used for pairwise
comparisons of continuous variables.

| | ART1 staining intensity | | | | |
|---|---|---|---|---|---|
| | Strong | | Weak/moderate | | |
| | (n = 257) | | (n = 206) | | P value |
| Age (median, in years) | i9.7 | (63.7-75.6) | i8.6 | (60.1-75.6) | 0.157 |
| Gender (Female) | 167 | (65%) | 123 | (60%) | 0.244 |
| Smoking (current/former) | 205 | (81%) | 185 | (90%) | 0.004 |
| Pack/year (median) | 35 | (5.5-54.5) | 42.75 | (20-68) | 0.002 |

Pathologic Stage

| | | | | | |
|---|---|---|---|---|---|
| IA | 185 | (72%) | 128 | (62%) | 0.024 |
| IB | 72 | (28%) | 78 | (38%) | |

Pathologic features

| | | | | | |
|---|---|---|---|---|---|
| Necrosis | 37 | (14.4%) | 46 | (22.3%) | 0.027 |
| Lymphatic invasion | 62 | (24%) | 62 | (30%) | 0.149 |
| Vascular invasion | 68 | (26.5%) | 62 | (30%) | 0.387 |
| Visceral pleural invasion | 42 | (16.3%) | 48 | (23.3%) | 0.06 |

Predominant histologic subtype

| | | | | | |
|---|---|---|---|---|---|
| AIS/MIS | 6 | (2.3%) | 3 | (1.5%) | 0.737** |
| Lepidic | 19 | (7.4%) | 6 | (2.9%) | 0.034 |
| Acinar | 126 | (49%) | 79 | (38.3%) | 0.022 |
| Papillary | 67 | (26%) | 59 | (28.6%) | 0.537 |
| Micro-papillary | 6 | (2.3%) | 6 | (3.9%) | 0.333 |
| Solid | 23 | (8.9%) | 36 | (17.5%) | 0.006 |
| Mucinous | 9 | (3.5%) | 9 | (4.4%) | 0.632 |
| Colloid | 1 | (0.4%) | 6 | (2.9%) | 0.048** |

Mutational status

| Mutation EGFR or KRAS (n = 456) | 255 | | 201 | | |
|---|---|---|---|---|---|
| Wild type (EGFR− and KRAS−) | 137 | (53.7%) | 121 | (60.2%) | 0.026 |
| EGFR+ | 55 | (21.6%) | 24 | (11.9%) | |
| KRAS+ | 63 | (24.7%) | 56 | (27.9%) | |

Immune cell markers

| | | | | | |
|---|---|---|---|---|---|
| CD3 (>150 vs ≤150) | 22 | (8.5%) | 18 | (8.8%) | 0.921 |
| CD4 (>70 vs ≤70) | 30 | (11.7%) | 36 | (17.6%) | 0.072 |
| CD8 (>150 vs ≤150) | 28 | (11%) | 26 | (12.7%) | 0.55 |
| CD20 (>70 vs ≤70) | 23 | (9%) | 18 | (8.8%) | 0.908 |
| FoxP3 (>50 vs ≤50) | 22 | (8.6%) | 14 | (7%) | 0.5 |
| CD68 (>150 vs ≤150) | 54 | (21%) | 53 | (26%) | 0.179 |
| CD163 (>150 vs ≤150) | 21 | (8.2%) | 27 | (13.2%) | 0.083 |
| CD56 (Present vs absent) | 8 | (3%) | 6 | (3%) | 0.937 |

TABLE 3
Table 3. Localization of ART1 staining in a NSCLC tissue microarray (TMA). Clinical
parameters and immune cell scoring of an adenocarcinoma tissue microarray. Table
compares tumors with ART1 staining located to the cell surface or cell surface
and cytoplasm vs tumors with ART1 staining located to the cytoplasm only. Continuous
variables are reported as median (interquartile range) and categorical variables
are reported as number (percent). Chi-square test or Fisher's exact test (*)
were used for pairwise comparison of categorical variables. A Mann-Whitney U
test was used for pairwise comparisons of continuous variables.

| | ART1 staining localization | | | | |
|---|---|---|---|---|---|
| | Cell surface/ | | Cytoplasmic only | | |
| | Cytoplasmic (n = 48) | | (n = 445) | | P value |
| Age (median, in years) | 67 | (58-77) | 69 | (62-76) | |
| Gender (Female) | 24 | (56%) | 266 | (63%) | 0.33 |
| Smoking (current/former) | 38 | (88%) | 352 | (85%) | 0.51 |
| Pack/year (median) | 45 | (17-68) | 38 | (10-60) | 0.5 |

| | | |
|---|---|---|
| Resected with known Location | (n = 43) | (n = 420) |

Pathologic Stage

| | | | | | |
|---|---|---|---|---|---|
| IA | 28 | (65%) | 285 | (68%) | 0.71 |
| IB | 15 | (35%) | 135 | (32%) | |

Pathologic features

| | | | | | |
|---|---|---|---|---|---|
| Necrosis | 10 | (23%) | 73 | (17%) | 0.34 |
| Lymphatic invasion | 10 | (23%) | 114 | (27%) | 0.58 |
| Vascular invasion | 11 | (26%) | 119 | (28%) | 0.7 |
| Visceral pleural invasion | 5 | (12%) | 85 | (20%) | 0.17 |

Predominant histologic subtype

| | | | | | |
|---|---|---|---|---|---|
| AIS/MIS | 1 | (2%) | 8 | (2%) | 0.59* |
| Lepidic | 2 | (5%) | 23 | (5.5%) | 1* |
| Acinar | 19 | (44%) | 186 | (44.5%) | 1* |
| Papillary | 3 | (7%) | 123 | (29.5%) | 0.001* |
| Micro-papillary | 0 | | 14 | (3%) | 0.63 |
| Solid | 8 | (19%) | 51 | (12%) | 0.23 |
| Mucinous | 8 | (19%) | 10 | (2%) | <0.001 |
| Colloid | 2 | (5%) | 5 | (1%) | 0.13* |

Mutational status

| | | | | | |
|---|---|---|---|---|---|
| Wild type (EGFR− and KRAS−) | 27 | (67.5%) | 231 | (55.5%) | 0.091 |
| EGFR+ | 2 | (5%) | 77 | (18.5%) | |
| KRAS+ | 11 | (27.5%) | 108 | (26%) | |

Immune cell markers

| | | | | | |
|---|---|---|---|---|---|
| CD3 (>150 vs ≤150) | 4 | (%) | 36 | (9%) | 0.88 |
| CD4 (>70 vs ≤70) | 5 | (11.5%) | 61 | (14.5%) | 0.59 |
| CD8 (>50 vs ≤50) | 12 | (28%) | 190 | (56%) | 0.026 |
| CD20 (>70 vs ≤70) | 5 | (11.5%) | 36 | (9%) | 0.52 |
| FoxP3 (>50 vs ≤50) | 3 | (7%) | 33 | (8%) | 1* |
| CD68 (>150 vs ≤150) | 8 | (19%) | 99 | (24%) | 0.44 |
| CD163 (>150 vs ≤150) | 4 | (9.5%) | 44 | (10%) | 1* |
| CD56 (Present vs absent) | 1 | (2%) | 13 | (3%) | 0.77 |

TABLE 4
Immune cell scoring of NSCLC tissue microarray.

| Cell Marker | Infiltration score | Cell number cut-off |
|---|---|---|
| CD3 count in both | 1 (low) | <50 |
| tumor and stroma | 2 (intermediate) | 51-150 |
| | 3 (high) | >150 |
| CD4 count in both | 1 (low) | <20 |
| tumor and stroma | 2 (intermediate) | 21-70 |
| | 3 (high) | >70 |
| CD8 count in both | 1 (low) | <50 |
| tumor and stroma | 2 (intermediate) | 51-150 |
| | 3 (high) | >150 |
| CD20 count in both | 1 (low) | <20 |
| tumor and stroma | 2 (intermediate) | 21-70 |
| | 3 (high) | >70 |
| FoxP3 count in both | 1 (low) | <20 |
| tumor and stroma | 2 (intermediate) | 21-50 |
| | 3 (high) | >50 |
| CD68 count in both | 1 (low) | <50 |
| tumor and stroma | 2 (intermediate) | 51-100 |
| | 3 (high) | >100 |
| CD163 count in both | 1 (low) | <50 |
| tumor and stroma | 2 (intermediate) | 51-100 |
| | 3 (high) | >100 |
| CD56 count in both | 0 (absent) | absent |
| tumor and stroma | 1 (present) | present |

TABLE 5
Primers used for qPCR analysis

| | |
|---|---|
| Primer name | Primer sequence(5′→3′) |

Mouse primers for qPCR

| | |
|---|---|
| mART1 Primer | TTG ATG ACC AGT ATG CTG GCT |
| 2 REV | (SEQ ID NO: 8) |
| mART1 Primer | TTG TTG GCC TGA AAG TCT GAG |
| 2 FWD | (SEQ ID NO: 9) |
| mART1 Primer | CAG CGT CCT TTG ATG ACC AG |
| B4 REV | (SEQ ID NO: 10) |
| mART1 Primer | CCA CTG ATT GTT GGC CTG AG |
| B4 FWD | (SEQ ID NO: 11) |
| mGAPDH REV | AAT GTG TCC GTC GTG GAT CT |
| | (SEQ ID NO: 12) |
| mGAPDH FWD | GGT CCT CAG TGT AGC CCA AG |
| | (SEQ ID NO: 13) |
| GFP FWD | AAG CTG ACC CTG AAG TTC ATC TGC |
| | (SEQ ID NO: 14) |
| GFP REV | CTT GTA GTT GCC GTC GTC CTT GCC |
| | (SEQ ID NO: 15) |

Human primers for qPCR

| | |
|---|---|
| human ART1 REV | AGA AGA GGT CTC GTC GTG TGA |
| | SEQ ID NO: 85) |
| human ART1 FWD | GAT GCC TGC TAT GAT GTC TCT G |
| | (SEQ ID NO: 86) |
| human GAPDH REV | |
| human GAPDH FWD | |

### Example 4

### Results

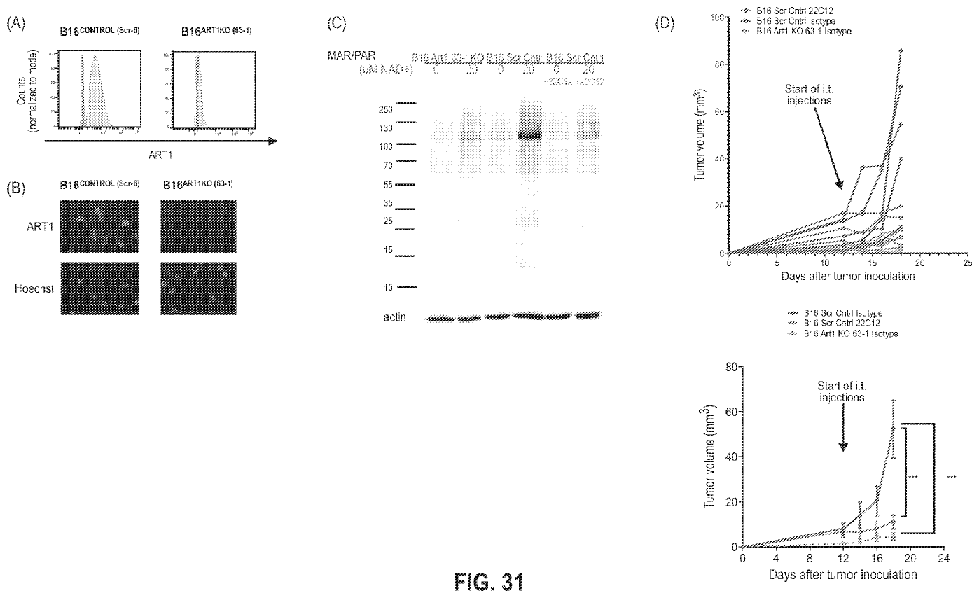

ART1 (Art1 KO) was knocked out using CRISPR/Cas9 in B16-F10 melanoma cells using a guide RNA (FIGS. 31A-B). Upon KO of Art1 in ‘63-1 KO’ B16 cells there was inhibition of cis-adp ribosylation when extracellular NAD+ was added to the cell medium. Similarly, cis-ADP ribosylation was also greatly inhibited in B16-F10 ‘Scr Control 6’ cells when co-treated with extracellular NAD+ and 22C12 antibody (FIG. 31C). Subcutaneous injection of ART1-expressing ‘Scr Control 6’ B16-F10 cells in syngeneic immune competent mice resulted in formation of rapidly growing flank tumors, while ART1-deficient B16-F10 ‘63-1 KO’ tumors and ‘Scr Control 6’ B16-F10 tumors intratumorally treated with 22C12 showed significant retardation in tumor growth (FIG. 31D).

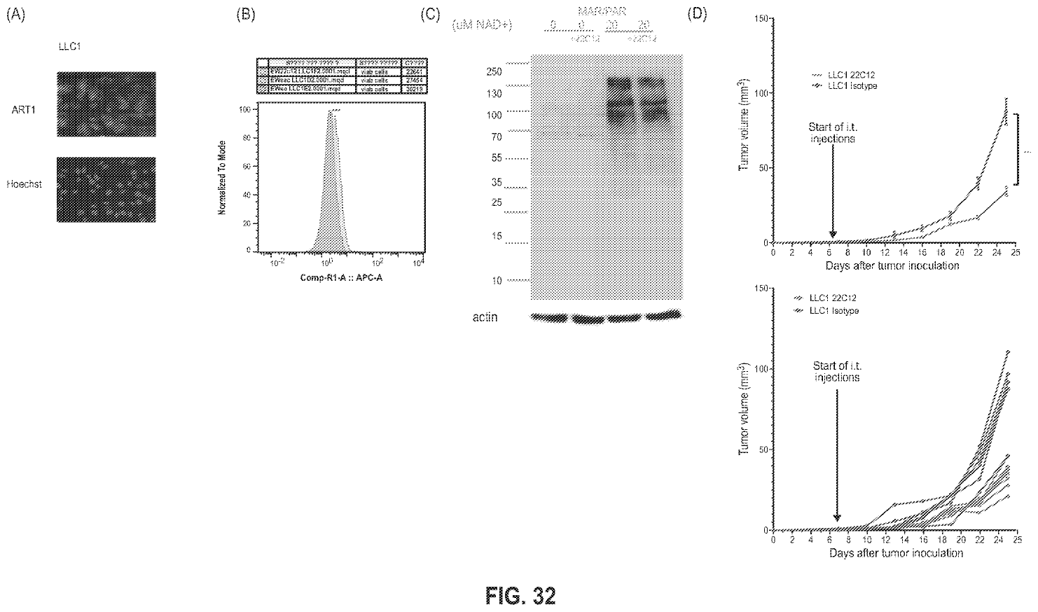

Surface ART1 expression was observed in a murine lung cancer line LLC1 (FIG. 32A). LLC1 cells bound to 22C12 in vitro consistent with the ART1 level on the surface of these cells (FIG. 32B). In vitro blockade of surface Art1 enzymatic activity by co-treating LLC1 cells with extracellular NAD⁺ and 22C12 partial blocked cis-ADP ribosylation signal (FIG. 32C).

Intratumoral injections of the 22C12 antibody (5 mg/kg), or the equivalent dose of isotype control antibody, into subcutaneously implanted LLC1 flank tumors starting on day 7 when tumors had become palpable, were performed. The injections were repeated every three days until day 25. It was observed that ART1 blockade resulted in a significantly reduction in tumor growth compared to tumors treated with isotype control antibody (FIG. 32D).

Methods Cell Lines

The B16 and LLC1 cell lines were obtained from ATCC and cultured in RPMI medium supplemented with 10% fetal bovine serum (FBS) and 1% penicillin streptomycin in a humidified 5% CO₂ incubator at 37° C. CRISPR-mediated gene knockout of ART1 in B16-F10 cells: CRISPR/Cas9 mediated knockout of ART1 in B16-F10 cells was performed using Sigma-Aldrich custom-made, ready-to-use DNA plasmids on the U6gRNA: CMV-CAS9-2A-tGFP backbone. Two plasmids containing gRNAs targeting regions in exon 3 of the ART1 gene were used to create the B16-F10 clones B16^{ART1KO (63-1)} (sequence 5′-3′: CCTGCGCTTTCGGCCAGCG; SEQ ID NO:5) and B16^{ART1KO (42-1)} (sequence 5′-3′: CCAACAAAGTATACGCGGA; SEQ ID NO:6). A negative control plasmid was used to create the B16-F10 clone B16^{CONTROL (Scr-6)} (sequence 5′-3′: CGCGATAGCGCGAATATATT; SEQ ID NO:7). Briefly, B16-F10 cells were seeded in 12 well-plates and incubated for 48 hours to reach 80% confluency. Each CRISPR plasmid (0.5 μg DNA) were mixed with 3 μl TransIT-CRISPR reagent (Sigma-Aldrich) in 100 μl Opti-MEM medium (Gibco) and incubated at room temperature for 30 min. Mixture was added to the B16-F10 cells and incubated in a humidified 5% CO₂ incubator at 37° C. for 24 hours. Flow cytometry activated cell sorting (FACS) was used to sort transfected GFP-positive single cells into flat-bottom 96 well-plates. Clones were expanded and tested for ART1 surface expression by flow cytometry and immunofluorescence staining (FIGS. 31A-B).

### Cell Immunofluorescence

Adherent cells were plated in poly-D-lysine coated coverslips and were treated with serum free media for 12 hours before all experiments. Cells were washed with PBS-CM (1 mM MgCl₂, 0.1 mM CaCl₂)) and were fixed with 3.7% formaldehyde for 5 minutes (to prevent permeabilization) and incubated with blocking solution (5% BSA in 1×PBS) for 1 hour in a 37° C. air incubator. The cells were then treated with primary antibody ART1 (Purified ART1 antibody, Pocono, rabbit #2) (1:200) dissolved in 1% BSA (in 1×PBS, referred to as ‘cell IF antibody buffer’) for one hour in a 37° C. air incubator. Cells were washed with PBS-CM and then incubated with anti-rabbit fluorescent secondary antibody 1:500 (Thermofisher, #A10523) dissolved in ‘cell IF antibody buffer’ for 30 minutes. Cells were washed with PBS-CM and then stained with 1:1000 Hoechst (HOECHST3342, Thermofisher) in PBS-CM for 5 min. Post-washing with PBS-CM the samples fixed w/3.7% formaldehyde for 5 minutes. The samples were then washed in PBS and stored in PBS at 4° C. in the dark.

Cell fluorescence microscopy was performed using a DMIRB inverted microscope (Leica Microsystems, Deerfield, IL), with a cooled charge-coupled device camera (Princeton Instruments, Trenton, NJ). Images were collected with a 40×1.25 numerical aperture objective. MetaMorph software (Universal Imaging, West Chester, PA) was used for image processing and quantification of MFI and background subtraction.

Cell Treatment with NAD⁺ and Western Blot

Cells were treated with serum free media O/N before all experiments. Cells were then treated with NAD⁺ (Sigma Aldrich, Catalog #N8285) using dose dependent serial dilution (0 and 20 uM) without or with Art1 blocking antibody (20 ug/ml) (22C12). Cells were washed with PBS and lysed in a mixture of 1× lysis buffer (cat #9803, CST) and Halt Protease & Phosphatase Inhibitor Single-Use Cocktail (cat #78442, Thermofisher). Cells were harvested by scraping, centrifuged to collect supernatant. For immunoblot analyses cellular proteins were resolved in 10% SDS/PAGE, transferred to nitrocellulose membranes, and probed with rabbit MAR/PAR antibody (CST #83732, 1:1000). Blots were acquired using MyECL Imager (Thermofisher). Pageruler plus prestained protein ladder (10 to 250 kDa, #26619, Thermofisher) was used to determine weights of protein bands.

Flow cytometry to detect binding of 22c12 Ab to LLC1 cells: Dispersed LLC1 cells were incubated in AF780 viability dye for 10 minutes (1:200 in PBS). Pellets were then washed and resuspended in FACS buffer. Cells were incubated with 22C12 (final concentration 20 μg/ml, in 4 degrees) for 30 minutes followed by anti-rabbit 568 (1:200, in 4 degrees) for 30 minutes. Samples were washed three times in FACS buffer, filtered and analyzed via flow cytometer.

### Animals

All animal work was done following a protocol approved by the Institutional Animal Care and Use Committee of New York Presbyterian Hospital/Weill Cornell Medical College (IACUC #2010-0050, 2015-0028). Wild type C57BL/6 mice (strain: C57BL/6NTac) were purchased from Taconic Biosciences. All mice were maintained under pathogen-free conditions in the Weill Cornell Medicine animal facility.

### Ectopic Flank Tumor Model

B16-F10 cells and LLC1 were subcutaneously injected into the flank of C57BL/6 mice (1×10⁵ cells in 0.05 mL PBS for B16 and 5×10⁵ cells in 0.05 mL for LLC1). After tumors were palpable, tumor diameters were measured with digital calipers and the tumor volume determined by the formula (length×width²)/2). 22C12 treatment of tumor bearing mice: For flank tumors, Intra-tumoral injections started when tumors became palpable on day 12 (for B16) and day 7 (for LLC1) and treated intratumorally every 72 hours from there onwards. Mice were injected with 5 mg/kg ART1 antibody Clone 22C12 for group ‘22C12 Ab’ or Mouse IgG2a isotype control (BioXcell, Cat #BE0085) for group ‘Iso Ctrl Ab’.

### Example 5

Humanization of the murine 22C12 antibody light-chain variable region was carried out followed by assessment of the antigen binding and functional properties of a panel of humanized candidate molecules.

To humanize the parental murine antibody 22C12 light-chain variable region, an approach was employed utilizing in silico sequence analysis and 3D structure generated by homology modeling to identify potentially suitable human light-chain frameworks. A panel of humanized antibodies were generated from candidate sequences designed to maximize the amount of human sequence in the humanized antibodies while retaining the parental antibody specificity and affinity. Selection of candidate humanized antibodies from the panel was determined based on evaluation of binding to the ART1 target antigen and functional inhibition of ART1 enzymatic activity.

The following are exemplary humanized framework regions for the light chain of antibody 22C12.

22C12 LC1
(SEQ ID NO: 48)
DIQLTQSPSFLSASVGDRVTITCRASSSVSYMHWYQQKPGTSPKR
LIYDTSKLASGVPSRFSGSGSGTEYTLTISSLQPEDFATYYCQQW
SSNPPTFGQGTKLEIKRTVAAPSVFIFPPSDEQLKSGTASVVCLL
NNFYPREAKVQWKVDNALQSGNSQESVTEQDSKDSTYSLSSTLTL
SKADYEKHKVYACEVTHQGLSSPVTKSFNRGEC
22C12 LC2
(SEQ ID NO: 49)
DIQMTQSPSSLSASVGDRVTITCSASSSVSYMHWYQQKPGTSPKR
LIYDTSKLASGVPSRFSGSGSGTDYTLTISSLQPEDFATYYCQQW
SSNPPTFGQGTKLEIKRTVAAPSVFIFPPSDEQLKSGTASVVCLL
NNFYPREAKVQWKVDNALQSGNSQESVTEQDSKDSTYSLSSTLTL
SKADYEKHKVYACEVTHQGLSSPVTKSFNRGEC
22C12 LC3
(SEQ ID NO: 50)
EIVLTQSPATLSLSPGERATLSCRASSSVSYMHWYQQKPGTSPRR
LIYDTSKLATGIPARFSGSGSGTDYTLTISSLEPEDFAVYYCQQW
SSNPPTFGQGTKLEIKRTVAAPSVFIFPPSDEQLKSGTASVVCLL
NNFYPREAKVQWKVDNALQSGNSQESVTEQDSKDSTYSLSSTLTL
SKADYEKHKVYACEVTHQGLSSPVTKSFNRGEC

### Example 6

### Properties of an Exemplary Anti-Human ART1 Antibody

TABLE 6
Molecular Pharmacology Properties

| | |
|---|---|
| PARAMETER | OBSERVATIONS |

Binding to Target

| | |
|---|---|
| Binding affinity to human ART1 | K_{D} = 1.36 nM |
| Cross-reactivity to cynomolgus ART1 | K_{D} = 1.46 nM |
| Cross-reactivity to mouse ART1 | K_{D} = 2.70 nM |
| Cell binding (HEK293 OE ART1) | EC₅₀ = 4.5 nM |

Molecular Selectivity

| | |
|---|---|
| Selectivity over ART1 paralogs | No binding to 1.3 μM |
| (ART3, ART4, ART5) | |

In vitro Functional Activity

| | |
|---|---|
| Inhibition of ART1 enzymatic activity | EC₅₀ = 4.5 nM |
| (cell based; HEK293 OE ART1) | |

Pharmacokinetics

| | |
|---|---|
| Clearance and serum half-life in mice | T_{1/2} (h) = 94.3; |
| (10 mg/kg, I.V.) | Cl (mL/day/kg) = 19.5 |

TABLE 7
In Vivo Efficacy

| | |
|---|---|
| PARAMETER | OBSERVATIONS |

Mouse Model

| | |
|---|---|
| Murine immune- | Significant reductions in lung weights, number |
| competent lung | of tumor nodules and tumor nodule size with |
| tumor models | increased T cell and DC infiltration |

TABLE 8
Biotherapeutic Attributes

| PARAMETER | OBSERVATIONS |
|---|---|
| Stable Pool Cell Line | 2.4 g/L titer from Chinese Hamster |
| Productivity | Ovary (CHO) |
| Cell Culture Product | >95% monomer in protein A (Pro-A) |
| Quality | eluate |
| Thermal Unfolding | Tₘ1-3 (° C.) = 67.4/74.4/86.0 |
| Charge Profile | Isoelectric point (pI) range 8.0-8.5 |
| Solubility/Appearance | Viscosity <20 centipoise (cP) |
| (150 mg/mL) | |

The full-length protein sequences of the TDI-Y-009 mAb are shown in Table 9. IMGT complementary determining region (CDR) definitions are indicated in Table 10.

TABLE 9
Protein Sequences of candidate
antibody TDI-Y-009.

| Chain Name | Full-length Protein Sequence |
|---|---|
| TDI-Y-009 Heavy | QVTLKESGPVLVKPTETLTLTCTVSGFSLS |
| Chain | NARMGVSWIRQPPGKALEWLAHIFSNDEKS |
| | YSTSLKSRLTISKDTSKSQVVLTMTNMDPV |
| | DTATYYCARIYGGDSWGYFDNWGQGTLVTV |
| | SSASTKGPSVFPLAPCSRSTSESTAALGCL |
| | VKDYFPEPVTVSWNSGALTSGVHTFPAVLQ |
| | SSGLYSLSSVVTVPSSSLGTKTYTCNVDHK |
| | PSNTKVDKRVESKYGPPCPPCPAPEFLGGP |
| | SVFLFPPKPKDTLMISRTPEVTCVVVDVSQ |
| | EDPEVQFNWYVDGVEVHNAKTKPREEQFNS |
| | TYRVVSVLTVLHQDWLNGKEYKCKVSNKGL |
| | PSSIEKTISKAKGQPREPQVYTLPPSQEEM |
| | TKNQVSLTCLVKGFYPSDIAVEWESNGQPE |
| | NNKTTPPVLDSDGSFFLYSRLTVDKSRWQY |
| | EGNVFSC SVMHEALHNHYTQKSLSLSLGK |
| | (SEQ ID NO: 64) |
| TDI-Y-009 Light | DIQLTQSPSFLSASVGDRVTITCRASSSVS |
| Chain | YMHWYQQKPGTSPKRLIYDTSKLASGVPSR |
| | FSGSGSGTEYTLTISSLQPEDFATYYCQQW |
| | SSNPPTFGQGTKLEIK RTVAAPSVFIFPP |
| | SDEQLKSGTASVVCLLNNFYPREAKVQWKV |
| | DNALQSGNSQESVTEQDSKDSTYSLSS TL |
| | TLSKADYEKHKVYACEVTHQGLSSPVTKSF |
| | NRGEC |
| | (SEQ ID NO: 65) |

Note:
Variable domains are indicated in bold font; CDRs are underlined; Constant domains are indicated innormal font.

TABLE 10
CDR sequences of TDI-Y-009.

| | LC | LC | LC | HC | HC | HC |
|---|---|---|---|---|---|---|
| Ab | CDR1 | CDR2 | CDR3 | CDR1 | CDR2 | CDR3 |
| hu22 | SSV | DTS | QQWS | GFSLS | IFSN | ARIYGGD |
| C12 | SY | | SNPPT | NARMG | DEK | SWGYFDN |
| | (SEQ | | (SEQ | (SEQ | (SEQ | (SEQ |
| | ID | | ID | ID | ID | ID |
| | NO: | | NO: | NO: | NO: | NO: |
| | 81) | | 30) | 66) | 67) | 68) |

### Characterization

In order to establish the target candidate profile (TCP), the antibody TDI-Y-009 was characterized for binding, pharmacokinetics, safety and efficacy. For in vivo efficacy and safety testing, the antibody was formatted with a mouse IgG1 Fc in order to lower the risk of eliciting anti-drug antibodies (ADA) as studies were run in immune competent mice for an extended time period.

### Binding to ART1: Human, Cynomolgus, Mouse

Sequence identity of cynomolgus and mouse ART1 to human ART1 are 95% and 76% respectively. Studies were run on Biacore 4000 using a C1 sensor chip. Briefly, goat anti-human antibody was amine coupled to the C1 sensor chip using standard NHS/EDC (0.1/0.4 M) activation, followed by the antibody at 50 μg/mL in 10 mM NaAc pH 5.0 and a 1 M ethanolamine blocking step. All studies were done in PBS (NaCl 137 mM, KCl 2.7 mM, Na2HPO4 10 mM, and KH2PO4 1.8 mM @ pH 7.4). Data were collected at 25° C. For each binding cycle, the mAb was diluted to 50 nM, 10 nM, 3.3 nM and 1.1 nM and captured for 60 seconds. The dissociation phase was monitored for 10 minutes followed by a regeneration step. A new aliquot of antibody was captured for each binding cycle. The processed sensorgram data were globally fit using a simple 1:1 interaction model. Similar high affinity binding of the mAb to human and cynomolgus ART1 was observed with comparable on and off rates. Binding to mouse ART1 was about 2-fold lower relative to human ART (Table 11).

TABLE 11
Binding kinetics of TDI-Y-009 to ART1.

| Antigen | kₒₙ (1/Ms) | k_{off} (1/s) | K_{D} |
|---|---|---|---|
| Human ART1 | 1.2 × 10⁵ | 1.6 × 10⁻⁴ | 1.36 nM |
| Cynomolgus ART1 | 1.8 × 10⁵ | 2.6 × 10⁻⁴ | 1.46 nM |
| Mouse ART1 | 1.2 × 10⁵ | 3.3 × 10⁻⁴ | 2.70 nM |

### Selectivity Over Human ART1 Paralogs

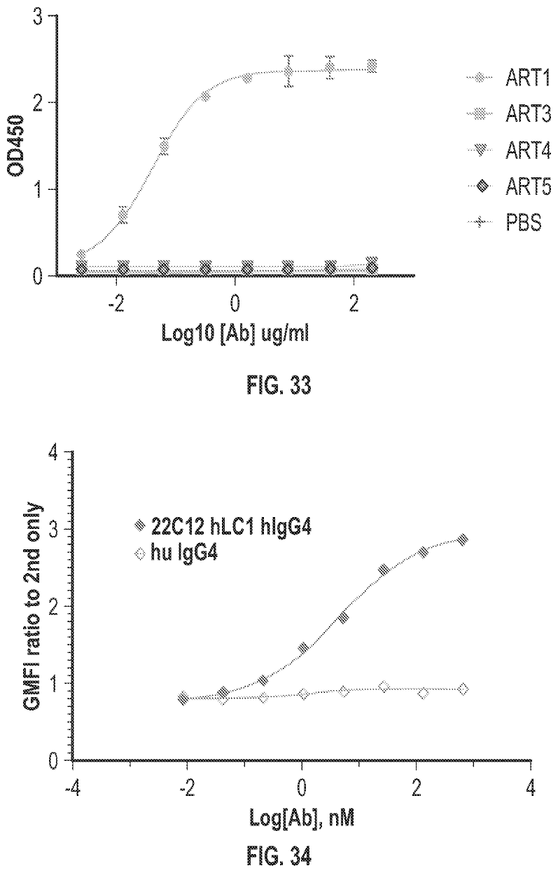

To assess selectivity of TDI-Y-009, binding to ART1 paralogs (ART3, ART4, ART5) was performed by ELISA. Briefly, ART1 or the paralogs were plated on a high binding ELISA plate at 2.5 μg/mL in 1×PBS and incubated o/n at 4° C. with shaking. After blocking, 1:5 serial dilutions of TDI-Y-009 mAb were added to the plate and incubated at RT for 90′. After washing, a 1:12,000 dilution of a goat anti-human secondary HRP labeled mAb was then added and incubated at RT for 1 hour before washing, adding the TMB substrate and reading out absorbance at 450 nM following color development and reaction quenching with acid. No binding to any of the ART1 paralogs was observed up to 1.3 uM (200 μg/mL) as indicated in FIG. 33.

### Binding to ART1 on Cells

Briefly, KP1-ART1^{OE} or KP1-ART1 knockdown cells were seeded on plates at 100,000 cells/well. Test antibodies starting at 100 μg/mL were serially diluted 1:5 then added to cells and incubated for 30 minutes on ice followed by washes. Cells were then stained with an anti-human IgG Fc and incubated for 30 minutes on ice followed by washes. Cells were resuspended in Flow buffer containing 1000× diluted SYTOX green dead stain and plates were read on Cytoflex flow cytometer. Dose-responsive binding to KP1-ART1^{OE} cells by TDI-Y-009 was observed (FIG. 34), but not to KP1-ART1 knockdown cells (data not shown).

### Epitope Mapping

An epitope mapping experiment using chemical crosslinking coupled with mass spectrometry (XL-MS) was performed. The results indicated that TDI-Y-009 cross-links with the following residues of huART1: S75, S77, T79, R80, R89, H92, and Y99. Corresponding paratope amino acids for TDI-Y-009 included: S51 (CDR2), T96 (CDR3) in the light chain; heavy chain CDR2 residues S55, K59, T63 and heavy chain CDR3 residues S105, and Y108. Based on modeling of huART1, the epitope is located in the N-terminal helical domain of huART1 and does not overlap with the ART1 active site or the NAD binding pocket located in the C-terminal beta-sheet domain of the enzyme. Engagement of the epitope residue Y99 (in helix 3 of the enzyme) may orient the antibody toward the NAD binding pocket possibly sterically hindering NAD binding. Alternatively, inhibition of ART1 activity may be due to conformational changes induced upon binding of Y-009, restricting substrate engagement. Although S75, S77, T79 and R80 are not conserved in mouse ART1, these residues form part of an insertion in human ART1 that is likely mobile and hence able to form chemical cross-links to a nearby bound antibody. Other epitope residues suggested by this method (R89, H92 and Y99) are conserved across mouse, cynomolgus and human ART1 and lie within a highly conserved linear region within the ART1 sequence.

### Mouse Pharmacokinetics

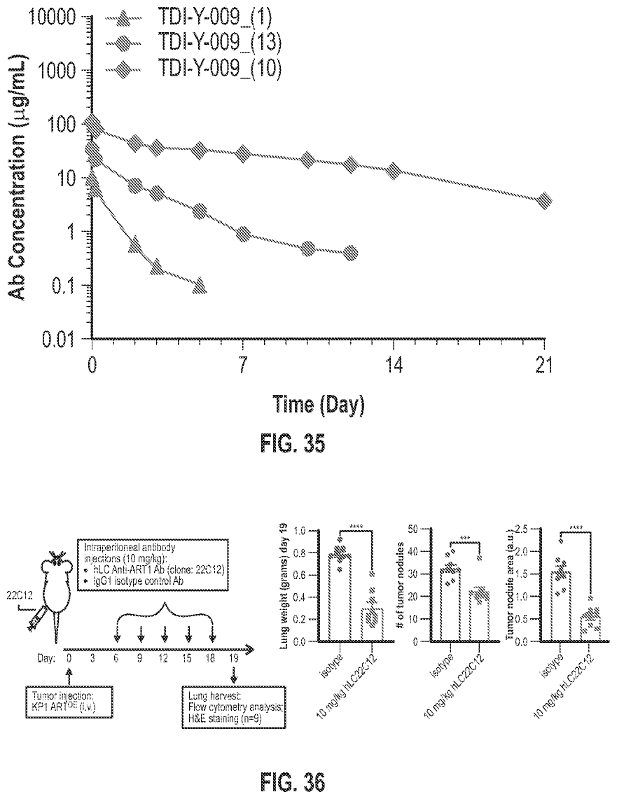

A study to assess the single-dose PK of TDI-Y-009 was performed in C57BL/6 mice. The plasma PK of the mAb was determined following a single IV bolus of 1, 3, or 10 mg/kg (n=5/group) using a nonvalidated ELISA assay. Briefly, goat anti-human IgG was coated on a plate, serum dilutions were then incubated followed by addition of secondary biotin labeled goat anti-human IgG-Fc and detection using streptavidin-HRP. Analysis was performed using WinNonlin software. As indicated in FIG. 35, TDI-Y-009 has nonlinear PK with dose-dependent clearance which is consistent with saturable target mediated clearance. The 10 mg/kg dose had a T_{1/2} (h)=94.3 which is typical for a well behaved antibody in mice exhibiting linear PK. This is suggestive of complete target saturation at this dose, though additional doses between 3 and 10 mg/kg would be needed to confirm this.

### In Vivo Efficacy Study

To confirm in vivo efficacy of the TDI-Y-009 mAb (formatted with a mIgG1 Fc), an orthotopic KP1-ART1^{OE} lung tumor model was run in which lung tumors are seeded by tail vein injection of tumor cells. The antibody was tested at 10 mg/kg to explore the effect at a lower dose than used in previous in vivo studies. Mice were administered TDI-Y-009 or an equivalent dose of isotype control antibody IP, starting on day 6 after tumor cell injection until day 18. Mice treated with the TDI-Y-009 antibody had significantly reduced lung tumor burden compared to the control mice, with reduced numbers of tumor nodules and reduction in tumor nodule area (FIG. 36). The use of mouse IgG1 Fc in reformatted TDI-Y-009 for this study ruled out a role for ADCC in eliciting anti-tumor activity.

### In Vivo Safety Study

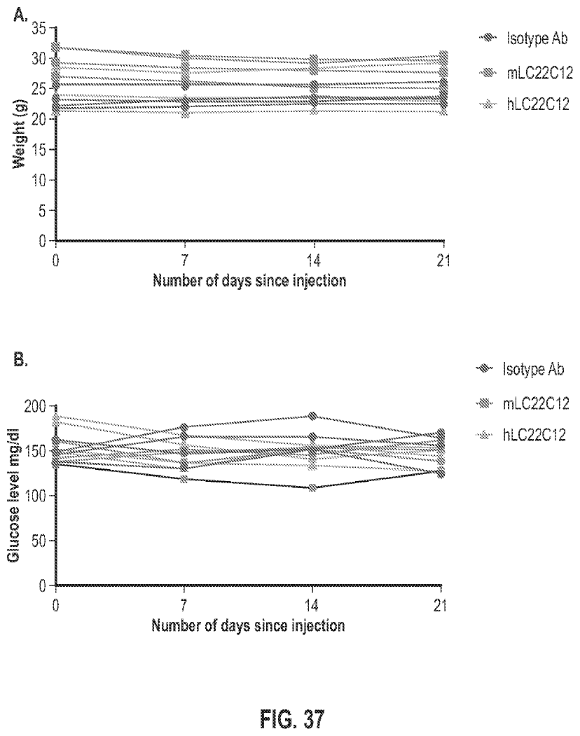

Briefly, tumor-naïve C57BL/6 mice were administered parental 22C12 (“mLC22C12”), TDI-Y-009 (“hLC22C12”) or isotype control antibody 25 mg/kg IP every three days for three weeks. Mice were closely monitored for weight loss with blood glucose levels measured at T=0 and then every week until the end of the study. Mice treated with the test antibodies were normal in appearance, activity, gait and alertness compared to mice treated with isotype control antibody (FIG. 37).

### Epitope Mapping

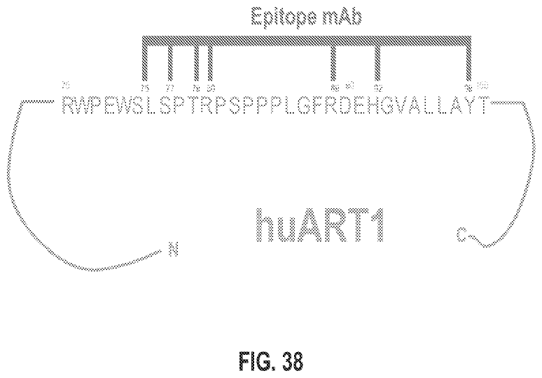

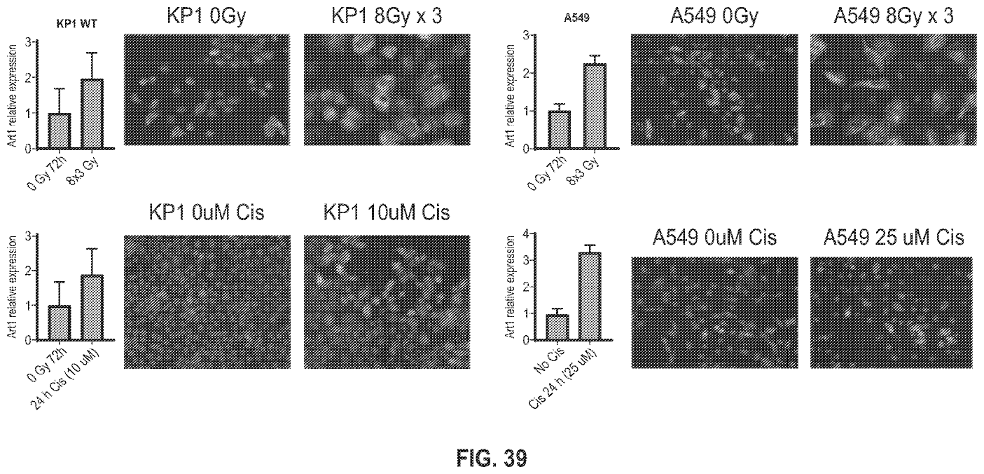

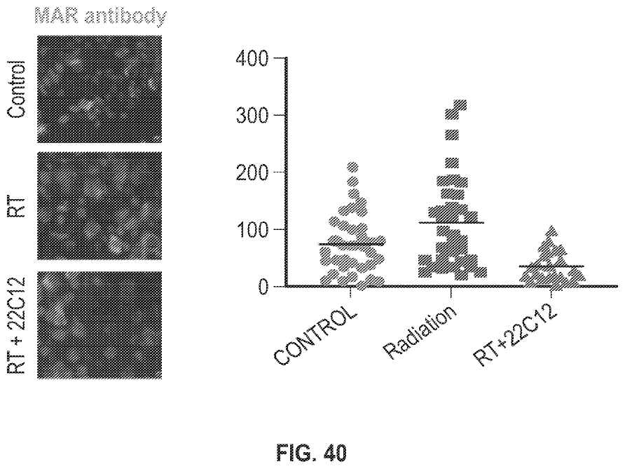

Using chemical cross-linking, High-Mass MALDI mass spectrometry and nLC-Orbitrap mass spectrometry the molecular interface between huART1 and 22C12-hLC was characterized. Briefly, each protein complex was incubated with deuterated cross-linkers and subjected to multi-enzymatic cleavage. After enrichment of the cross-linked peptides, the samples were analyzed by high-resolution mass spectrometry. The analysis indicates that the interaction includes the following amino acids on huART1 75, 77, 79, 80, 89, 92, 99. These results are illustrated in FIG. 38.

FIG. 39 depicts the huART1/mAb interaction. A PDB structure of huArt1 was generated using Swissmodel software and was colored in blue on the epitope site. Amino acids corresponding to 75-99 (SLSPTRPSPPPLGFRDEHGVALLAY; SEQ ID NO:70) of huART1 sequence provided.

In one embodiment, a framework region in the antibody or antigen binding fragment thereof, or polypeptide, comprises QVTLKESGPVLVKPTETLTLTCTVS (SEQ ID NO:71), VSWIRQP PGKALEWLAH (SEQ ID NO:72), SYSTSLKSRLTISKDTSKSQVVL™ TNMDPVDTATYYC (SEQ ID NO:73), or WGQGTLVTVSS (SEQ ID NO:74), a sequence having one, two, three, four, or five conservative amino acid substitutions, and optionally one two or three non-conservative substitutions.

In one embodiment, a framework region in the antibody or antigen binding fragment thereof, or polypeptide, comprises DIQLTQSPSFLSASVGDRVTITCRAS (SEQ ID NO:76), YMHWYQQKPGTS PKRLIY (SEQ ID NO:77), KLASGVPSRFSGSGSGTEYTLTISSLOPEDFATY YC (SEQ ID NO:78), FGQGTKLEIK (SEQ ID NO:79), or a sequence having one, two, three, four, or five conservative amino acid substitutions, and optionally one two or three non-conservative substitutions.

In one embodiment, an antibody or antigen binding fragment thereof, or polypeptide, comprises AS TKGPSVFPLAPCSRSTSESTAALGCLVKDYFPEPVTVSWNSGALTSGVHTFPAVLQSSGLYSLSSVVTVPSS SLGTKTYTCNVDHKPSNTKVDKRVESKYGPPCPPCPAPEFLGGPSVFLFPPKPKDTLMISRTPEVTCVVVDV SQEDPEVQFNWYVDGVEVHNAKTKPREEQFNSTYRVVSVLTVLHQDWLNGKEYKCKVSNKGLPSSIEKTISK AKGQPREPQVYTLPPSQEEMTKNQVSLTCLVKGFYPSDIAVEWE SNGQPENNKTTPPVLDSDGSFFLYSRLTVDKSRWQYEGNVFSC SVM HEALHNHYTQKSLSLSLGK (SEQ ID NO: 75), RTVAAPSVFIFPPSDEQLKSGT ASVVCLLNNFYPREAKVQWKVDNALQSGNSQESVTEQDSKDS TYSLSS TLTLSKADYEKHKVYACEVTHQGLSSPVTKSFNRGEC (SEQ ID NO:80), or a sequence having one, two, three, four, or five conservative amino acid substitutions, and optionally one two or three non-conservative substitutions, or a polypeptide with at least 80%, 82%, 84%, 85%, 87%, 88%, 89%, 90%, 92%, 94%, 95%, 96%, 97%, 98%, or 99% amino acid sequence identity thereto.

In one embodiment, an antibody or antigen binding fragment thereof, or polypeptide, comprises QVTLKESGPVLVKPTETLTLTCTVSGFSLSNARMGVSWIRQPPGKALEWLAH IFSNDEK SYSTSLKSRLTISKDTSKSQVVLTMTNMDPVDTATYYCARIYGGDSWGYFDNWGQGTLVTVSS (SEQ ID NO: 82), DIQLTQSPSFLSASVGDRVTITCRASSSVSYMHWYQQKPGTS PKRLIYDTS KLASGVPSRFSGSGSGTEYTLTISSLQPEDFATYYCQQWSSNPPTFGQGTKLEIK (SEQ ID NO:83) or a sequence having one, two, three, four, or five conservative amino acid substitutions, and optionally one two or three non-conservative substitutions, or a polypeptide with at least 80%, 82%, 84%, 85%, 87%, 88%, 89%, 90%, 92%, 94%, 95%, 96%, 97%, 98%, or 99% amino acid sequence identity thereto.

### REFERENCES

Adinolfi et al., Cancer Res., 75:635 (2015).
Adriouch et al., J. Immunol., 179:186 (2007).
Balducci et al., Am. J. Respir. Cell Mol. Biol., 21:337 (1999).
Boldrini et al., Oncol. Lett., 9:449 (2015).
Borges da Silva et al., Nature, 559:264 (2018).
Borges da Silva t al., Immunity, 53:158 (2020).
Burnstock et al., Int. Rev. Cytol., 240:31 (2004).
Cerami et al., Cancer Discov., 2:401 (2012).
Chen et al., Cancer Discov., 8:1156 (2018).
Chen et al., J. Thor. Onc., 11:S44 (2016).
Choi et al., Cell Rep., 10:1187 (2015).
Corda & Girolamo, EMBO J., 22:1953 (2003).
De Marchi et al., Oncogene, 38:3636 (2019).
Di Virgilio et al., Immunity, 47:15 (2017).
Di Virgilio et al., Nat. Rev. Cancer, 18:601 (2018).
Du et al., Infect. Immun., 69:3214 (2001).
Durrans et al., PLoS One, 10:e0129123 (2015).
Fabrizio et al., Cell Mol. Life Sci., 72:1209 (2015).
Gandhi et al., N. Engl. J. Med., 378:2078 (2018).
Gandini et al., Crit. Rev. Oncol. Hematol., 100:88 (2016).
Gao et al., Sci. Signal, 6: p11 (2013).
Ghiringhelli et al., Nat. Med., 15:1170 (2009).
Haag et al, J. Mol. Biol., 243:537 (1994).
Haag et al., Purinergic Signal, 3:71 (2007).
Haag et., Dev. Immunol., 9:197 (2002).
Horenstein t al., Oncoimmunology 2:e26246 (2013).
Hubert et al., J. Exp. Med., 207:2561 (2010).
Krebs et al., Anal. Biochem., 314:108 (2003).
Laing et al., Amino Acids, 41:257 (2011).
Lujambio, A., and Lowe, S. W. (2012). The microcosmos of cancer. Nature 482, 347-355.
Markowitz et al., JCI Insight, 3: (2018).
Mok et al., Lancet, 393:1819 (2019).
Nizard et al., Nat. Commun., 8:15221 (2017).
Okazaki et al., Biochemistry, 33:12828 (1994).
Paone et al., J. Biol. Chem., 281:17054 (2006).
Paone et al., Proc. Natl. Acad. Sci. USA, 99:8231 (2002).
Postow et al., N. Engl. J. Med., 366:925 (2012).
Reck et al., N. Engl. J. Med., 375;1823 (2016).
Sandoval-Montes & Santos-Argumedo, J. Leukoc. Biol., 77:513 (2005).
Scheuplein et al., J. Immunol., 182:2898 (2009).
Schwarz et al., PLos One, 7:e41269 (2012).
Seman et al., Immunity, 19:571 (2003).
Sharma & Allison, Science, 348:56 (2015).
Simon & Labarriere, Oncoimmunology, 7:e136482 (2017).
Song et al., Int. J. Oncol., 49:646 (2016).
Stark et al., Sci. Immunol., 3:(2018).
Stevens et al., Proc. Natl. Acad. Sci. USA, 106:19796 (2009).
Suzuki et al., J. Clin. Oncol., 31:490 (2013).
Tang et al., Int. J. Mol. Med., 32:130 (2013).
Taube et al., Mod. Pathol., 31:214 (2018).
Wang et al., J. Immunol., 153:4048 (1994).
Xu et al., Mol. Med. Rep., 15:1222 (2017).
Yang et al., Int. J. Mol. Med., 37:734 (2016).
Zamarin et al., Sci. Transl. Med., 6:226ra232 (2014).

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

1. An isolated cell comprising an expression cassette comprising a heterologous promoter operably linked to nucleic acid sequences encoding an anti-human ART1 antibody, or an antigen binding fragment thereof, or a polypeptide, that inhibits human ART1 activity, wherein the antibody, the antigen binding fragment thereof, or the polypeptide has:
i) a variable region comprising a first complementarity determining region (CDR) comprising GFSLSNARM (SEQ ID NO:66) operably linked to a second CDR comprising IFSNDEK (SEQ ID NO:67) operably linked to a third CDR comprising ARIYGGDSWGYFDN (SEQ ID NO:68); and/or
ii) a variable region comprising a first CDR comprising SSVSY (SEQ ID NO:81) operably linked to a second CDR comprising DTS (SEQ ID NO:29) operably linked to a third CDR comprising QQWSSNPPT (SEQ ID NO:30); or
iii) a variable region comprising a first CDR comprising GGSISSYY (SEQ ID NO:35) operably linked to a second CDR comprising ISTSGFT (SEQ ID NO:36) operably linked to a third CDR comprising ARDGWGRVFDI (SEQ ID NO:37); and/or
iv) a variable region comprising a first CDR comprising QSVSSSY (SEQ ID NO:42) operably linked to a second CDR comprising GAS (SEQ ID NO:43) operably linked to a third CDR comprising QQYGSST (SEQ ID NO:44).

2. The isolated cell of claim 1 wherein a heavy Ig chain comprising i) comprises a variable region comprising QVTLKESGPVLVKPTETLTLTCTVSGFSLSNARMGVSWIRQPPGKALEWLAHIFSNDEKS YSTSLKSRLTISKDTSKSQVVLTMTNMDPVDTATYYCARIYGGDSWGYFDNWGQGTLV TVSS (SEQ ID NO:1) or QVTLKESGPVLVKPTETLTLTCTVSGFSLSNARMGVSWIRQPPGKALEWLAH IFSNDEK SYSTSLKSRLTISKDTSKSQVVLTMTNMDPVDTATYYCARIYGGDSWGYFDNWGQGTL VTVSS (SEQ ID NO:82); or a light Ig chain ii) comprises a variable region comprising QIVLTQSPAIMSASLGEKVTMTCSASSSVSYMHWYQQKSGTSPKRWIYDTSKLASGVPA RFSGSGSGTSYSLTISSMEAGDAATYYCQQWSSNPPTFGAGTKLELK (SEQ ID NO:2) or DIQLTQSPSFLSASVGDRVTITCRASSSVSYMHWYQQKPGTS PKRLIYDTS KLASGVPSRFSGSGSGTEYTLTISSLQPEDFATYYCQQWSSNPPTFGQGTKLEIK (SEQ ID NO: 83).

3. The isolated cell of claim 1 wherein a heavy Ig chain comprising iii) comprises a variable region comprising: QVQLQESGPGLVKPSETLSLTCTVSGGSISSYYWSWIRQPAGKGLEWIGRISTSGFTNYN PSLKSRVTMSVDSSKNQFSLKLSSLTAADTAVYYCARDGWGRVFDIWGLGTMVTVSS (SEQ ID NO: 3); or a light Ig chain comprising iv) comprises a variable region comprising EIVLTQSPGTLSLSPGERATLSCRASQSVSSSYLAWYQQKPGQAPRLLIYGASSRATGIPD RFSGSGSGTDFTLTISRLEPEDFAVYYCQQYGSSTFGPGTKVDIK (SEQ ID NO:4).

4. The isolated cell of claim 1 wherein a light Ig chain comprising ii) comprises a variable region comprising
(SEQ ID NO: 69)
DIQLTQSPSFLSASVGDRVTITCRASSSVSYMHWYQQKPGTSPKR
LIYDTSKLASGVPSRFSGSGSGTEYTLTISSLQPEDFATYYCQQW
SSNPPTFGQGTKL;
(SEQ ID NO: 84)
DIQMTQSPSSLSASVGDRVTITCSASSSVSYMHWYQQKPGTSPKR
LIYDTSKLASGVPSRFSGSGSGTDYTLTISSLQPEDFATYYCQQW
SSNPPTFGQGTKL;
or
(SEQ ID NO: 87)
EIVLTQSPATLSLSPGERATLSCRASSSVSYMHWYQQKPGTSPRR
LIYDTSKLATGIPARFSGSGSGTDYTLTISSLEPEDFAVYYCQQW
SSNPPTFGQGTKL.

5.-7. (canceled)

8. A hybridoma comprising nucleic acid sequences encoding an anti-human ART1 monoclonal antibody that inhibits human ART1 activity, wherein the antibody has:
i) a variable region comprising a first complementarity determining region (CDR) comprising GFSLSNARM (SEQ ID NO:66) operably linked to a second CDR comprising IFSNDEK (SEQ ID NO:67) operably linked to a third CDR comprising ARIYGGDSWGYFDN (SEQ ID NO:68); and/or
ii) a variable region comprising a first CDR comprising SSVSY (SEQ ID NO:81) or SSSVSY (SEQ ID NO:28) operably linked to a second CDR comprising DTS (SEQ ID NO:29) operably linked to a third CDR comprising QQWSSNPPT (SEQ ID NO:30); or
iii) a variable region comprising a first CDR comprising GGSISSYY (SEQ ID NO:35) operably linked to a second CDR comprising ISTSGFT (SEQ ID NO:36) operably linked to a third CDR comprising ARDGWGRVFDI (SEQ ID NO:37); and
iv) a variable region comprising a first CDR comprising QSVSSSY (SEQ ID NO:42) operably linked to a second CDR comprising GAS (SEQ ID NO:43) operably linked to a third CDR comprising QQYGSST (SEQ ID NO:44).

9. An isolated nucleic acid comprising a promoter operably linked to a nucleotide sequence which encodes at least the variable region of a heavy or light Ig chain that binds human and/or mouse ART1, wherein the chain comprises:
i) a variable region comprising a first complementarity determining region (CDR) comprising GFSLSNARM (SEQ ID NO:66) operably linked to a second CDR comprising IFSNDEK (SEQ ID NO:67) operably linked to a third CDR comprising ARIYGGDSWGYFDN (SEQ ID NO:68); and/or
ii) a variable region comprising a first CDR comprising SSVSY (SEQ ID NO:81) operably linked to a second CDR comprising DTS (SEQ ID NO:29) operably linked to a third CDR comprising QQWSSNPPT (SEQ ID NO:30); or
iii) a variable region comprising a first CDR comprising GGSISSYY (SEQ ID NO:35) operably linked to a second CDR comprising ISTSGFT (SEQ ID NO:36) operably linked to a third CDR comprising ARDGWGRVFDI (SEQ ID NO:37); and/or
iv) a variable region comprising a first CDR comprising QSVSSSY (SEQ ID NO:42) operably linked to a second CDR comprising GAS (SEQ ID NO:43) operably linked to a third CDR comprising QQYGSST (SEQ ID NO:44).

10. An isolated antibody or antigen fragment thereof that binds human and mouse ART1, wherein the antibody or the antigen binding fragment thereof have:
i) a variable region comprising a first complementarity determining region (CDR) comprising GFSLSNARM (SEQ ID NO:66) operably linked to a second CDR comprising IFSNDEK (SEQ ID NO:67) operably linked to a third CDR comprising ARIYGGDSWGYFDN (SEQ ID NO:68); and/or
ii) a variable region comprising a first CDR comprising SSVSY (SEQ ID NO:81) operably linked to a second CDR comprising DTS (SEQ ID NO:29) operably linked to a third CDR comprising QQWSSNPPT (SEQ ID NO:30);
iii) a variable region comprising a first CDR comprising GGSISSYY (SEQ ID NO:35) operably linked to a second CDR comprising ISTSGFT (SEQ ID NO:36) operably linked to a third CDR comprising ARDGWGRVFDI (SEQ ID NO:37); and/or
iv) a variable region comprising a first CDR comprising QSVSSSY (SEQ ID NO:42) operably linked to a second CDR comprising GAS (SEQ ID NO:43) operably linked to a third CDR comprising QQYGSST (SEQ ID NO:44).

11. The antibody of claim 10 wherein the variable region of i) further comprises one or more framework regions comprising one or more of:
(SEQ ID NO: 24)
QVTLKESGPVLVKPTETLTLTCTVSGFSLS,
(SEQ ID NO: 25)
WIRQPPGKALEWLA,
(SEQ ID NO: 26)
RLTISKDTSKSQVVLTMTNMDPVDTATYYCAR,
(SEQ ID NO: 27)
WGQGTLVTVSS,
(SEQ ID NO: 71)
QVTLKESGPVLVKPTETLTLTCTVS,
(SEQ ID NO: 72)
VSWIRQPPGKALEWLAH,
(SEQ ID NO: 73)
SYSTSLKSRLTISKDTSKSQVVLTMTNMDPVDTATYYC,
or
(SEQ ID NO: 74)
WGQGTLVTVSS
[[,]].

12. The antibody of claim 10 wherein the variable region of ii) further comprises one or more framework regions comprising one or more of:
(SEQ ID NO: 31)
QIVLTQSPAIMSASLGEKVTMTCSA,
(SEQ ID NO: 32)
MHWYQQKSGTSPKRWIY,
(SEQ ID NO: 33)
KLASGVPARFSGSGSGTSYSLTISSMEAGDAATYYC,
(SEQ ID NO: 34)
FGAGTKLELK,
(SEQ ID NO: 76)
DIQLTQSPSFLSASVGDRVTITCRAS,
(SEQ ID NO: 77)
YMHWYQQKPGTSPKRLIY,
(SEQ ID NO: 78)
KLASGVPSRFSGSGSGTEYTLTISSLQPEDFATYYC,
or
(SEQ ID NO: 79)
FGQGTKLEIK.

13. The antibody of claim 10 wherein the variable region of ii) further comprises one or more framework regions comprising one or more of:
(SEQ ID NO: 51)
DIQLTQSPSFLSASVGDRVTITCRA,
(SEQ ID NO: 52)
MHWYQQKPGTSPKRLIY,
(SEQ ID NO: 53)
KLASGVPSRFSGSGSGTEYTLTISSLQPEDFATYYC,
or
(SEQ ID NO: 54)
FGQGTKLEIKRTVAAPSVFIFPPSDEQLKSGTASVVCLLNNFYPR
EAKVQWKVDNALQSGNSQESVTEQDSKDSTYSLSSTLTLSKADYE
KHKVYACEVTHQGLSSPVTKSFNRGEC

14. The antibody of claim 10 wherein the variable region of ii) further comprises one or more framework regions comprising one or more of:
(SEQ ID NO: 55)
DIQMTQSPSSLSASVGDRVTITCSA,
(SEQ ID NO: 56)
MHWYQQKPGTSPKRLIY,
(SEQ ID NO: 57)
KLASGVPSRFSGSGSGTDYTLTISSLQPEDFATYYC,
or
(SEQ ID NO: 58)
FGQGTKLEIKRTVAAPSVFIFPPSDEQLKSGTASVVCLLNNFYPR
EAKVQWKVDNALQSGNSQESVTEQDSKDSTYSLSSTLTLSKADYE
KHKVYACEVTHQGLSSPVTKSFNRGEC

15. The antibody of claim 10 wherein the variable region of ii) further comprises one or more framework regions comprising one or more of:
(SEQ ID NO: 59)
EIVLTQSPATLSLSPGERATLSCRA,
(SEQ ID NO: 60)
MHWYQQKPGTSPRRLIY,
(SEQ ID NO: 61)
KLATGIPARFSGSGSGTDYTLTISSLEPEDFAVYYC,
or
(SEQ ID NO: 62)
TFGQGTKLEIKRTVAAPSVFIFPPSDEQLKSGTASVVCLLNNFYP
REAKVQWKVDNALQSGNSQESVTEQDSKDSTYSLSSTLTLSKADY
EKHKVYACEVTHQGLSSPVTKSFNRGEC

16. The antibody of claim 10 wherein the variable region of iii) further comprises one or more framework regions comprising one or more of:
(SEQ ID NO: 38)
QVQLQESGPGLVKPSETLSLTCTVS,
(SEQ ID NO: 39)
WSWIRQPAGKGLEWIGR,
(SEQ ID NO: 40)
NYNPSLKSRVTMSVDSSKNQFSLKLSSLTAADTAVYYC,
or
(SEQ ID NO: 41)
WGLGTMVTVSS

17. The antibody of claim 10 wherein the variable region iv) further comprises one or more framework regions comprising one or more of:
(SEQ ID NO: 45)
EIVLTQSPGTLSLSPGERATLSCRAS,
(SEQ ID NO: 46)
LAWYQQKPGQAPRLLIY,
(SEQ ID NO: 47)
SRATGIPDRFSGSGSGTDFTLTISRLEPEDFAVYYC,
or
(SEQ ID NO: 63)
FGPGTKVDIK

18. A method to inhibit or treat cancer in a mammal, comprising: administering to a mammal a composition comprising an effective amount of an anti-human ART1 antibody or an antigen binding fragment thereof, or a polypeptide that binds human ART1.

19. The method of claim 18 wherein the cancer is lung cancer, colon cancer, melanoma, glioblastoma, breast cancer or colorectal cancer.

20. (canceled)

21. The method of claim 18, wherein the amount is effective to decrease tumor burden, inhibit metastases, increase survival, or any combination thereof.

22.-24. (canceled)

25. The method of claim 18, wherein the antibody, the antigen binding fragment thereof, or the polypeptide, has:
i) a variable region comprising a first complementarity determining region (CDR) comprising GFSLSNARM (SEQ ID NO:66) operably linked to a second CDR comprising IFSNDEK (SEQ ID NO:67) operably linked to a third CDR comprising ARIYGGDSWGYFDN (SEQ ID NO:68); and/or
ii) a variable region comprising a first CDR comprising SSVSY (SEQ ID NO:81) operably linked to a second CDR comprising DTS (SEQ ID NO:29) operably linked to a third CDR comprising QQWSSNPPT (SEQ ID NO:30); or
iii) a variable region comprising a first CDR comprising GGSISSYY (SEQ ID NO:35) operably linked to a second CDR comprising ISTSGFT (SEQ ID NO:36) operably linked to a third CDR comprising ARDGWGRVFDI (SEQ ID NO:37); and/or
iv) a variable region comprising a first CDR comprising QSVSSSY (SEQ ID NO:42) operably linked to a second CDR comprising GAS (SEQ ID NO:43) operably linked to a third CDR comprising QQYGSST (SEQ ID NO:44).

26.-35. (canceled)

36. The method of claim 30, wherein the antibody, the antigen binding fragment thereof, or the polypeptide has:
i) a variable region comprising a first complementarity determining region (CDR) comprising GFSLSNARM (SEQ ID NO:66) operably linked to a second CDR comprising IFSNDEK (SEQ ID NO:67) operably linked to a third CDR comprising ARIYGGDSWGYFDN (SEQ ID NO:68)); and/or
ii) a variable region comprising a first CDR comprising SSVSY (SEQ ID NO:81) or SSSVSY (SEQ ID NO:28) operably linked to a second CDR comprising DTS (SEQ ID NO:29) operably linked to a third CDR comprising QQWSSNPPT (SEQ ID NO:30); or
iii) a variable region comprising a first CDR comprising GGSISSYY (SEQ ID NO:35) operably linked to a second CDR comprising ISTSGFT (SEQ ID NO:36) operably linked to a third CDR comprising ARDGWGRVFDI (SEQ ID NO:37); and/or
iv) a variable region comprising a first CDR comprising QSVSSSY (SEQ ID NO:42) operably linked to a second CDR comprising GAS (SEQ ID NO:43) operably linked to a third CDR comprising QQYGSST (SEQ ID NO:44).

37.-43. (canceled)


SEQUENCE LISTING (110 sequences)

SEQ ID NO: 1 - protein, 122 aa, synthetic construct
QVTLKESGPVLVKPTETLTLTCTVSGFSLSNARMGVSWIRQPPGKALEWLAHIFSNDEKSYSTSLKSRLTISKDTSKSQVVLTMTNMDPVDTATYYCARIYGGDSWGYFDNWGQGTLVTVSS

SEQ ID NO: 2 - protein, 106 aa, synthetic construct
QIVLTQSPAIMSASLGEKVTMTCSASSSVSYMHWYQQKSGTSPKRWIYDTSKLASGVPARFSGSGSGTSYSLTISSMEAGDAATYYCQQWSSNPPTFGAGTKLELK

SEQ ID NO: 3 - protein, 117 aa, synthetic construct
QVQLQESGPGLVKPSETLSLTCTVSGGSISSYYWSWIRQPAGKGLEWIGRISTSGFTNYNPSLKSRVTMSVDSSKNQFSLKLSSLTAADTAVYYCARDGWGRVFDIWGLGTMVTVSS

SEQ ID NO: 4 - protein, 106 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSTFGPGTKVDIK

SEQ ID NO: 5 - DNA, 19 bp, synthetic construct
  source 1..19: mol_type = other DNA
cctgcgctttcggccagcg

SEQ ID NO: 6 - DNA, 19 bp, synthetic construct
  source 1..19: mol_type = other DNA
ccaacaaagtatacgcgga

SEQ ID NO: 7 - DNA, 20 bp, synthetic construct
  source 1..20: mol_type = other DNA
cgcgatagcgcgaatatatt

SEQ ID NO: 8 - DNA, 21 bp, synthetic construct
  source 1..21: mol_type = other DNA
ttgatgaccagtatgctggct

SEQ ID NO: 9 - DNA, 21 bp, synthetic construct
  source 1..21: mol_type = other DNA
ttgttggcctgaaagtctgag

SEQ ID NO: 10 - DNA, 20 bp, synthetic construct
  source 1..20: mol_type = other DNA
cagcgtcctttgatgaccag

SEQ ID NO: 11 - DNA, 20 bp, synthetic construct
  source 1..20: mol_type = other DNA
ccactgattgttggcctgag

SEQ ID NO: 12 - DNA, 20 bp, synthetic construct
  source 1..20: mol_type = other DNA
aatgtgtccgtcgtggatct

SEQ ID NO: 13 - DNA, 20 bp, synthetic construct
  source 1..20: mol_type = other DNA
ggtcctcagtgtagcccaag

SEQ ID NO: 14 - DNA, 24 bp, synthetic construct
  source 1..24: mol_type = other DNA
aagctgaccctgaagttcatctgc

SEQ ID NO: 15 - DNA, 24 bp, synthetic construct
  source 1..24: mol_type = other DNA
cttgtagttgccgtcgtccttgcc

SEQ ID NO: 16 - protein, 327 aa, synthetic construct
MQMPAMMSLLLVSVGLMEALQAQSHPITRRDLFSQEIQLDMALASFDDQYAGCAAAMTAALPDLNHTEFQANQVYADSWTLASSQWQERQARWPEWSLSPTRPSPPPLGFRDEHGVALLAYTANSPLHKEFNAAVREAGRSRAHYLHHFSFKTLHFLLTEALQLLGSGQRPPRCHQVFRGVHGLRFRPAGPRATVRLGGFASASLKHVAAQQFGEDTFFGIWTCLGAPIKGYSFFPGEEEVLIPPFETFQVINASRLAQGPARIYLRALGKHSTYNCEYIKDKKCKSGPCHLDNSAMGQSPLSAVWSLLLLLWFLVVRAFPDGPGLL

SEQ ID NO: 17 - DNA, 22 bp, synthetic construct
  source 1..22: mol_type = other DNA
tttgatgtattcacagttgtat

SEQ ID NO: 18 - DNA, 97 bp, synthetic construct
  source 1..97: mol_type = other DNA
tgctgttgacagtgagcgatagacatcttttctcaagaaatagtgaagccacagatgtatttcttgagaaaagatgtctagtgcctactgcctcgga

SEQ ID NO: 19 - DNA, 19 bp, synthetic construct
  source 1..19: mol_type = other DNA
ccaacaaagtatacgcgga

SEQ ID NO: 20 - DNA, 20 bp, synthetic construct
  source 1..20: mol_type = other DNA
cgcgatagcgcgaatatatt

SEQ ID NO: 21 - protein, 7 aa, synthetic construct
NARMGVS

SEQ ID NO: 22 - protein, 16 aa, synthetic construct
HIFSNDEKSYSTSLKS

SEQ ID NO: 23 - protein, 12 aa, synthetic construct
IYGGDSWGYFDN

SEQ ID NO: 24 - protein, 30 aa, synthetic construct
QVTLKESGPVLVKPTETLTLTCTVSGFSLS

SEQ ID NO: 25 - protein, 14 aa, synthetic construct
WIRQPPGKALEWLA

SEQ ID NO: 26 - protein, 32 aa, synthetic construct
RLTISKDTSKSQVVLTMTNMDPVDTATYYCAR

SEQ ID NO: 27 - protein, 11 aa, synthetic construct
WGQGTLVTVSS

SEQ ID NO: 28 - protein, 6 aa, synthetic construct
SSSVSY

SEQ ID NO: 29 - skipped

SEQ ID NO: 30 - protein, 9 aa, synthetic construct
QQWSSNPPT

SEQ ID NO: 31 - protein, 25 aa, synthetic construct
QIVLTQSPAIMSASLGEKVTMTCSA

SEQ ID NO: 32 - protein, 17 aa, synthetic construct
MHWYQQKSGTSPKRWIY

SEQ ID NO: 33 - protein, 36 aa, synthetic construct
KLASGVPARFSGSGSGTSYSLTISSMEAGDAATYYC

SEQ ID NO: 34 - protein, 10 aa, synthetic construct
FGAGTKLELK

SEQ ID NO: 35 - protein, 8 aa, synthetic construct
GGSISSYY

SEQ ID NO: 36 - protein, 7 aa, synthetic construct
ISTSGFT

SEQ ID NO: 37 - protein, 11 aa, synthetic construct
ARDGWGRVFDI

SEQ ID NO: 38 - protein, 25 aa, synthetic construct
QVQLQESGPGLVKPSETLSLTCTVS

SEQ ID NO: 39 - protein, 17 aa, synthetic construct
WSWIRQPAGKGLEWIGR

SEQ ID NO: 40 - protein, 38 aa, synthetic construct
NYNPSLKSRVTMSVDSSKNQFSLKLSSLTAADTAVYYC

SEQ ID NO: 41 - protein, 11 aa, synthetic construct
WGLGTMVTVSS

SEQ ID NO: 42 - protein, 7 aa, synthetic construct
QSVSSSY

SEQ ID NO: 43 - skipped

SEQ ID NO: 44 - protein, 7 aa, synthetic construct
QQYGSST

SEQ ID NO: 45 - protein, 26 aa, synthetic construct [sequence omitted]

SEQ ID NO: 46 - protein, 17 aa, synthetic construct
LAWYQQKPGQAPRLLIY

SEQ ID NO: 47 - protein, 36 aa, synthetic construct [sequence omitted]

SEQ ID NO: 48 - protein, 213 aa, synthetic construct
DIQLTQSPSFLSASVGDRVTITCRASSSVSYMHWYQQKPGTSPKRLIYDTSKLASGVPSRFSGSGSGTEYTLTISSLQPEDFATYYCQQWSSNPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 213 aa, synthetic construct
DIQMTQSPSSLSASVGDRVTITCSASSSVSYMHWYQQKPGTSPKRLIYDTSKLASGVPSRFSGSGSGTDYTLTISSLQPEDFATYYCQQWSSNPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 213 aa, synthetic construct
EIVLTQSPATLSLSPGERATLSCRASSSVSYMHWYQQKPGTSPRRLIYDTSKLATGIPARFSGSGSGTDYTLTISSLEPEDFAVYYCQQWSSNPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 25 aa, synthetic construct
DIQLTQSPSFLSASVGDRVTITCRA

SEQ ID NO: 52 - protein, 17 aa, synthetic construct
MHWYQQKPGTSPKRLIY

SEQ ID NO: 53 - protein, 36 aa, synthetic construct
KLASGVPSRFSGSGSGTEYTLTISSLQPEDFATYYC

SEQ ID NO: 54 - protein, 117 aa, synthetic construct
FGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 25 aa, synthetic construct
DIQMTQSPSSLSASVGDRVTITCSA

SEQ ID NO: 56 - protein, 17 aa, synthetic construct
MHWYQQKPGTSPKRLIY

SEQ ID NO: 57 - protein, 36 aa, synthetic construct
KLASGVPSRFSGSGSGTDYTLTISSLQPEDFATYYC

SEQ ID NO: 58 - protein, 117 aa, synthetic construct; identical to SEQ ID NO: 54

SEQ ID NO: 59 - protein, 25 aa, synthetic construct
EIVLTQSPATLSLSPGERATLSCRA

SEQ ID NO: 60 - protein, 17 aa, synthetic construct
MHWYQQKPGTSPRRLIY

SEQ ID NO: 61 - protein, 36 aa, synthetic construct
KLATGIPARFSGSGSGTDYTLTISSLEPEDFAVYYC

SEQ ID NO: 62 - protein, 118 aa, synthetic construct
TFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 10 aa, synthetic construct
FGPGTKVDIK

SEQ ID NO: 64 - protein, 448 aa, synthetic construct
QVTLKESGPVLVKPTETLTLTCTVSGFSLSNARMGVSWIRQPPGKALEWLAHIFSNDEKSYSTSLKSRLTISKDTSKSQVVLTMTNMDPVDTATYYCARIYGGDSWGYFDNWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNKTTPPVLDSDGSFFLYSRLTVDKSRWQYEGNVFSCSVMHEALHNHYTQKSLSLSLGK

SEQ ID NO: 65 - protein, 213 aa, synthetic construct; identical to SEQ ID NO: 48

SEQ ID NO: 66 - protein, 10 aa, synthetic construct
GFSLSNARMG

SEQ ID NO: 67 - protein, 7 aa, synthetic construct
IFSNDEK

SEQ ID NO: 68 - protein, 14 aa, synthetic construct
ARIYGGDSWGYFDN

SEQ ID NO: 69 - protein, 103 aa, synthetic construct
DIQLTQSPSFLSASVGDRVTITCRASSSVSYMHWYQQKPGTSPKRLIYDTSKLASGVPSRFSGSGSGTEYTLTISSLQPEDFATYYCQQWSSNPPTFGQGTKL

SEQ ID NO: 70 - protein, 25 aa, synthetic construct
SLSPTRPSPPPLGFRDEHGVALLAY

SEQ ID NO: 71 - protein, 25 aa, synthetic construct
QVTLKESGPVLVKPTETLTLTCTVS

SEQ ID NO: 72 - protein, 17 aa, synthetic construct
VSWIRQPPGKALEWLAH

SEQ ID NO: 73 - protein, 38 aa, synthetic construct
SYSTSLKSRLTISKDTSKSQVVLTMTNMDPVDTATYYC

SEQ ID NO: 74 - protein, 11 aa, synthetic construct
WGQGTLVTVSS

SEQ ID NO: 75 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNKTTPPVLDSDGSFFLYSRLTVDKSRWQYEGNVFSCSVMHEALHNHYTQKSLSLSLGK

SEQ ID NO: 76 - protein, 26 aa, synthetic construct
DIQLTQSPSFLSASVGDRVTITCRAS

SEQ ID NO: 77 - protein, 18 aa, synthetic construct
YMHWYQQKPGTSPKRLIY

SEQ ID NO: 78 - protein, 36 aa, synthetic construct; identical to SEQ ID NO: 53

SEQ ID NO: 79 - protein, 10 aa, synthetic construct
FGQGTKLEIK

SEQ ID NO: 80 - protein, 107 aa, synthetic construct [sequence omitted]

SEQ ID NO: 81 - protein, 5 aa, synthetic construct
SSVSY

SEQ ID NO: 82 - protein, 122 aa, synthetic construct; identical to SEQ ID NO: 1

SEQ ID NO: 83 - protein, 106 aa, synthetic construct
DIQLTQSPSFLSASVGDRVTITCRASSSVSYMHWYQQKPGTSPKRLIYDTSKLASGVPSRFSGSGSGTEYTLTISSLQPEDFATYYCQQWSSNPPTFGQGTKLEIK

SEQ ID NO: 84 - protein, 103 aa, synthetic construct
DIQMTQSPSSLSASVGDRVTITCSASSSVSYMHWYQQKPGTSPKRLIYDTSKLASGVPSRFSGSGSGTDYTLTISSLQPEDFATYYCQQWSSNPPTFGQGTKL

SEQ ID NO: 85 - DNA, 21 bp, synthetic construct
  source 1..21: mol_type = other DNA
agaagaggtctcgtcgtgtga

SEQ ID NO: 86 - DNA, 22 bp, synthetic construct
  source 1..22: mol_type = other DNA
gatgcctgctatgatgtctctg

SEQ ID NO: 87 - protein, 103 aa, synthetic construct
EIVLTQSPATLSLSPGERATLSCRASSSVSYMHWYQQKPGTSPRRLIYDTSKLATGIPARFSGSGSGTDYTLTISSLEPEDFAVYYCQQWSSNPPTFGQGTKL

SEQ ID NO: 88 - protein, 325 aa, synthetic construct
MKIPAMMSLLLVSVGLRDGVQVQSYSISQLDIFSQETPLDMAPASFDDQYAGCLADMTAALPDLNHSEFQANKVYADGWAQANNQWQERRAWGSVWGSLPPSPPGFRDEHGVALLAYTANSPLHKEFNAAVREAGRSRAHYLHHFSFKTLHFLLTEALQLLRSHRSRGCQQVYRGVHGLRFRPAGPGATVRLGGFASASLKNVAAQQFGEDTFFGIWTCLGAPIRGYSFFPEEEEVLIPPFETFQVINTSRPTQGPARIYLRALGKRSTYNCEYIKEKKCRSGPCWLGSSAPGSISASCSLLLLLLFLVLSALPENPGLQQLTRC

SEQ ID NO: 89 - skipped

SEQ ID NO: 90 - protein, 298 aa, synthetic construct
MQMPAMMSLLLVSVGLMEALQAQSHPITRRDLFSQEIQLDMALASFDDQYAGCAAAMTAALPDLNHTEFQANQVYADSWTLASSQWQERQARWPEWSLSPTRPSPPPLGFRDEHGVALLAYTANSPLHKEFNAAVREAGRSRAHYLHHFSFKTLHFLLTEALQLLGSGQRPPRCHQVFRGVHGLRFRPAGPRATVRLGGFASASLKHVAAQQFGEDTFFGIWTCLGAPIKGYSFFPGEEEVLIPPFETFQVINASRLAQGPARIYLRALGKHSTYNCEYIKDKKCKSGPCHLDNSAMG

SEQ ID NO: 91 - protein, 239 aa, synthetic construct
MPAMMSLLLVSVGLQQSIDFSQELDMAASFDDQYAGCAMTAALPDLNHEFQANVYADWAQWQERAWPPSPPGFRDEHGVALLAYTANSPLHKEFNAAVREAGRSRAHYLHHFSFKTLHFLLTEALQLLSRCQVRGVHGLRFRPAGPATVRLGGFASASLKVAAQQFGEDTFFGIWTCLGAPIGYSFFPEEEVLIPPFETFQVINSRQGPARIYLRALGKSTYNCEYIKKKCSGPCLSAG

SEQ ID NO: 92 - protein, 293 aa, synthetic construct
MKIPAMMSLLLVSVGLRDGVQVQSYSISQLDIFSQETPLDMAPASFDDQYAGCLADMTAALPDLNHSEFQANKVYADGWAQANNQWQERRAWGSVWGSLPPSPPGFRDEHGVALLAYTANSPLHKEFNAAVREAGRSRAHYLHHFSFKTLHFLLTEALQLLRSHRSRGCQQVYRGVHGLRFRPAGPGATVRLGGFASASLKNVAAQQFGEDTFFGIWTCLGAPIRGYSFFPEEEEVLIPPFETFQVINTSRPTQGPARIYLRALGKRSTYNCEYIKEKKCRSGPCWLGSSAPG

SEQ ID NO: 93 - protein, 7 aa, synthetic construct
NAHMGVS

SEQ ID NO: 94 - protein, 7 aa, synthetic construct
QARMGIS

SEQ ID NO: 95 - protein, 7 aa, synthetic construct
NGRMGVS

SEQ ID NO: 96 - protein, 16 aa, synthetic construct
HIFSNDEKSYSTSIKS

SEQ ID NO: 97 - protein, 16 aa, synthetic construct
HLFSNDEKSYSTSIKS

SEQ ID NO: 98 - protein, 16 aa, synthetic construct
HIFTNDEKSYSSSLKS

SEQ ID NO: 99 - protein, 13 aa, synthetic construct
IYGGADSWGYFEN

SEQ ID NO: 100 - protein, 12 aa, synthetic construct
IYGGDSWAYFDN

SEQ ID NO: 101 - protein, 12 aa, synthetic construct
LYGIDSWGYFDN

SEQ ID NO: 102 - protein, 10 aa, synthetic construct
GFSISNARMG

SEQ ID NO: 103 - protein, 10 aa, synthetic construct
GFSASNTRMG

SEQ ID NO: 104 - protein, 10 aa, synthetic construct
GFSISNLRMA

SEQ ID NO: 105 - protein, 7 aa, synthetic construct
LFSNDEK

SEQ ID NO: 106 - protein, 7 aa, synthetic construct
IFSNEDK

SEQ ID NO: 107 - protein, 14 aa, synthetic construct
GRIYGGDSWGYFDN

SEQ ID NO: 108 - protein, 14 aa, synthetic construct
ARIYAADSWGYFDN

SEQ ID NO: 109 - protein, 14 aa, synthetic construct
IRAYGGDSWLYFDN

SEQ ID NO: 110 - protein, 31 aa, synthetic construct
RWPEWSLSPTRPSPPPLGFRDEHGVALLAYT